SEQUENCE LISTING (21 sequences)

SEQ ID NO: 1 - RNA, 24 nt, Homo sapiens
  misc_feature 1..24: note = Description of sequence: human hsa-miR-155-5p MIMAT0000646
  source 1..24: mol_type = other RNA
ttaatgctaatcgtgataggggtt

SEQ ID NO: 2 - RNA, 24 nt, Mus musculus
  misc_feature 1..24: note = Description of sequence: mouse miR-155-5p MIMAT0000646
  source 1..24: mol_type = other RNA
ttaatgctaattgtgataggggtt

SEQ ID NO: 3 - RNA, 22 nt, Homo sapiens
  misc_feature 1..22: note = Description of sequence: human hsa-miR-155-3p MIMAT0004658
  source 1..22: mol_type = other RNA
ctcctacatattagcattaaca

SEQ ID NO: 4 - RNA, 22 nt, Mus musculus
  misc_feature 1..22: note = Description of sequence: mouse -miR-155-3p MIMAT0004658
  source 1..22: mol_type = other RNA
ctcctacctgttagcattaaca

SEQ ID NO: 5 - RNA, 65 nt, synthetic construct
  misc_feature 1..65: note = Description of sequence: hsa-miR-155 stem loop MI0000681
  source 1..65: mol_type = other RNA
ctgttaatgctaatcgtgataggggtttttgcctccaactgactcctacatattagcattaacag

SEQ ID NO: 6 - RNA, 24 nt, synthetic construct
  misc_feature 1..24: note = Description of sequence: modified hsa-miR-155-5p
  source 1..24: mol_type = other RNA
ggaatgctaatcgtgataggggtt

SEQ ID NO: 7 - RNA, 22 nt, synthetic construct
  misc_feature 1..22: note = Description of sequence: modified hsa-miR-155-3p
  source 1..22: mol_type = other RNA
ttcctacatattagcattaaca

SEQ ID NO: 8 - RNA, 65 nt, synthetic construct
  misc_feature 1..65: note = Description of sequence: modified hsa-miR-155 stem loop
  source 1..65: mol_type = other RNA
ctgttaatgctaatcgtgatagggatttttgcctccaactgattcctacatattagcattaacag

SEQ ID NO: 9 - RNA, 20 nt, synthetic construct
  misc_feature 1..20: note = Description of sequence: miR-155-3p isomiR
  source 1..20: mol_type = other RNA
acctacgattatacatcctc

SEQ ID NO: 10 - RNA, 20 nt, synthetic construct
  misc_feature 1..20: note = Description of sequence: miR-155-3p isomiR
  source 1..20: mol_type = other RNA
caattacgattatacatcct

SEQ ID NO: 11 - RNA, 21 nt, synthetic construct
  misc_feature 1..21: note = Description of sequence: miR-155-3p isomiR
  source 1..21: mol_type = other RNA
acaattacgattatacatcct

SEQ ID NO: 12 - RNA, 19 nt, synthetic construct
  misc_feature 1..19: note = Description of sequence: miR-155-3p isomiR
  source 1..19: mol_type = other RNA
actacgattatacatcctc

SEQ ID NO: 13 - RNA, 19 nt, synthetic construct
  misc_feature 1..19: note = Description of sequence: miR-155-3p isomiR
  source 1..19: mol_type = other RNA
aattacgattatacatcct

SEQ ID NO: 14 - RNA, 19 nt, synthetic construct
  misc_feature 1..19: note = Description of sequence: miR-155-3p isomiR
  source 1..19: mol_type = other RNA
attacgattatacatcctc

SEQ ID NO: 15 - RNA, 17 nt, synthetic construct
  misc_feature 1..17: note = Description of sequence: miR-155-3p isomiR
  source 1..17: mol_type = other RNA
acgattatacatcctca

SEQ ID NO: 16 - RNA, 18 nt, synthetic construct
  misc_feature 1..18: note = Description of sequence: miR-155-3p isomiR
  source 1..18: mol_type = other RNA
acgattatacatcctcag

SEQ ID NO: 17 - RNA, 20 nt, synthetic construct
  misc_feature 1..20: note = Description of sequence: miR-155-3p isomiR
  source 1..20: mol_type = other RNA
caatacgattatacatcctc

SEQ ID NO: 18 - RNA, 20 nt, synthetic construct
  misc_feature 1..20: note = Description of sequence: miR-155-3p isomiR
  source 1..20: mol_type = other RNA
attacgattatacatcctca

SEQ ID NO: 19 - RNA, 19 nt, synthetic construct
  misc_feature 1..19: note = Description of sequence: miR-155-3p isomiR
  source 1..19: mol_type = other RNA
cttacgattatacatcctc

SEQ ID NO: 20 - RNA, 18 nt, synthetic construct
  misc_feature 1..18: note = Description of sequence: miR-155-3p isomiR
  source 1..18: mol_type = other RNA
acaattacgattatacat

SEQ ID NO: 21 - RNA, 18 nt, synthetic construct
  misc_feature 1..18: note = Description of sequence: miR-155-3p isomiR
  source 1..18: mol_type = other RNA
ttacgattatacatcctc

(19) United States
(12) Patent Application Publication
(10) Pub. No.: US 2026/0256755 A1
(43) Pub. Date: Sep. 3, 2026

(54) COMBINATIONS FOR TREATMENT OF PARKINSON'S DISEASE AND OTHER PRIMARY AND SECONDARY PARKINSONIAN DISORDERS

(71) Applicant: Kenai Therapeutics, Inc., Cambridge, MA (US)

(72) Inventor: Howard J. Federoff, Cambridge, MA (US)

(73) Assignee: Kenai Therapeutics, Inc., Cambridge, MA (US)

(21) Appl. No.: 19/149,473

(22) PCT Filed: Feb. 26, 2024
(86) PCT No.: PCT/US2024/017335

§ 371 Date: Jul. 18, 2025

Related U.S. Application Data
(60) Provisional application No. 63/487,171, filed on Feb. 27, 2023.

(51) Int. Cl. A61K 31/444; A61K 31/216; A61K 35/30; A61K 45/06; A61P 25/00; A61P 29/00; A61P 43/00

(52) U.S. Cl. CPC A61K 31/444; A61K 31/216; A61K 35/30; A61K 45/06; A61P 25/00; A61P 29/00; A61P 43/00

(57) ABSTRACT

Disclosed are novel strategies for the treatment of patients with Parkinson's disease and other primary and secondary Parkinsonian disorders by enhancing cell engraftment. Cell viability, engraftment, proliferation, migration, or differentiation of administered DA neuronal cells is enhanced by treating the patient with an antilipemic agent and/or a CSF-1R antagonist before, during and/or after transplantation of DA neuronal cells.

### CROSS-REFERENCE TO RELATED APPLICATIONS

This application claims the benefit of U.S. Provisional Application No. 63/487,171, filed on Feb. 27, 2023 and entitled “COMBINATIONS FOR TREATMENT OF PARKINSON'S DISEASE AND OTHER PRIMARY AND SECONDARY PARKINSONIAN DISORDERS,” the entire contents of which are incorporated by reference herein.

### TECHNICAL FIELD

The invention relates to novel strategies for the treatment of patients with Parkinson's disease and other primary, secondary Parkinsonian disorders and Parkinson plus disease of multiple system atrophy, progressive supranuclear palsy, cortical-basal ganglionic degeneration and dementia with Lewy Bodies.

### BACKGROUND OF THE INVENTION

### Microglia

Microglia, the only immune cell type found in the brain, account for ~10% of the brain cell composition and have essential brain homeostasis maintaining functions such as phagocytic waste removal, injury repair, and maintenance of proper neuronal networks. However, excessive microglia activation induces inflammation, generating reactive oxygen species (ROS) and proinflammatory cytokines such as TNF-α and IL-β. Chronic inflammation contributes to neuronal stress and neurodegeneration.

### Differentiation to Lineage Specific Cell Populations

Cell populations that retain the ability to differentiate into numerous specialized cell types are useful for developing large numbers of lineage specific differentiated cell populations. These cell populations that retain a capability for further differentiation into specialized cells contain pluripotent cells. Pluripotent cells may be from embryonic and/or nonembryonic stem cell origin.

These lineage specific differentiated cell populations are contemplated to find use in cell replacement therapies for patients with diseases resulting in a loss of function of a defined cell population. In addition to their direct therapeutic value, lineage specific differentiated cells are also valuable research tools for a variety of purposes including in vitro screening assays to identify, confirm, and test for specification of function or for testing delivery of therapeutic molecules to treat cell lineage specific disease.

Previously, embryonic stem cells (ESCs), somatic stem cells and induced pluripotent stem cells (iPSCs) were used as therapeutics and model systems for neurodegenerative diseases. Research and technological developments relating to directed differentiation of embryonic and somatic stem cells has taken place in the field of diseases of the central nervous system (CNS), such as for Huntington's disease (HD), Alzheimer's disease (AD), Parkinson's disease (PD), and Multiple sclerosis (MS). However, there has been limited demonstrated therapeutic benefit to date.

Therefore, there is a need for compositions and methods to obtain cell populations capable of being used both in research and as a therapeutic for treating diseases when patients have lost a particular function due to a loss of a specific cell population.

In the case of Parkinson's disease, for example, it is the loss of midbrain dopaminergic (DA) neurons that results in the appearance of disease symptoms. Thus, there is a need for methods of producing DA neuronal cells from pluripotent cells, since such cells could be used both therapeutically and in disease models, e.g., to identify new therapeutics for treatments for Parkinson's disease and other primary and secondary Parkinsonian disorders, including but not limited to idiopathic Parkinson's, Parkinson plus disease of multiple system atrophy, progressive supranuclear palsy, cortical-basal ganglionic degeneration and dementia with Lewy Bodies, vascular parkinsonism, drug-induced parkinsonism and non-idiopathic Parkinson's disease disorders including but not limited to Parkinson's due to mutations in the Parkin gene and other familial and genetic causes of the diseases.

Various efforts have been made to generate midbrain DA neurons from pluripotent cells. For example, methodologies for generating midbrain DA neurons from pluripotent cells typically require use of both LDN-193189, an inhibitor of BMP signaling (inhibits ALK 1/2/3/6, blocks SMAD 1/5/8), and SB-431542, an inhibitor of TGF-beta signaling (inhibits ALK 4/5/7, blocks SMAD 2/3), as described, e.g., U.S. Pat. No. 10,280,398, which is herein incorporated by reference in its entirety. Since these methods utilize the combination of two inhibitors of Small Mothers Against Decapetaplegic (SMAD) signaling, these methods are typically referred to as “dual SMAD inhibition”, or “dual SMADi.”

One method to make DA neurons using dual SMAD inhibition comprises differentiating pluripotent stem cells, comprising exposing a plurality of pluripotent stem cells to at least one inhibitor of TGFβ/Activin-Nodal signaling, at least one inhibitor of bone morphogenetic protein (BMP) signaling, at least two activators of Sonic hedgehog (SHH) signaling, such as purmorphamine and SHH C25II, and at least one inhibitor of glycogen synthase kinase 3β (GSK3β) signaling that activates wingless (Wnt) signaling, wherein the exposure of the cells to the at least one inhibitor of TGFβ/Activin-Nodal signaling and at least one inhibitor of BMP signaling begins on day 0, wherein said cells are exposed to the at least one inhibitor of GSK3β signaling on the third (3rd) day through the eleventh (11th) day from the initial exposure of the cells to the at least one inhibitor of TGFβ/Activin-Nodal signaling and the at least one inhibitor of BMP signaling in amounts effective to produce a cell population comprising at least about 10% differentiated cells expressing both forkhead box protein A2 (FOXA2) and LIM homeobox transcription factor 1 alpha (LMX1A).

Additionally, U.S. Pat. No. 10,858,625, which is herein incorporated by reference in its entirety, discloses another method to make DA neurons using dual SMAD techniques comprising contacting a plurality of pluripotent stem cells with at least one inhibitor of TGFβ/Activin-Nodal signaling; and contacting the cells with at least one activator of Sonic hedgehog (SHH) signaling, and at least one activator of wingless (Wnt) signaling to obtain a population of differentiated cells expressing forkhead box protein A2 (FOXA2) and LIM homeobox transcription factor 1 alpha (LMX1A), wherein the concentration of the at least one activator of Wnt signaling is increased during the contact with the cells, and wherein i) the concentration increase is initiated between about 2 days and about 6 days from the initial contact of the at least one activator of Wnt signaling with the cells and ii) the concentration of the at least one activator of Wnt signaling is increased by between about 250% and about 1800% of the initial concentration of the at least one activator of Wnt signaling contacted with the cells.

Additionally, U.S. Pat. No. 10,273,452, which is herein incorporated by reference in its entirety, discloses another method to make DA neurons using dual SMAD techniques comprising contacting a plurality of starting cells with an inhibitor of Small Mothers Against Decapentaplegic (SMAD) protein signaling (“the SMAD inhibitor”), wherein the starting cells are selected from the group consisting of multipotent cells, pluripotent cells, and a combination thereof, and contacting the cells with a bone morphogenetic protein (BMP); contacting the cells with a compound selected from the group consisting of BRL-54443, parthenolide, phenanthroline, and combinations thereof, and wherein the cells are contacted with the SMAD inhibitor and the BMP in an amount effective to induce detectable expression of SIX1 and PAX6 in the plurality of cells.

Methods to obtain enrich populations of midbrain dopaminergic (DA) neurons are described in U.S. Pat. No. 10,828,335, which is herein incorporated by reference in its entirety. Generally, the method comprises the steps of: (a) obtaining a population of pluripotent cells; (b) culturing the population of cells in media comprising: a BMP signaling inhibitor; a TGFβ signaling inhibitor; an activator of Sonic hedgehog (SHH) signaling; and an activator of Wnt signaling, an MEK inhibitor and optionally free of added FGF8; (c) transferring the cell population to a suspension culture in a media comprising a BMP signaling inhibitor; an activator of SHH signaling; and an activator of Wnt signaling, thereby forming cell aggregates, optionally comprising added FGF8; (d) dissociating cell aggregates and seeding the dissociated cells into a culture to provide a neural lineage cell population; (e) further differentiating the neuronal lineage cell population in a maturation media comprising neuronal maturation factors to generate a cell population which includes midbrain neuron cells; and (f) using a transgenic screenable or selectable marker under the control of a pan-neural promoter expressed by cells of the cell population, to provide an enriched population of midbrain DA neurons.

Methods to prepare pluripotent stem cells for neural differentiation are described in U.S. Pat. No. 9,487,752, which is herein incorporated by reference in its entirety. Generally, the method comprises: a) culturing a population of human pluripotent stem cells in a medium comprising transforming growth factor β (TGFβ) and basic fibroblast growth factor (bFGF) that maintains cell pluripotency; b) priming the pluripotent stem cells, prior to aggregate formation, in an adherent culture and in a serum-free culture medium essentially free of externally added TGFβ and bFGF and in the absence of murine feeder cells; wherein priming occurs for at least one day; and wherein the levels of TGFβ and bFGF are gradually reduced; c) forming aggregates from the cells in step b) in a suspension culture; and d) further differentiating the aggregates into a cell population comprising neural cells, thereby producing human neural cells.

Others have generated midbrain DA neurons from pluripotent cells using mono-SMAD inhibition (mono-SMADi). See e.g. U.S. Pat. No. 10,590,383 which is herein incorporated by reference in its entirety. Generally, the method comprises culturing human pluripotent cells in the presence of the following signaling modulators: (a) a single inhibitor of Small Mothers Against Decapentaplegic (SMAD) signaling, (b) at least one activator of Sonic hedgehog (SHH) signaling, and (c) at least one activator of wingless (Wnt) signaling; and culturing said cells in the presence of said modulators for a period of time sufficient to provide a cell composition comprising FOXA2+/LMX1+ cells; wherein the culturing does not comprise culturing the human pluripotent cells in the presence of a second inhibitor of Small Mothers Against Decapentaplegic (SMAD) signaling.

### BRIEF DESCRIPTION OF THE DRAWINGS

FIG. 1 a flow chart of an exemplary method of treatment disclosed herein.

FIG. 2 are reproductions of Formulae (I) and (II) from US Patent Publication number 20170081326A1.

FIG. 3 is a reproduction of Table 1 from US Patent Publication number 20170081326A1.

### SUMMARY OF THE INVENTION

### Methods

### Method of Treating a Patient to Enhance Engraftment of Administered Cells

Disclosed is a method for pre-treating a patient to enhance engraftment of administered cells. Disclosed is a method for pre-treating a patient to enhance engraftment of administered progenitor cells. Disclosed is a method for pre-treating a patient to enhance engraftment of administered DA neuronal cells.

In some embodiments, a method for enhancing the efficacy of progenitor cell therapy in a mammal by administering to the mammal an antilipemic agent and/or CSF-1R antagonist is provided. In some embodiments, an antilipemic agent and/or a CSF-1R antagonist is administered in combination with cell therapy, resulting in enhanced effects of cell therapy.

### Administering an Antilipemic Agent or a CSF-1R Antagonist to the Patient

Disclosed is a method for pre-treating a patient to enhance engraftment of cells by administering to the patient a therapeutically effective amount of an antilipemic agent or a CSF-1R antagonist. Disclosed is a method for pre-treating a patient to enhance engraftment of cells by administering to the patient a therapeutically effective amount of an antilipemic agent. Disclosed is a method for pre-treating a patient to enhance engraftment of cells by administering to the patient a therapeutically effective amount of fenofibrate. Disclosed is a method for pre-treating a patient to enhance engraftment of cells by administering to the patient a therapeutically effective amount of fenofibrate. Disclosed is a method for pre-treating a patient to enhance engraftment of cells by administering to the patient a therapeutically effective amount of CSF-1R antagonist. Disclosed is a method for pre-treating a patient to enhance engraftment of cells by administering to the patient a therapeutically effective amount of pexidartinib, also known as PLX3397. Disclosed is a method for pre-treating a patient to enhance engraftment of cells by administering to the patient a therapeutically effective amount of PLX5622. The administration of the antilipemic agent or the CSF-1R antagonist can be before, during and after administering the cells to the patient for engraftment. In some embodiments, the cells for engraftment are stem cells, precursor cells, progenitor cells. In some embodiments, the cells for engraftment are DA neuronal cells.

### Administering an Antilipemic Agent and a CSF-1R Antagonist to the Patient

Disclosed is a method for pre-treating a patient to enhance engraftment of cells by administering to the patient a therapeutically effective amount of an antilipemic agent and CSF-1R antagonist. Disclosed is a method for pre-treating a patient to enhance engraftment of cells by administering to the patient a therapeutically effective amount of a fenofibrate and a CSF-1R antagonist. Disclosed is a method for pre-treating a patient to enhance engraftment of cells by administering to the patient a therapeutically effective amount of fenofibrate and a CSF-1R antagonist. Disclosed is a method for pre-treating a patient to enhance engraftment of cells by administering to the patient a therapeutically effective amount of fenofibrate and pexidartinib. Disclosed is a method for pre-treating a patient to enhance engraftment of cells by administering to the patient a therapeutically effective amount of fenofibrate and pexidartinib. The administration of the antilipemic agent and the CSF-1R antagonist can be before, during and after administering the cells to the patient for engraftment. In some embodiments, the cells for engraftment are progenitor cells. In some embodiments, the cells for engraftment are DA neuronal cells.

### Administering an Antilipemic Agent and a CSF-1R Antagonist to the Patient Before Administering Cell Therapy

In one embodiment, the method comprises identifying a mammal having a tissue with impaired function, providing the mammal with an antilipemic agent and/or CSF-1R antagonist, administering one or more cells to the tissue after administering the mammal with the an antilipemic agent and/or CSF-1R antagonist, wherein the an antilipemic agent and/or CSF-1R antagonist enhances one or more of the viability, engraftment, proliferation, migration, innervation or differentiation of the administered cells, thereby enhancing the efficacy of the cell therapy. In some embodiments, the administered cells are progenitor cells. In some embodiments, the administered cells are DA neuronal cells.

### Administering an Antilipemic Agent and a CSF-1R Antagonist to the Patient During Cell Therapy Administration

In one embodiment, the method comprises identifying a mammal having a tissue with impaired function, administering one or more cells to the tissue and simultaneously providing the mammal with an antilipemic agent and/or CSF-1R antagonist, wherein the antilipemic agent and/or CSF-1R antagonist enhances one or more of the viability, engraftment, proliferation, migration, innervation or differentiation of the administered cells, thereby enhancing the efficacy of the cell therapy. In some embodiments, the administered cells are progenitor cells. In some embodiments, the administered cells are DA neuronal cells.

Administering an Antilipemic Agent and a CSF-1R Antagonist to the Patient after Administering Cell Therapy

In one embodiment, the method comprises identifying a mammal having a tissue with impaired function, administering one or more cells to the tissue, then after administering the cells, providing the mammal with an antilipemic agent and/or CSF-1R antagonist, wherein the antilipemic agent and/or CSF-1R antagonist enhances one or more of the viability, engraftment, proliferation, migration, innervation or differentiation of the administered cells, thereby enhancing the efficacy of the cell therapy. In some embodiments, the administered cells are progenitor cells. In some embodiments, the administered cells are DA neuronal cells.

Administering an Antilipemic Agent and a CSF-1R Antagonist to the Patient Before, During, and after Administering Cell Therapy

In one embodiment, the method comprises identifying a mammal having a tissue with impaired function, administering one or more cells to the tissue, providing the mammal with an antilipemic agent and/or CSF-1R antagonist before, during, and after administering the cells, wherein the an antilipemic agent and/or CSF-1R antagonist enhances one or more of the viability, engraftment, proliferation, migration, innervation or differentiation of the administered cells, thereby enhancing the efficacy of the progenitor cell therapy. In some embodiments, the administered cells are progenitor cells. In some embodiments, the administered cells are DA neuronal cells. All combinations are contemplated: providing the mammal with an antilipemic agent and/or CSF-1R antagonist before and after (but not during) administering the cells or before and during (but not after) administering the cells, or during and after (but not before) administering the cells.

### Implantation of Treated Cells

Stem Cells are Treated with an Antilipemic Agent and/or a CSF-1R Antagonist In Vitro

In some embodiments, stem cells are treated with an antilipemic agent and/or a CSF-1R antagonist, differentiated to DA neuronal cells and then the DA neuronal cells are implanted into the brain of a patient. In some embodiments, the patient is at risk for Parkinson's disease, exhibits symptoms of Parkinson's disease, and/or has been diagnosed with Parkinson's disease.

The transplanted cells help repair damage to the brain or spinal cord or nerve(s) such that the recovery or prognosis is enhanced in patients having implanted progenitor cells as compared with those who do not receive such implants. In some embodiments, treatment of the stem cells with an antilipemic agent and/or a CSF-1R antagonist occurs one or more times in vitro prior to implantation.

In some embodiments, following implantation, the recipient site is treated with an antilipemic agent and/or a CSF-1R antagonist, including directly at the engraftment site, or some other portion of the brain or other neural tissue, such as the cortex or the spinal cord.

Progenitor Cells are Treated with an Antilipemic Agent and/or a CSF-1R Antagonist In Vitro

In some embodiments, cells differentiating to progenitor cells are treated with an antilipemic agent and/or a CSF-1R antagonist, once differentiated to DA neuronal cells, the DA neuronal cells are implanted or transplanted in the brain of a patient.

In some embodiments, progenitor cells are treated with an antilipemic agent and/or a CSF-1R antagonist and implanted or transplanted in the brain of a patient. In some embodiments, the patient is at risk for Parkinson's disease or other Parkinsonian disorders, exhibits symptoms of Parkinson's disease or other Parkinsonian disorders, and/or has been diagnosed with Parkinson's disease or other Parkinsonian disorders. The transplanted cells help repair damage to the spinal cord or nerve(s) such that the recovery or prognosis is enhanced in patients having implanted progenitor cells as compared with those who do not receive such implants. In some embodiments, treatment of the progenitor cells with an antilipemic agent and/or a CSF-1R antagonist occurs one or more times in vitro prior to implantation. In some embodiments, the transplanted cells produce dopamine to treat, or lessen the symptoms and/or delay onset of Parkinson's disease in the patient.

Progenitor Cells are Treated with an Antilipemic Agent and/or a CSF-1R Antagonist In Vivo

In some embodiments, the progenitor cells are treated with an antilipemic agent and/or a CSF-1R antagonist after delivery to the target tissue (e.g., in vivo). In some embodiments, this approach improves the overall efficacy of treatment, as there is limited lag time between the exposure of the cells to an antilipemic agent and/or a CSF-1R antagonist and the receipt of beneficial effects by the target tissue. In some embodiments, combinations of an antilipemic agent and/or a CSF-1R antagonist treatment are used. For example, in some embodiments, cells are treated with an antilipemic agent and/or a CSF-1R antagonist both before, during, after or combinations thereof administration. In some embodiments, treatment with an antilipemic agent and/or a CSF-1R antagonist occurs one or more times in vitro prior to implantation and/or one or more times after implantation.

### Compositions

### DA Neuronal Cell Populations

Disclosed is a DA neuronal cell population or populations, comprising, consisting essentially of, or consisting of an antilipemic agent or CSF-1R antagonist. A DA neuronal cell population comprising, consisting essentially of, or consisting of an antilipemic agent. A DA neuronal cell population comprising, consisting essentially of, or consisting of fenofibrate. A DA neuronal cell population comprising, consisting essentially of, or consisting of fenofibrate. A DA neuronal cell population comprising, consisting essentially of, or consisting of a CSF-1R antagonist. A DA neuronal cell population comprising, consisting essentially of, or consisting of pexidartinib.

Provided herein is a DA neuronal cell population or populations comprising, consisting essentially of, or consisting of an antilipemic agent and a CSF-1R antagonist. A DA neuronal cell population comprising, consisting essentially of, or consisting of fenofibrate and a CSF-1R antagonist. A DA neuronal cell population comprising, consisting essentially of, or consisting of fenofibrate and a CSF-1R antagonist. A DA neuronal cell population comprising, consisting essentially of, or consisting of fenofibrate and pexidartinib. A DA neuronal cell population comprising, consisting essentially of, or consisting of fenofibrate and pexidartinib.

For each of the above compositions, the compositions can be administered to subjects identified as likely to have a neurologic disease such as Parkinson's disease or other primary and secondary Parkinsonian disorders.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

The practice of the present disclosure employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)).

### General Discussion of Parkinson's Disease and Other Primary and Secondary Parkinsonian Disorders

Injury and/or disease can result in the loss of function or death of cells in a tissue afflicted with or indirectly impacted by the disease or injury. For example, age-related degeneration of tissues can lead to loss of function of neurons in the eye, loss of tactile sensations, reduced control over muscle movement, memory failure, among many other possible effects. Non-neural tissues are also subject to damage or disease. For example, cardiac tissue may be damaged after an adverse myocardial event, such as a myocardial infarction. Blood cells may be damaged by chemotherapy or radiation therapy. Liver cells may be damaged by toxins or metabolic waste by products. These diseases and/or injuries, among others, are all candidates for cell therapy.

Cell therapy, the introduction of new cells into a tissue in order to treat a disease, represents a possible method for repairing or replacing diseased tissue with healthy tissue. In some embodiments, described herein, antilipemic agent and/or CSF-1R antagonist, is used to augment the effects of cell therapy. As described in more detail below, an antilipemic agent and/or a CSF-1R antagonist is used in some embodiments (either by contacting transplanted cells or pre-treating the recipient of transplanted cells), to enhance the viability of transplanted cells. In some embodiments, enhanced viability is manifest as a more robust population of cells to transplant into a subject requiring cellular therapy. In some embodiments, use of an antilipemic agent and/or a CSF-1R antagonist (either by contacting transplanted cells or pre-treating the recipient of transplanted cells) results in transplanted cells which stabilize to a greater degree, have enhanced survival post-implantation, have increased engraftment, and the like. In some embodiments, use of an antilipemic agent and/or a CSF-1R antagonist (either by contacting transplanted cells or pre-treating the recipient of transplanted cells) enhances the activation and differentiation of endogenous stem cells. In some embodiments, an antilipemic agent and/or a CSF-1R antagonist is used to treat harvested stem cells (or cultured stem cells) prior to administration to an individual requiring therapy. In some embodiments, DA neuronal cells are administered and then an antilipemic agent and/or a CSF-1R antagonist is administered to the patient including within the transplanted region. In some embodiments, cells are administered without previously exposing the cells to an antilipemic agent and/or a CSF-1R antagonist (e.g., cells not exposed until after administration). In some embodiments, a target tissue/brain region is pre-treated with an antilipemic agent and/or a CSF-1R antagonist prior to administration of cells (which either have or have not yet been treated with an antilipemic agent and/or a CSF-1R antagonist). In some embodiments, an antilipemic agent and/or a CSF-1R antagonist is administered 10 days prior to administration of cells (which either have or have not yet been treated with an antilipemic agent and/or a CSF-1R antagonist). In some embodiments, the time period before cell administration is 15 days, 20 days, 30 days, 40 days, 50 days or 60 days. In some embodiments, an antilipemic agent and/or a CSF-1R antagonist is administered prior to administration of cells (which either have or have not yet been treated with an antilipemic agent and/or a CSF-1R antagonist), and then an antilipemic agent and/or a CSF-1R antagonist is re-administered post-cell administration for a period of days. In some cases, an antilipemic agent and/or a CSF-1R antagonist is administered for 10 days, 15 days, 20 days, 30 days, 2 months, 3 months, 4 months, 5 months or 6 months. In some embodiments, only an antilipemic agent such as fenofibrate is re-administered post-cell administration. In some embodiments, cells are treated with an antilipemic agent and/or a CSF-1R antagonist, then incubated for a period of time prior to administration. For example, the incubation period ranges from a one or more minutes to about 48 hours, in some embodiments. In some embodiments, the incubation period ranges from about 1 to about 5 minutes, about 5 to about 10 minutes, about 10 to about 15, minutes, about 15 to about 20 minutes, about 20 minutes to about 30 minutes, about 30 minutes to about 40 minutes, about 40 minutes to about 50 minutes, about 50 minutes to about 60 minutes, and overlapping ranges thereof. In some embodiments, the post-administration waiting period is from 1-4, 4-8, 8-12, 12-16, 16-20, 20-24 hours, and overlapping ranges thereof. Longer or shorter incubation periods are used in some embodiments. In some embodiments, cells are administered concurrently with an antilipemic agent and/or a CSF-1R antagonist administration to the target tissue. In some embodiments, cells are administered to a subject, a period of time elapses, and then an antilipemic agent and/or a CSF-1R antagonist is administered to the patient and/or directly to the target tissue. For example, the post-administration waiting period ranges from a one or more minutes to about 48 hours, in some embodiments. In some embodiments, the post-administration waiting period ranges from about 1 to about 5 minutes, about 5 to about 10 minutes, about 10 to about 15, minutes, about 15 to about 20 minutes, about 20 minutes to about 30 minutes, about 30 minutes to about 40 minutes, about 40 minutes to about 50 minutes, about 50 minutes to about 60 minutes, and overlapping ranges thereof. In some embodiments, the post-administration waiting period is from 1-4, 4-8, 8-12, 12-16, 16-20, 20-24 hours, and overlapping ranges thereof. Longer or shorter incubation periods are used in some embodiments. Administration of an aantilipemic agent and/or a CSF-1R antagonist in conjunction with DA neuronal cells can, in some embodiments, enhance the effects of the implanted cells and advantageous provides improved therapy for a wide variety of clinical applications.

### Definitions

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein the term “about” refers to +/−10%.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

“Formulae (I) or (II) or a pharmaceutically acceptable salt, a solvate, a tautomer, an isomer, or a deuterated analog of Formulae (I) or (II)” means the Formulae (I) or (II) as described in US Patent Publication number 20170081326A1 wherein for formula I, R1 is cyano, halo, or (C1-C3) alkyl optionally substituted with 1 to 3 substituents independently selected from the group consisting of halo, methyl, ethyl, methoxy and ethoxy; and X, when present, is halo and wherein for formula II, R¹ is cyano, halo, or (C₁-C₃)alkyl optionally substituted with 1 to 3 substituents independently selected from the group consisting of halo, methyl, ethyl, methoxy and ethoxy.

As used herein, the term “neurodegeneration” shall be given its ordinary meaning and shall also refer to the process of cell destruction resulting from primary destructive events such as stroke or trauma, and also secondary, delayed and progressive destructive mechanisms that are invoked by cells due to the occurrence of the primary destructive event. Primary destructive events include disease processes or physical injury or neurotoxin exposure or insult, including stroke, but also include other diseases and conditions such as multiple sclerosis, amyotrophic lateral sclerosis, heat stroke, epilepsy, Alzheimer's disease, Parkinson's disease, Huntington's disease, dopaminergic impairment, dementia resulting from other causes such as AIDS, cerebral ischemia including focal cerebral ischemia, and physical trauma such as crush or compression injury in the CNS, including a crush or compression injury of the brain, spinal cord, nerves or retina, or any other acute injury or insult producing neurodegeneration. Secondary destructive mechanisms include any mechanism that leads to the generation and release of neurotoxic molecules, including apoptosis, depletion of cellular energy stores because of changes in mitochondrial membrane permeability, release or failure in the reuptake of excessive glutamate, reperfusion injury, and activity of cytokines and inflammation. Both primary and secondary mechanisms may contribute to forming a “zone of danger” for neurons, wherein the neurons in the zone have at least temporarily survived the primary destructive event, but are at risk of dying due to processes having delayed effect.

As used herein, the term “neuroprotection” shall be given its ordinary meaning and shall also refer to a therapeutic strategy for slowing or preventing the otherwise irreversible loss of neurons due to neurodegeneration after a primary destructive event, whether the neurodegeneration loss is due to disease mechanisms associated with the primary destructive event or secondary destructive mechanisms.

As used herein, the term “composition” as used herein has its broadest reasonable meaning, including but not limited to a composition comprising, consisting of, or consisting essentially of a therapeutically effective amount of an antilipemic agent and/or a CSF-1R antagonist. In some embodiments, the composition further comprises DA neuronal cells.

As used herein, the term “combination therapy” as used herein has its broadest reasonable meaning, including but not limited to the process whereby a patient is treated with a therapeutically effective amount of an antilipemic agent and/or a CSF-1R antagonist and DA neuronal cells are delivered to the patient's target tissue.

As used herein, the term “cognitive function” as used herein shall be given its ordinary meaning and shall also refer to cognition and cognitive or mental processes or functions, including those relating to knowing, thinking, learning, perception, memory (including immediate, recent, or remote memory), and judging. Symptoms of loss of cognitive function can also include changes in personality, mood, and behavior of the patient. Diseases or conditions affecting cognitive function include Alzheimer's disease, dementia, AIDS or HIV infection, Cruetzfeldt-Jakob disease, head trauma (including single-event trauma and long-term trauma such as multiple concussions or other traumas which may result from athletic injury), Lewy body disease, Pick's disease, Parkinson's disease, Lewy Body Dementia, frontotemporal dementia (FTD), Huntington's disease, drug or alcohol abuse, brain tumors, hydrocephalus, kidney or liver disease, stroke, depression, and other mental diseases which cause disruption in cognitive function, and neurodegeneration.

“DA neuronal cells” or “DA neurons” are cells of the midbrain fate that express FOXA2+, LMX1A+, and TH+.

As used herein, the term “differentiation” refers to a process whereby an unspecialized embryonic cell acquires the features of a specialized cell such as a specific type of neuron, brain cell, heart, liver, or muscle cell. Differentiation is controlled by the interaction of a cell's genes with the physical and chemical conditions outside the cell, usually through signaling pathways involving proteins embedded in the cell surface.

As used herein, the term “cell differentiation” refers to a pathway by which a less specialized cell (i.e. stem cell) develops or matures to possess a more distinct form and function (for example, an iPSC progressing into a neural crest progenitor to a cell of neuronal lineage to a floor plate midbrain progenitor cells to a midbrain fate FOXA2/LMX1A+ dopamine (DA) neurons).

The term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vivo, treatment and/or mutagenesis. A cell may be derived from another cell, using for example chemical treatment, radiation, inducing new protein expression, for example, by infection with virus, transfection with DNA/RNA sequences, contacting (treating) with a morphogen, etc., and selection (such as by serial culture) of any cell type that is contained in cultured parent cells). A derived cell can be selected from a mixed population by virtue of response to a growth factor, cytokine, selected progression of cytokine treatments, adhesiveness, lack of adhesiveness, sorting procedure, and the like.

As used herein, the term “directed differentiation” refers to a manipulation of stem cell culture conditions to induce differentiation into a particular (for example, desired) cell type, such as floor plate midbrain progenitor cells and midbrain fate FOXA2/LMX1A+ dopamine (DA) neurons. In one embodiment, the term “directed differentiation” in reference to a cell refers to the use of small molecules, growth factor proteins, and other growth conditions to promote the transition of a cell from a pluripotent state into a more mature or specialized cell fate (e.g. central nervous system cell, neural cell, floor plate midbrain progenitor cell and midbrain fate FOXA2/LMX1A+ dopamine (DA) neuron, etc.). In one preferred embodiment, the beginning of directed differentiation is the contacting of a cell at day 0 with LDN/SB. A cell undergoing directed differentiation as described herein results in the formation of a non-default cell type of floor plate midbrain progenitor cells and midbrain fate FOXA2/LMX1A+ dopamine (DA) neurons.

The term “differentiation” as used herein has its broadest reasonable meaning, including but not limited to the process whereby an unspecialized, pluripotent stem cell proceeds through one or more intermediate stage cellular divisions, and in some cases ultimately producing one or more specialized cell types. Differentiation thus includes the process whereby precursor cells, e.g., uncommitted cell types that precede the fully differentiated forms but may or may not be true stem cells, proceed through intermediate stage cell divisions to ultimately produce specialized cell types. Differentiation encompasses the process whereby pluripotent stem cells are induced to differentiate into DA neuronal cells. Differentiation encompasses the process whereby pluripotent stem cells are induced to differentiate into cell types comprising the central nervous system, in vivo or in vitro.

The term “Embryonic stem (ES) cells” means pluripotent stem cells derived from early embryos. The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763) and embryonic germ (EG) cells which are obtained from the genital tissue of a fetus any time during gestation, preferably before 10 weeks of gestation.

As used herein, the term “engraftment” shall be given its ordinary meaning and shall also refer to the process (or result of that process) whereby a cell is incorporated into another group of cells or another tissue. For example, in some embodiments, exogenously administered cells engraft (e.g., become a part of) the host neural network. Engraftment may or may not occur in conjunction with migration, depending on the embodiment. Likewise, engraftment may or may not be associated with increased viability (e.g., engraftment is not a requirement for maintaining or increasing viability of cells), depending on the embodiment.

The terms “growth chamber” and “cell culture chamber” as used herein are used interchangeably and are to be interpreted very broadly to refer to any container or vessel suitable for culturing cells, including, but not limited to, dishes, culture plates (single or multiple well), bioreactors, incubators, and the like.

The term “Induced pluripotent stem cells” commonly abbreviated as “iPS cells” or “iPSCs,” refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing or contacting with reprogramming factors. Induced pluripotent stem cells are endowed with pluripotency (i.e., being capable of differentiating into the three embryonic germ cell layers, i.e., endoderm, ectoderm and mesoderm). According to some embodiments, the induced pluripotent stem cells are formed by inducing the expression of Oct-4.

The term “migration” as used herein shall be given its ordinary meaning and shall also refer to the movement of a stem cell (either endogenous or exogenous) from its initial site (e.g., an endogenous storage site or a site of administration) to a second site (e.g., a final position in a target tissue). In some embodiments, migration occurs based on fluid flow or pressure changes in the environment surrounding the cell. In some embodiments, chemoattractant or chemorepellents induce migration of cells.

The terms “Neurons” or “neural cells” or “neural cell types” or “neural lineage” may include any neuron lineage cells, and can be taken to refer to cells at any stage of neuronal ontogeny without any restriction, unless otherwise specified. For example, neurons may include both neuron precursor cells, mature neurons and cell types originating from neuronal common progenitor domain such as astrocytes.

As used herein, the term “neural lineage cell” refers to a cell that contributes to the nervous system (both central and peripheral) or neural crest cell fates during development or in the adult. The nervous system includes the brain, spinal cord, and peripheral nervous system. Neural crest cell fates include cranial, trunk, vagal, sacral, and cardiac, giving rise to mesectoderm, cranial cartilage, cranial bone, thymus, teeth, melanocytes, iris pigment cells, cranial ganglia, dorsal root ganglia, sympathetic/parasympathetic ganglia, endocrine cells, enteric nervous system, and portions of the heart.

The term “parkinsonism” refers to a group of diseases that are all linked to an insufficiency of dopamine in the basal ganglia which is a part of the brain that controls movement. Symptoms include tremor, bradykinesia (extreme slowness of movement), flexed posture, postural instability, and rigidity. A diagnosis of parkinsonism requires the presence of at least two of these symptoms, one of which must be tremor or bradykinesia. The most common form of parkinsonism is idiopathic, or classic, Parkinson's disease (PD), but for a significant minority of diagnoses, about 15 percent of the total, one of the Parkinson's plus syndromes (PPS) may be present. These syndromes also known as atypical parkinsonism, include cortico-basal degeneration, Lewy body dementia, multiple system atrophy, and progressive supranuclear palsy. In general, Parkinson's disease involves the malfunction and death of vital nerve cells in the brain primarily in an area of the brain called the substantia nigra. Many of these vital nerve cells make dopamine, that as these neurons die off, the amount of dopamine resulting from synthesis in the brain decreases, leaving a person unable to control movement normally. The intestines also have dopamine cells that degenerate in Parkinson's disease patients, and this may be an important causative factor in the gastrointestinal symptoms that are part of the disease. A group of symptoms that an individual experiences varies from person to person. Primary motor signs of Parkinson's disease include the following: tremor of the hands, arms, legs, jaw and face, bradykinesia or slowness of movement, rigidity or stiffness of the limbs and trunk and postural instability or impaired balance and coordination.

The term “prior art preparation” means any pharmaceutical composition as described in WO2014145051A1, US Patent Publication number 20170081326A1 and U.S. Pat. No. 10,717,735.

The terms “Pluripotency” or “pluripotent” refers to a stem cell or undifferentiated cell that has the potential to differentiate into all cells constituting one or more tissues or organs, for example, any of the three germ layers: endoderm (interior stomach lining, gastrointestinal tract, the lungs), mesoderm (muscle, bone, blood, urogenital), or ectoderm (epidermal tissues and nervous system).

The term “precursor cell” as used herein has its broadest reasonable meaning, including but not limited to a pluripotent, or lineage-uncommitted, progenitor cell, a “stem cell” or “mesenchymal stem cell” (MSC), that is potentially capable of an unlimited number of mitotic divisions to either renew its line or to produce progeny cells that will differentiate into any of a variety of cells (e.g., cells of the central nervous system including neural cells such as astrocytes, oligodendrocytes, and neurons; cardiac cells; hematopoietic cells, etc.).

The term “progenitor cell” as used herein has its broadest reasonable meaning, including but not limited to a lineage-committed precursor cell produced from the mitotic division of a stem cell which will eventually differentiate into a neural cell (or other cell type within the lineage of the stem cell, e.g., a cardiac progenitor cell differentiating into a cardiomyocyte). Unlike the stem cell from which it is derived, a progenitor cell is generally considered to be incapable of an unlimited number of mitotic divisions and will eventually differentiate into a cell type within its lineage (e.g., neural to neural, cardiac to cardiac, etc.).

As used herein, the term “proliferation” shall be given its ordinary meaning and shall refer to the process by which one or more stem cells (endogenous or exogenous) divide and increase the population of stem cells (e.g., mitotic division). In some embodiments, proliferation is measured by simple total cell count. In other embodiments, proliferation is assessed by expression of certain proteins (e.g., proliferating cell nuclear antigen, PCNA), or by monitoring entry of cells into the cell cycle.

As used herein, the term “motor function” as used herein shall be given its ordinary meaning and shall also refer to those bodily functions relating to muscular movements, primarily conscious muscular movements, including motor coordination, performance of simple and complex motor acts, and the like.

As used herein, the term “neurologic function” as used herein shall be given its ordinary meaning and shall also refer to both cognitive function and motor function.

As used herein, the terms “cognitive enhancement” and “motor enhancement” as used herein shall be given its ordinary meaning and shall also refer to the improving or heightening of cognitive function and motor function, respectively.

As used herein, the term “neurologic enhancement” as used herein shall be given its ordinary meaning and shall also include both cognitive enhancement and motor enhancement.

As used herein, the term “neuroprotective effective” as used herein shall be given its ordinary meaning and shall also refer to the amount of DA neuronal cells and an antilipemic agent and/or a CSF-1R antagonist to achieve the goal of preventing, avoiding, reducing or eliminating neurodegeneration, which should result in cognitive enhancement and/or motor enhancement.

As used herein, the term “neurologic function enhancement effective” as used herein shall be given its ordinary meaning and shall also refer to the amount of DA neuronal cells and an antilipemic agent and/or a CSF-1R antagonist to achieve the goal of neuroprotection, motor enhancement and/or cognitive enhancement, and/or enhancement of stem cell viability, proliferation, differentiation, or increased efficacy of cell therapy.

As used herein, “pharmaceutically acceptable salt thereof” includes an acid addition salt or a base salt.

As used herein, “pharmaceutically acceptable carrier” includes any material which, when combined with a composition disclosed herein, allows the composition to retain biological activity, such as the ability to treat inflammation associated disease or affect the various mechanisms associated therewith, and is non-reactive with the subject's immune system. Examples include, but are not limited to, any of the standard pharmaceutical carriers such as a phosphate buffered saline solution, water, emulsions such as oil/water emulsions, and various types of wetting agents. Compositions comprising such carriers are formulated by well-known conventional methods (see, for example, Remington's Pharmaceutical Sciences, Chapter 43, 14th Ed., Mack Publishing Co., Easton, Pa.).

As used herein, the term “Sma Mothers Against Decapentaplegic” or “Small Mothers Against Decapentaplegic” or “SMAD” refers to a signaling molecule.

As used herein, the term “stem cell” refers to a cell with the ability to divide for indefinite periods in culture and to give rise to specialized cells. A stem cell may be obtained from animals and patients, including humans; for example, a human stem cell refers to a stem cell that is human. A stem cell may be obtained from a variety of sources including embryonic and nonembryonic, such as umbilical cord cells, cells from children and cells from adults. Adult stem cells in general refer to cells that were not originally obtained from a fetus, in other words, cells from babies, cast off umbilical cords, cast off placental cells, cells from children, cells from adults, etc.

The terms “subject,” “individual” or “patient” are used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

The term or phrase “transplantation”, “cell replacement”, “administering” or “grafting” are used interchangeably herein and refer to the introduction of the cells disclosed herein to target tissue. As mentioned, the cells can be derived from the recipient or from an allogeneic, semi-allogeneic or xenogeneic donor.

As used herein, the term “therapeutic agent” means an agent that has a therapeutic effect in, for example, ameliorating or treating a neurodegenerative disorder or damage.

As used herein, the term “viability” shall be given its ordinary meaning and shall also refer to the ability of a cell, be it a stem cell or a resident cell, to survive disease, trauma, or other injury that would compromise the normal functionality of the cell. In some embodiments, viability is measured by assessing the size of a certain population of cells, while in some embodiments, specific chemical, biological, or analytical tests are performed to evaluate the viability of the cells. Viability is also, in some embodiments, assessed by function, wherein an increase in function may be associated with an increase in viability.

“Therapeutically effective amount” means a dose that causes a targeted effect of administration, preferably such effect is enhancing the efficacy of the cell therapy.

“miR-155-3p biased or miR-155-5p biased” means the cell population is biased for miR-155-3p or miR-155-5p. For example, (1) it expresses more miR-155-3p compared to miR-155-5p, or (2) it expresses more miR-155-3p/miR-155-5p compared to native cells.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

Unless otherwise defined, all technical and/or scientific terms used herein have the same meaning as commonly understood by one of ordinary skill in the art. Although methods and materials similar or equivalent to those described herein can be used in the practice or testing of embodiments disclosed herein, exemplary methods and/or materials are described below. In case of conflict, the patent specification, including definitions, will control. In addition, the materials, methods, and examples are illustrative only and are not intended to be necessarily limiting.

Before explaining at least one embodiment in detail, it is to be understood that the disclosure is not necessarily limited in its application to the details set forth in the following description or exemplified by the Examples. The disclosure is capable of other embodiments or of being practiced or carried out in various ways.

### Antilipemic Agents

Antilipemic agents, also known as antihyperlipidemic agents, which may be utilized with the methods and compositions described herein are bile acid sequestrants, fenofibrate derivatives, HMG-CoA reductase inhibitors and nicotinic acid compounds. Antilipemic agents reduce the amount of cholesterol and fats in the blood through a number of mechanisms. For example, bile acid sequestrants bind to bile acids in the intestine and prevent them from being reabsorbed into the blood. The liver then produces more bile to replace the bile which has been lost. Since the body needs cholesterol to make bile, the liver uses up the cholesterol in the blood, reducing the amount of LDL cholesterol circulating in the blood.

Fenofibrate derivatives which may be used in the disclosed combination include, but are not limited to, clifofibrate, pirifibrate, ciprofibrate, bezafibrate, clinofibrate, ronifibrate, theofibrate, clofibrate, etofibrate, gemfibrozil and fenofibrate. HMG-CoA reductase inhibitors, also known as statins, include cerivastatin, fluvastatin, atorvastatin, lovastatin, pravastatin and simvastatin, or the pharmaceutically acceptable salt forms thereof. Niacin is an example of a nicotinic acid compound which may be used with the disclosed methods. Also useful are lipase inhibiting agents, such as orlistat. The use of these agents is described in further detail in U.S. Patent Pub. No. 2002/0198202-A1, which is herein incorporated by reference in its entirety.

Clifofibrate is commercially available in the form of 500 mg ATROMID-S® capsules from Wyeth-Ayerst Pharmaceuticals, with a recommended daily dosage of about 2 g administered in divided doses. Gemfibrozoil is available in 600 mg LOPID® tablets from Parke-Davis, with a recommended dose for adults of about 1200 mg per day administered in two divided doses 30 minutes prior to the morning and evening meals. Fenofibrate is available in 67 mg, 134 mg and 200 mg TRICOR® tablets from Abbott Laboratories Inc., with a recommended initial dose of from 67 mg to 200 mg per day, up to a maximum daily dose of 200 mg per day.

In some embodiments, more than one antilipemic agent is administered to the patient to promote cell viability, engraftment, proliferation, migration, or differentiation of administered DA neuronal cells.

Surprisingly, Applicant discovered that only fenofibrate and not its primary metabolite fenofibric acid are active. Specifically, both clorofibrate and benzafibrate are unable to upregulate PGC-1a, the new target and mode of action relevant to dopaminergic cells and Parkinson's disease.

### CSF-1R Antagonist

CSF-1R antagonists which may be used in the disclosed combination include, but are not limited to, axitinib (AG 013736), dasatinib (BMS 354825), erlotinib, gefitinib, flavopiridol, imatinib mesylate, lapatinib, motesanib diphosphate (AMG 706), nilotinib (AMN107), seliciclib, sorafenib, sunitinib malate, AEE-788, BMS-599626, UCN-01 (7-hydroxystaurosporine), vemurafenib, dabrafenib, selumetinib, LGX818, BGB-283, pexidartinib (PLX3397), PLX5622 and vatalanib. In some embodiments, the CSF-1R antagonists which may be used in the disclosed combination are pexidartinib (PLX3397) and PLX5622.

In some embodiments, more than one CSF-1R antagonist is administered to the patient to promote cell viability, engraftment, proliferation, migration, innervation or differentiation of administered DA neuronal cells.

### Possible Action Mechanisms

### Fenofibrate

The following section discusses theories and potential action mechanisms, as they presently appear to the inventors, for certain embodiments described herein. The scope of the claims of the present application is not to be construed to depend on the accuracy, relevance, or specifics of any of these theories or potential action mechanisms. Thus the claims of the present application are to be construed without being bound by theory or by a specific mechanism.

Fenofibrate promotes effective mitochondrial function, it promotes anti-inflammation, and provides general neuroprotection. Fenofibrate can restore the ability of PGC1-alpha to turn mitochondrial biogenesis back on and promote effective mitophagy. Fenofibrate has been used to treat traumatic brain injury, treat animal models of AD, and treat animal models of PD. Generally speaking, fenofibrate is used to modify disease progression. In contrast here, fenofibrate is used to promote cell engraftment. The literature has not described DA neural cell/fenofibrate co-administration (antecedent, concurrent, and/or post-engraftment) to promote DA neuronal progenitor survival after engraftment.

Furthermore, while fenofibrate has been shown to have an anti-inflammatory effect, it has never been shown to affect stem cells or DA neuronal progenitor survival after engraftment.

It is expected that fenofibrate, after administration, will be converted to its active metabolite, fenofibric acid. Fenofibric acid will ligate the receptor PPAR-alpha, turning on a number of genes that will promote an anti-inflammatory effect. Indeed, the FDA approved fenofibrate as a prodrug that, when metabolized to fenofibric acid, can ligate PPARalpha and reduce triglycerides. Applicant surprisingly discovered that fenofibrate is active on dopaminergic stem cells and the brain environment in which they are placed. Thus, the Applicant discovered a new mechanism of action of fenofibrate that is directly relevant to promoting mitochondrial homeostasis in engrafted dopaminergic progenitors (DA neuronal cells).

Aspects and embodiments of the methods disclosed herein can be further understood by in the following numbered paragraphs:

Paragraph 1. A composition for improving cell therapy in a subject, wherein the composition consists of a therapeutically effective amount of an antilipemic agent.

Paragraph 2. A composition for improving cell therapy in a subject, wherein the composition consists of a therapeutically effective amount of fenofibrate.

Paragraph 2a. A composition for improving cell therapy in a subject, wherein the composition consists of a therapeutically effective amount of fenofibrate and not clorofibrate and benzafibrate

Paragraph 2b. A composition for improving cell therapy in a subject, wherein the composition consists of a therapeutically effective amount of fenofibrate and not clorofibrate, benzafibrate and/or fenofibric acid.

Paragraph 3. A composition for improving cell therapy in a subject, wherein the composition consists of a therapeutically effective amount of fenofibrate.

Paragraph 4. A composition for improving cell therapy in a subject, wherein the comprises is a therapeutically effective amount of an antilipemic agent.

Paragraph 5. A composition for improving cell therapy in a subject, wherein the comprises is a therapeutically effective amount of fenofibrate.

Paragraph 6. A composition for improving cell therapy in a subject, wherein the comprises is a therapeutically effective amount of fenofibrate.

Paragraph 7. The composition of any preceding paragraph wherein the composition does not comprise any prior art preparations.

Paragraph 8. The composition of any preceding paragraph wherein the composition further comprises at least one pharmaceutically acceptable carrier.

Paragraph 9. The composition of any preceding paragraph wherein the composition further comprises at least one additional therapeutic agent.

Paragraph 10. The composition of any preceding paragraph wherein the composition further comprises a CSF-1R antagonist.

Paragraph 11. The composition of any preceding paragraph wherein the composition further comprises DA neuronal cells.

Paragraph 12 The composition of any preceding paragraph wherein the composition further comprises DA neuronal cells and stem cells.

Paragraph 13. The composition of any preceding paragraph wherein the composition further comprises cells for administration.

Paragraph 14. A kit comprising the composition of any preceding paragraph.

Paragraph 13. The composition of any preceding paragraph comprising a pharmaceutically acceptable salt of paragraphs 1-13.

### Pexidartinib

Pexidartinib, or PLX3397, was shown to inhibit the survival of microglia and cause a fast depletion of the microglia population in the healthy brain, thus also suggesting its application in terms of being capable of resetting the microglial imbalance that occurs in the inflamed brain. This also suggests that any microglia that are renewed following Pexidartinib administration will not be polarized. It is expected, therefore, that antecedent or concurrent administration of Pexidartinib will facilitate an in-vivo environment where later administration of a neural cell engraftment can then influence the magnitude, order and specificity of microglia formation post-engraftment.

In some embodiments, the exposure of the patient to Pexidartinib will be shorter than exposure to fenofibrate.

In some embodiments, the exposure of the patient to fenofibrate will be shorter than exposure to Pexidartinib.

The literature has not described DA neural cell/Pexidartinib co-administration (antecedent, concurrent, and post-engraftment) to promote DA neuronal progenitor survival after engraftment.

Aspects and embodiments of the methods disclosed herein can be further understood by in the following numbered paragraphs:

Paragraph 1. A composition for improving cell therapy in a subject, wherein the composition consists of a therapeutically effective amount of a CSF-1R antagonist.

Paragraph 2. A composition for improving cell therapy in a subject, wherein the composition consists of a therapeutically effective amount of pexidartinib.

Paragraph 2a. A composition for improving cell therapy in a subject, wherein the composition consists of a therapeutically effective amount of pexidartinib and not clorofibrate and benzafibrate.

Paragraph 2b. A composition for improving cell therapy in a subject, wherein the composition consists of a therapeutically effective amount of pexidartinib and not clorofibrate, benzafibrate and/or fenofibric acid.

Paragraph 3. A composition for improving cell therapy in a subject, wherein the comprises is a therapeutically effective amount of a CSF-1R antagonist.

Paragraph 4. A composition for improving cell therapy in a subject, wherein the comprises is a therapeutically effective amount of pexidartinib.

Paragraph 5. The composition of any preceding paragraph wherein the composition does not comprise any prior art preparations.

Paragraph 6. The composition of any preceding paragraph wherein the composition further comprises at least one pharmaceutically acceptable carrier.

Paragraph 7. The composition of any preceding paragraph wherein the composition further comprises at least one additional therapeutic agent.

Paragraph 8. The composition of any preceding paragraph wherein the composition further comprises an antilipemic agent.

Paragraph 9. The composition of any preceding paragraph wherein the composition further comprises DA neuronal cells.

Paragraph 10 The composition of any preceding paragraph wherein the composition further comprises DA neuronal cells and stem cells.

Paragraph 11. The composition of any preceding paragraph wherein the composition further comprises cells for administration.

Paragraph 12. A kit comprising the composition of any preceding paragraph.

Paragraph 13. The composition of any preceding paragraph comprising a pharmaceutically acceptable salt of paragraphs 1-12.

### Targets of Cell Therapy

### Neurodegenerative Disorders

As discussed above, cell therapy can be used to treat a wide variety of disorders. For example, Parkinson's disease is a chronic, progressive neurodegenerative disease or movement disorder that affects up to one million people in the United States. Parkinson's disease affects neurologic function by degrading motor skills of the subject and by causing dementia. The pathology of Parkinson's disease includes reduced formation and action of dopamine, which is produced in the dopaminergic neurons of the brain. Previous research of the causes and possible treatments of Parkinson's disease have been directed towards efforts to compensate for the reduced formation and action of dopamine caused by the disease.

Alzheimer's disease is another neurological disorder that affects numerous individuals around the world. Replacement of diseased neurons through cell therapy may help slow or compensate for the loss of function Alzheimer's patients' experience.

Neurodegenerative disorders include, but are not limited to, (1) trauma, (2) stroke, (3) nonspecific anoxia (i.e., anoxia due to drowning, suffocation, etc.), (4) neurodegenerative diseases such as Alzheimer's disease, Parkinson's disease and other primary and secondary Parkinsonian disorders, and amyotrophic lateral sclerosis (ALS), primary lateral sclerosis (PLS), pseudobulbar palsy and progressive bulbar palsy; and (5) mental retardation syndromes associated with progressive neuronal degeneration (e.g., cerebral palsies). These conditions are well known in the art and can be diagnosed by a treating physician.

### Injury

Apart from degenerative disorders, acute injury to neural tissue may lead to loss of neural function. For example, traumatic brain injury can yield cell damage or death by both primary and secondary mechanisms (discussed further below). Head injury in general, whether from a direct impact to the head or from swelling of the brain due to indirect impact, can also reduce the function of neurons. Additionally, spinal cord injury is one of the most publicly recognized forms of acute injury to neural tissue. Cell therapy in this area of neurological disorder is aimed at restoration (complete or partial) function to organs or limbs that have lost function due to an injury. Even modest clinical improvements have the potential to yield great effects in terms of restoration of the activities an individual can perform as well as their quality of life.

Disclosed is a method for the treatment or enhancement of neurologic function in a patient in need of such treatment which involves delivering a neurologic function enhancement effective amount or a neuroprotective-effective amount of DA neuronal cells to a target area of the patient's brain in combination with treating the patient with an antilipemic agent and/or a CSF-1R antagonist. In some embodiments, the target area of the patient's brain includes an area exhibiting neurodegeneration. In other embodiments, the target area includes portions of the brain not exhibiting neurodegeneration.

In accordance with some embodiments there are provided methods directed toward the enhancement of neurologic function in a subject. The methods include delivering a neurologic enhancing effective amount of an antilipemic agent and/or CSF-1R antagonist to the patient and DA neuronal cells to at least one area of the brain of a subject sufficient to cause an enhancement of neurologic functioning.

In some embodiments, a method of treating a patient having neurologic function affected by Parkinson's disease is provided. The method comprises providing a patient having impaired neurologic function affected by Parkinson's disease. The method further comprises delivering an antilipemic agent and/or CSF-1R antagonist to the patient by oral (or, for example, buccal, systemic, nasal, or injection) administration and delivering DA neuronal cells directly to at least one portion of the brain of the patient sufficient to reduce the severity of symptoms of Parkinson's disease in the patient.

In some embodiments, a method of preventing Parkinson's disease in a patient is provided. The method comprises providing a patient having a predisposition towards contracting Parkinson's disease. The method further comprises delivering DA neuronal cells to at least one portion of the brain of the patient sufficient to reduce a probability of the patient contracting Parkinson's disease. In some embodiments, the patient is treated orally with an antilipemic agent and/or CSF-1R antagonist before, during and/or after the DA neuronal cells are delivered.

### Non-Neural Disorders

Many other diseases that affect various tissues are key targets for cell therapy. In some embodiments, liver damage or cancer is treated with cell therapy in order to replace lost or malfunctioning cells. In some embodiments, diabetic patients are treated with pancreatic progenitor cells (or other stem cells differentiated to pancreatic identity) in order to recapitulate loss of insulin secretion.

Cell therapy also plays a major role in treating cardiac damage. Myocardial infarctions and strokes often result in substantial loss of function in portions of the myocardium. Generally, the myocardium is viewed as comprising a terminally differentiated group of cells with limited capacity for self-renewal. Thus, cell therapy to treat cardiac tissue damage represents great potential progress in helping post-infarction or post-stroke patients regain functionality.

As discussed above, cancers are a particularly interesting area from the perspective of cell therapies, in that the standard therapeutic regime for treating the disease, induces damage to host tissues. In the end, the goal is to kill the cancerous cells, and not so many of the host cells that the patient dies. As such, combating these treatment-induced side effects with cell therapy may increase the probability of survival of cancer patients.

Provided is a method for treating a patient having a disorder with an inflammatory component, by administering to the patient a therapeutically effective amount of DA neuronal cells and at least one antilipemic agent and/or at least one CSF-1R antagonist. Such disorders include, but are not limited to, (1) asthma; (2) autoimmune disorders; (3) allergies; and (4) arthritis. In some embodiments, the disorder with an inflammatory component can include inflammation associated with diseases, such as Alzheimer's disease, Parkinson's disease, ALS, atherosclerosis, diabetes (type 1 or type 2), arthritis, multiple sclerosis, sepsis, septic shock, endotoxemia, multiple organ failure, or organ damage, such as liver damage.

In some embodiments, there is provided a method for treating damage or degeneration in non-neural tissue, for example skeletal muscle, the method comprising delivering an effective amount of an antilipemic agent and/or CSF-1R antagonist to an in vitro culture comprising progenitor cells wherein the progenitor cells are muscle, liver, pancreatic, cardiac, blood, or bone progenitor cells.

As discussed more fully below, the use of an antilipemic agent and/or a CSF-1R antagonist (either by contacting transplanted cells or pre-treating the recipient of transplanted cells) in combination with cell therapy is believed to enhance the efficacy of the cell therapy and leads to a more pronounced therapeutic effect. Moreover, contacting stem, precursor or progenitor cells with an antilipemic agent and/or a CSF-1R antagonist, in some embodiments, is believed to positively impact stem, precursor or progenitor cells in a fashion which makes them more suitable for use in cell therapy. Additionally, use of an antilipemic agent and/or a CSF-1R antagonist (either by contacting transplanted cells or pre-treating the recipient of transplanted cells), in some embodiments, is believed to not only enhance the effects of exogenously administered stem cells used in cell therapy, it is also believed to positively affect endogenous stem cells and/or progenitor cells (e.g., resident neural progenitors or resident cardiac progenitors) such that the combination of administered cell and endogenous cells yields a synergistically enhanced therapeutic effect.

As discussed herein, numerous other diseases are treated with combination of appropriate stem, precursor, or progenitor cells and an antilipemic agent and/or a CSF-1R antagonist.

### Methods

Administering DA neuronal cells in combination with administering an antilipemic agent and/or a CSF-1R antagonist has been identified as capable of promoting survival of neural cells in vitro and in vivo. It is believed that the composition (treating a patient with an antilipemic agent or a CSF-1R antagonist and delivering DA neuronal cells to the patient's target tissue via oral buccal, systemic, nasal, or injection administration) will rescue neurons that would usually shrink, die or disintegrate following traumatic brain damage or as a result of a chronic neurodegenerative disorders. Furthermore, it is expected that the combination therapy has utility as a modulator of inflammation. The combination therapy (treating the patient with DA neuronal cells in combination with administering an antilipemic agent and/or a CSF-1R antagonist) also promotes survival of neural cells.

The combination therapy promotes neuron survival and inhibits aspects of the immune response to cerebral cortex lesions, in particular the appearance and invasion of macrophages and microglia at the site of injury. Accordingly, the combination therapy may be used for treatment of disorders involving acute neural degeneration (stroke and traumatic brain damage), as well as for treatment of several chronic neurodegenerative disorders including Parkinson's disease and Alzheimer's disease. In the latter applications, it is believed that the composition inhibits both neuron death and the brain's immune response to degenerating elements, which should slow the progress of these disorders and attendant decline of behavioral performance. Additionally, combination therapy may be used to treat disorders associated with inflammation.

Several embodiments described herein provide methods directed toward the enhancement of neurologic function in a subject. In some embodiments, the methods include delivering a neurologic enhancing amount of an antilipemic agent and/or a CSF-1R antagonist orally to the patient and delivering DA neuronal cells to at least one area of the brain of a subject.

In some embodiments, the damaged portion of the brain (or other tissue) can comprise the entire brain (or tissue), or portions thereof (e.g., less than 0.1%, 0.5%, 1%, 5%, 10%, 15%, 25%, 50%, or 75% of the target area).

### Treating a Patient

### Preventing Disease

Disclosed is a method for preventing or reducing the severity of neurodegeneration in a subject, includes delivering DA neuronal cells to at least one area of the brain of a subject and administering an antilipemic agent and/or a CSF-1R antagonist to the subject sufficient to prevent or reduce the severity, or reduce the incidence of neurodegeneration in the subject.

In some embodiments, the method treats a subject suffering from Parkinson's disease or other primary and secondary Parkinsonian disorders, including but not limited to idiopathic Parkinson's, vascular parkinsonism, drug-induced parkinsonism and non-ideopathic Parkinson's disease disorders including but not limited to Parkinson's due to mutations in the Parkin gene and other familial and genetic causes of the diseases. The method includes delivering DA neuronal cells to at least one target area of the brain of the subject and administering an antilipemic agent and/or a CSF-1R antagonist to the subject sufficient to prevent, reduce the severity, or reduce the incidence of Parkinson's disease or other primary and secondary Parkinsonian disorders in the subject.

In some embodiments, the target area of the brain may be all of the brain or a specific area of the brain including, but not limited to, an area associated with a particular cognitive or motor function, an area exhibiting neurodegeneration, the cortex, and/or an area that has been affected by trauma. The subject may have a cognitive or motor impairment such as from neurodegeneration or the subject may be healthy, i.e., not have meaningful neurodegeneration.

The target area may be an area of the brain affected by disease or trauma that has been identified such as by using standard medical imaging techniques, it may be a portion of the brain that is known to control certain functions or processes, or it may be any section of the brain, including but not limited to the cortex, cerebellum and other brain regions.

A patient may be treated with an antilipemic agent for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 60 days, 75 days, 100 days, 150 days, 200 days, 365 days prior to administration of the DA neuronal cells. A patient may be treated with fenofibrate for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells.

A stem cell, precursor cell population, progenitor cell population or DA neuronal cell population may be contacted with an antilipemic agent for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells. A stem cell, precursor cell population, progenitor cell population or DA neuronal cell population may be contacted with fenofibrate for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells.

A patient may be treated with an CSF-1R antagonist for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells. A patient may be treated with pexidartinib for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells.

A stem cell, precursor cell population, progenitor cell population or DA neuronal cell population may be contacted with a CSF-1R antagonist for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells. A stem cell, precursor cell population, progenitor cell population or DA neuronal cell population may be contacted with pexidartinib for 1 hour, 2 hours, 3 hours, 4 hours, 5 hours, 6 hours, 7 hours, 8 hours, 9 hours, 10 hours, 1 day, 2 days, 3 days, 4 days, 5 days, 6 days, 7 days, 8 days, 9 days, 10 days, 15 days, 30 days, 45 days, 75 days, 100 days, 150 days, 200 days, 365 days prior to administration of the DA neuronal cells.

In some embodiments, the patient is treated with an antilipemic agent for longer than a CSF-1R antagonist. In some embodiments, the patient is treated with fenofibrate for longer than pexidartinib. In some embodiments, the patient is treated with a CSF-1R antagonist for longer than an antilipemic agent. In some embodiments, the patient is treated with pexidartinib for longer than fenofibrate. In some embodiments, the patient is treated with a CSF-1R antagonist for the same amount of time as an antilipemic agent. In some embodiments, the patient is treated with pexidartinib for the same amount of time as fenofibrate.

In some embodiments, stem, precursor or progenitor cells are exposed to an antilipemic agent for longer than a CSF-1R antagonist. In some embodiments, stem, precursor or progenitor cells are exposed to fenofibrate for longer than pexidartinib. In some embodiments, stem, precursor or progenitor cells are exposed to a CSF-1R antagonist for longer than an antilipemic agent. In some embodiments, stem, precursor or progenitor cells are exposed to pexidartinib for longer than fenofibrate. In some embodiments, stem, precursor or progenitor cells are exposed to a CSF-1R antagonist for the same amount of time as an antilipemic agent. In some embodiments, stem, precursor or progenitor cells are exposed to pexidartinib for the same amount of time as fenofibrate.

In some embodiments, the stem cells are selected from the group comprising hematopoietic stem cells, endothelial stem cells, neural stem cells, bone marrow stem cells including bone marrow mesenchymal stem cells and bone marrow stromal stem cells, and fetal stem cells.

The length of treatment time and frequency of treatment periods with an antilipemic agent and/or CSF-1R antagonist depends on several factors, including the functional recovery of the patient and the results of imaging analysis. In some cases, such as where the disease is degenerative (e.g. Alzheimer's disease) or where treatment is given to a generally healthy patient, the treatment may continue at chosen intervals indefinitely.

During the treatment, the antilipemic agent and/or the CSF-1R antagonist may be continuously provided, or they may be intermittently provided. During the treatment, the antilipemic agent may be continuously provided and the CSF-1R antagonist may be intermittently provided. During the treatment, the CSF-1R antagonist may be continuously provided and the antilipemic agent CSF-1R antagonist may be intermittently provided. During the treatment, the antilipemic agent and the CSF-1R antagonist may be intermittently provided. During the treatment, the antilipemic agent and/or the CSF-1R antagonist may be continuously provided. Herein treatment means preparing the patient for receiving the transplanted cells, during cell transplantation and/or post transplantation.

### Enhancing Graft Formation

Other aspects of the disclosure include methods for enhancing a cell population graft's long term repopulation capability in a recipient. The method comprises the steps of: a) treating the recipient with at least one antilipemic agent and/or at least one CSF-1R antagonist b) administering to the recipient a therapeutically effective amount of at least one population of cells. In some embodiments, the cell population comprises neural cells. In some embodiments, the cell population comprises DA neuronal cells. In some embodiments, the cell population comprises DA neuronal cells and stem cells. In some embodiments, the antilipemic agent and/or CSF-1R antagonist enhances one or more of the viability, engraftment, proliferation, migration, innervation or differentiation of the administered cells.

Treating with an Antilipemic Agent and/or a CSF-1R Antagonist Before, During and after the Administration Step

Disclosed is a method for improving the efficiency of one or more implanted DA neuronal cell populations.

In one embodiment, the method comprises treating the recipient with a therapeutically effective amount of an antilipemic agent and/or a CSF-1R antagonist before administering a population of cells. In one embodiment, the method comprises treating the recipient with a therapeutically effective amount of an antilipemic agent and/or a CSF-1R antagonist during the administration of a population of cells. In one embodiment, the method comprises treating the recipient with a therapeutically effective amount of an antilipemic agent and/or a CSF-1R antagonist after the administration of a population of cells. In one embodiment, the method comprises treating the recipient with a therapeutically effective amount of an antilipemic agent and/or a CSF-1R antagonist before, during, and after the administration of a population of cells. In some embodiments the population of cells comprises DA neuronal cells.

Stated another way, provided is a method for treating a patient having a neurodegenerative disorder by administering to the patient a therapeutically effective amount of DA neuronal cells and a therapeutically effective amount of at least one antilipemic agent and/or at least one CSF-1R antagonist before, during, and/or after administering the DA neuronal cells.

Administration of an Antilipemic Agent and/or a CSF-1R Antagonist

In some embodiments, the therapeutically effective amount of fenofibrate or pexidartinib is from 10 mg to 1000 mg per day. In some embodiments, the therapeutically effective amount of fenofibrate or pexidartinib is from 50 mg to 500 mg per day. In some embodiments, the therapeutically effective amount of fenofibrate or pexidartinib is from 60 mg to 200 mg per day. In some embodiments, the therapeutically effective amount of fenofibrate or pexidartinib is 500 mg per day. In some embodiments, the therapeutically effective amount of fenofibrate or pexidartinib is 200 mg per day. In some embodiments, the therapeutically effective amount of fenofibrate or pexidartinib is 100 mg per day. In some embodiments, the therapeutically effective amount of fenofibrate or pexidartinib is 50 mg per day. In some embodiments, the therapeutically effective amount of fenofibrate is about 10 mg to about 200 mg. In some embodiments, the therapeutically effective amount of fenofibrate is about 10 mg to about 200 mg in the form of an immediate release tablet. In some embodiments, the therapeutically effective amount of fenofibrate is about 10 mg to about 200 mg fenofibrate in the form of a single matrix tablet. In some embodiments, fenofibrate or pexidartinib is administered in a sub-therapeutic dose. In some embodiments, fenofibrate or pexidartinib is administered in a therapeutic dose.

In some embodiments, the therapeutically effective amount of fenofibrate is from 10 mg to 1000 mg per day and the therapeutically effective amount of pexidartinib is from 10 mg to 1000 mg per day. In some embodiments, the therapeutically effective amount of fenofibrate is from 10 mg to 1000 mg per day and the therapeutically effective amount of pexidartinib is from 10 mg to 50 mg per day. In some embodiments, the therapeutically effective amount of fenofibrate is from 10 mg to 1000 mg per day and the therapeutically effective amount of pexidartinib is from 500 mg to 1000 mg per day. In some embodiments, the therapeutically effective amount of fenofibrate is from 10 mg to 50 mg per day and the therapeutically effective amount of pexidartinib is from 10 mg to 1000 mg per day. In some embodiments, the therapeutically effective amount of fenofibrate is from 500 mg to 1000 mg per day and the therapeutically effective amount of pexidartinib is from 10 mg to 1000 mg per day.

In some embodiments, the antilipemic agent and/or CSF-1R antagonist is administered both in vitro and in vivo. When it is administered in vitro it is contacted with the cells to be transplanted. When it is administered in vivo it is contacted with the transplanted cells, endogenous cells or both.

In some embodiments, an ongoing regime of antilipemic agent and/or CSF-1R antagonist administration is used (e.g., daily, twice daily administration, either in vitro, in vivo, or both). Many varied patterns of antilipemic agent and/or CSF-1R antagonist administration can be used, based on the specific disease or injury, and cell type being used for cell therapy.

In some embodiments, the antilipemic agent and/or CSF-1R antagonist is delivered continuously. In some embodiments, the antilipemic agent and/or CSF-1R antagonist is delivered intermittently, i.e., it is administered for a first period of time, stopped, and then administered for a second period of time. In some embodiments, the antilipemic agent is administered continuously and the CSF-1R antagonist is delivered intermittently. In some embodiments, the CSF-1R antagonist is administered continuously and the antilipemic agent is delivered intermittently.

Aspects and embodiments of the methods disclosed herein can be further understood by in the following numbered paragraphs:

Paragraph 1. A method of enhancing cell therapy comprising administering to the patient a therapeutically effective amount of fenofibrate.

Paragraph 2. A method of enhancing cell therapy comprising administering to the patient a therapeutically effective amount of at least one antilipemic agent.

Paragraph 3. A method of enhancing cell therapy comprising administering to the patient a therapeutically effective amount of pexidartinib.

Paragraph 4. A method of enhancing cell therapy comprising administering to the patient a therapeutically effective amount of at least one CSF-1R antagonist.

Paragraph 5. The method of any preceding paragraph wherein the administering occurs pitot to cell therapy, during cell therapy, after cell therapy and combinations thereof.

Paragraph 6. A method of enhancing cell therapy comprising administering to the patient a therapeutically effective amount of fenofibrate and/or pexidartinib and not clorofibrate, benzafibrate and/or fenofibric acid.

### Site of Implantation

The site of implantation is chosen to permit the implanted cells to regenerate the damaged tissue, for example by directly repopulating the damaged or degenerating tissue, or by supporting the growth or proliferation of endogenous cells.

In some embodiments, the target area (site of implantation) of the brain may be all of the brain or a specific area of the brain including, but not limited to, an area associated with a particular cognitive or motor function, an area exhibiting neurodegeneration, the cortex, and/or an area that has been affected by trauma. The subject may have a cognitive or motor impairment such as from neurodegeneration or the subject may be normal.

### Delivery of Cells

The stem cells may be delivered by numerous routes, including direct injection, catheter-based approaches, intravascular administration, stereotactic-guided delivery, etc.

In some embodiments, graft enhancement caused by treating the patient with an antilipemic agent or CSF-1R antagonist is manifest as an increased viability of the transplanted cells. In some embodiments, the increased viability is advantageous because the cells are present in a tissue that is afflicted with a disease, and therefore may present one or more cellular pro-death signals. In some embodiments, an antilipemic agent and/or a CSF-1R antagonist enhances viability of transplanted cells by increasing the resistance of the cells to apoptotic factors. In some embodiments, anti-apoptotic pathways are upregulated in transplanted cells. While not the only factor to consider, enhanced viability of cells, in some embodiments, is of particular importance, as replacement of damaged or diseased cells may be the most efficacious when the replacement cell is likely to survive.

In some embodiments, the increase in proliferation results in a substantially larger population of stem cells that can functionally replace (partially or fully) the damaged or diseased cells of the host. For example, administration of a small population of neural progenitor cells to an individual with Parkinson's disease (e.g. by stereotactic delivery of the cells to a target region of the brain) and co-administration of an antilipemic agent and/or a CSF-1R antagonist, in some embodiments, induces proliferation of the neural progenitor cells to a degree which compensates for the loss of speech or motor control associated with Parkinson's. In some embodiments, treatment of the patient with an antilipemic agent and/or a CSF-1R antagonist can enhance the proliferation of endogenous stem cells to potentiate the effects of the exogenously delivered cells. In some embodiments, the antilipemic agent and/or a CSF-1R antagonist dosage are tailored to generate a desired proliferative growth curve (e.g., decreasing frequency and/or intensity of an antilipemic agent and/or a CSF-1R antagonist over time to reduce the proliferative stimulation). In this manner, uncontrolled proliferation of either endogenous or administered cells (or other cells receiving an antilipemic agent and/or a CSF-1R antagonist) is avoided.

In some embodiments, treating the patient with an antilipemic agent and/or a CSF-1R antagonist improves the migration of transplanted cells. In some embodiments, chemoattractant signals present in the target tissue induce the migration of cells (endogenous or exogenous) to a desired location. In some embodiments, an antilipemic agent and/or a CSF-1R antagonist potentiates the response of the transplanted cells to such a signal, thereby allowing a more rapid repositioning of cells to a desired location. Once in its desired location, in some embodiments, the additional effects of an antilipemic agent and/or a CSF-1R antagonist described herein allow the cell to more rapidly or efficiently provide a therapeutic benefit (e.g., provide function that is lost due to damage or disease). In some embodiments, chemorepellant signals drive transplanted cells away from an undesired location. In some embodiments, the combination of chemoattractant and chemorepellant signals work in concert to direct the transplanted cells to a desired location. In some embodiments, the chemoattractant and/or chemorepellant signals are exogenously administered as well. In some such embodiments, a series of injections of a chemoattractant compound are pre-delivered to a target tissue in order to generate a gradient of signal for the administered cells to respond to. Post-administration, the cells migrate along this gradient, thereby coming to rest at a desirable position.

In some embodiments, engraftment of the transplanted cells is improved by administration of an antilipemic agent and/or a CSF-1R antagonist to the patient. As discussed above this may be particularly advantageous in certain therapeutic applications, such as for example cell therapy directed to target tissues that experience shear flow, flex, or other forces that may dislodge the cells. For example, in some embodiments, an antilipemic agent and/or a CSF-1R antagonist and DA neuronal cells are administered, and engraftment is enhanced (as compared to progenitor cells alone). This is particularly advantageous because the blood flow through the brain could wash the administer cells out of the target organ. Moreover, the constant flex of the microglia may dislodge the administered cells. As such, the increased engraftment of administered cells increases the efficacy of the therapy due to the retention of a larger number of cells at the target site.

### Function of Cells

In some embodiments, the function of stem cells is improved by the administration of an antilipemic agent and/or a CSF-1R antagonist to the patient. In some embodiments, the function of administered progenitor cells (or endogenous stem cells) is improved by treating the patient with an antilipemic agent and/or a CSF-1R antagonist (antecedent, concurrent, and post-engraftment). By way of example, an antilipemic agent and/or a CSF-1R antagonist may promote increase firing of a neuron (derived from a neural progenitor). Similarly, in some embodiments, increased neurotransmitter release results. In some embodiments, alterations in cell biology occur (e.g., increased or decreased axonal transport) which are beneficial to the function of the neuron.

In some embodiments, an antilipemic agent and/or a CSF-1R antagonist positively impacts the administered cells which are themselves enhanced in one or more of the manners described herein. In some embodiments, the effects of the antilipemic agent and/or a CSF-1R antagonist on the transplanted cells results in a cascade that yields beneficial effects to the cells of the damaged or diseased host tissue. For example, in some embodiments, patient treatment with an antilipemic agent and/or a CSF-1R antagonist (antecedent, concurrent, and post-engraftment) induces pro-survival paracrine factor (e.g., growth factors, immunosuppressive molecules) release from the progenitor cells, which, in turn, improves the survival of the damaged or diseased host tissue. Thus, in some embodiments, the characteristics of the progenitor cells are enhanced, which improves cell therapy. In some embodiments, the antilipemic agent and/or a CSF-1R antagonist-treated progenitor cells become a source of a signal that improves damaged or diseased host tissue (e.g., the cells are a vehicle for a beneficial effect rather than providing the effect directly).

In some embodiments, the progenitor cells are responsive to the in vivo environment into which they are transplanted. For example, tissue damage or disease is often associated with various signaling cascades, which, in balance, determine the outcome of a subset of cells (or the entire tissue). In some embodiments, the administered cells detect, and subsequently respond to the milieu of damage, disease, and/or inflammatory signals in the target tissue. In some embodiments, (as discussed above), certain characteristics of the administered cells advantageously alter the balance, to the benefit of the survival of the administered cells and/or the cells of the host tissue because they have been contracted with an antilipemic agent and/or CSF-1R antagonist prior to or after delivery. For example, MSCs, respond to the pro-inflammatory environment in a damaged tissue by releasing anti-inflammatory cytokines, altering T-cell function, and/or altering monocyte maturation. Thus, MSCs may be of particular benefit in allogeneic transplants. Also, in some embodiments, other progenitor cell types possess similar environmentally-responsive characteristics. Such cells, with the ability to respond to local signals, generate counteractive local and/or paracrine signals, and effectively alter the local environment in a beneficial (e.g., pro-survival or regeneration of function manner) are used in some embodiments. In some embodiments, a combination of these mechanisms results. As discussed, such cells are particularly advantageous in allogeneic transplants, though in some embodiments, they are used in autologous cell transplants. The pluripotent cells used herein may be of autologous, syngeneic or allogeneic related (matched siblings or haploidentical family members) or unrelated fully mismatched source.

In some embodiments, a method is provided for treating damage or illness in the central nervous system in a mammal or human, comprising delivering an effective amount of an antilipemic agent and/or a CSF-1R antagonist to an in vitro culture comprising stem cells, (e.g. stem cells, induced pluripotent cells, genetically modified adult cells, adult cells etc.) and implanting the cells into the central nervous system of a mammal or human.

In some embodiments, treatment of a patient comprises implantation of progenitor cells into the central nervous system (“CNS”) of the patient. Following implantation, the progenitor cells differentiate to form one or more cell types of the central nervous system. The implanted cells may serve any of a variety of purposes, including replacement of cells or tissues that have been irreparably damaged, repair of a portion of the CNS, enhance the production of important CNS neurochemicals such as dopamine, seratonin, endogenous opioid peptides, and the like. Implantation of progenitor cells may be performed alone, or it may be done in combination with the methods of enhancing neurologic functioning, as described herein. For example, the progenitor cells may be treated with an therapeutic agent or combination of therapeutic agents in addition to the antilipemic agent and/or a CSF-1R antagonist, prior to, during, after or combinations thereof implantation. By way of example, the additional agent may be selected from the group consisting of pharmaceutical compounds, cytokines, growth factors, neurotransmitters, hormones, trophic factors, transcription factors, monoclonal antibodies, polyclonal antibodies, or signal transduction molecules. In some embodiments, the agent or combination of agents may have the effect of stimulating or mobilizing progenitor cells.

Procedures for Antilipemic Agent and/or a CSF-1R Antagonist Treatment

In certain embodiments, a patient is treated by identifying a plurality of treatment sites (e.g., at least about 10) in the patient's brain or other target tissue, administering at least one plurality of cells (stem, precursor, progenitor, DA neuronal cells or combinations thereof) to each of the treatment sites, and administering the patient with an antilipemic agent and/or a CSF-1R antagonist (before, during, after or combinations thereof of administering the cells). In some embodiments, the cells are treated with an antilipemic agent and/or a CSF-1R antagonist prior to administration to a patient. In some embodiments, the cells are treated with an antilipemic agent and/or a CSF-1R antagonist prior to implantation as well as one or more times post-implantation.

In some embodiments, a single treatment site is treated with the cells. In some embodiments, the treatment site is selected from the group consisting of, heart, lungs, liver, pancreas, kidney, spleen, intestine, bone, bone marrow, teeth/gums, skeletal or smooth muscle, skin, or combinations thereof. Each of the treatment sites can be treated with the stem, precursor, progenitor, or DA neuronal cells.

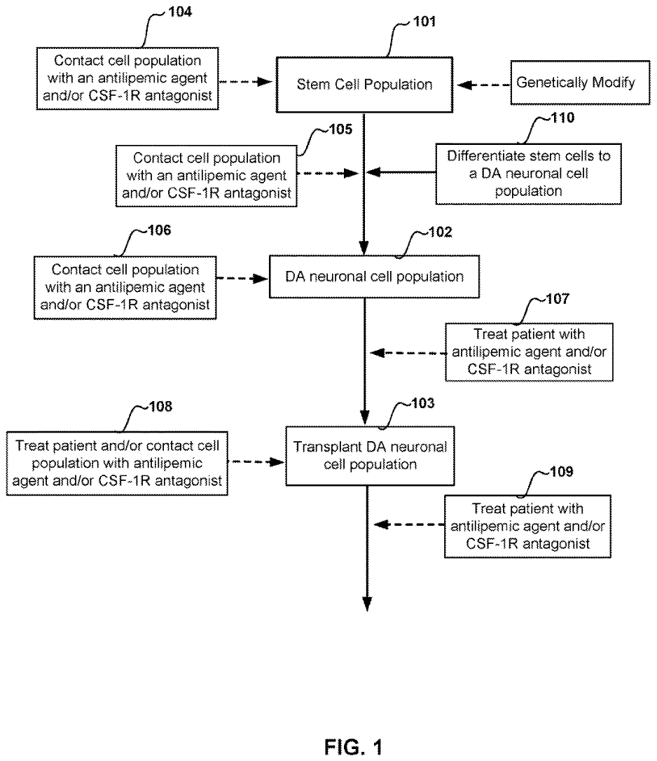

FIG. 1 is a flow diagram of an example method disclosed herein. As shown in FIG. 1, the general method is to differentiate 110 stem cells 101 to a progenitor cell population 102 and transplant the differentiated cell population 103. At each step, there are optional steps as indicted by a dashed line. The stem cells can be contacted with an antilipemic agent and/or a CSF-1R antagonist 104, or the differentiating cells can be contacted with an antilipemic agent and/or a CSF-1R antagonist 105 or the DA neuronal cells can be contacted with an antilipemic agent and/or a CSF-1R antagonist 106, or the transplanted DA neuronal cells can be contacted with an antilipemic agent and/or a CSF-1R antagonist 108, or combinations thereof. Cell administration occurs prior to treating the patient with an antilipemic agent and/or a CSF-1R antagonist (e.g. before 107), while treating the patient with an antilipemic agent and/or a CSF-1R antagonist (e.g. before 108), after treating the patient with an antilipemic agent and/or a CSF-1R antagonist (e.g. before 109) or combinations thereof. In some embodiments, treating a patient with an antilipemic agent and/or a CSF-1R antagonist and/or cell delivery occurs multiple times over a therapeutic regime. As discussed above, though not shown in FIG. 1, similar methods are used to treatment of other tissues, in some embodiments.

In some embodiments, the treatment may be terminated after one treatment period, while in other embodiments, the treatment may be repeated for at least two treatment periods, at least five treatment periods at least ten treatment periods, at least fifty treatment periods, at least one-hundred treatment periods. In some embodiments, the treatment may be terminated after one treatment period, while in other embodiments, the treatment may be repeated for at least ten treatment periods. The time between subsequent treatment periods is preferably at least about five minutes, at least about 1 to 2 days, at least about one week, at least about two weeks, at least about one month, at least about two months, at least about three months, at least about six months, at least about one year. The length of treatment time and frequency of treatment periods can depend on several factors, including the functional recovery of the patient and the results of imaging analysis of the patient. In certain embodiments, one or more treatment parameters can be adjusted in response to a feedback signal from a device (e.g., magnetic resonance imaging) monitoring the patient. Herein treatment means the combination therapy.

In addition to the combination of cell therapy and treating a patent with an antilipemic agent and/or a CSF-1R antagonist, in certain embodiments, the combination is combined with other types of treatments for an improved therapeutic effect. Treatment can comprise administering the cells to a target area of the brain concurrently with applying an electromagnetic field to the brain. Similar approaches are taken to treat other target tissues. In such embodiments, the electromagnetic field has an efficacious field strength as described in U.S. Pat. No. 6,042,531 issued to Holcomb, which is incorporated in its entirety by reference herein. In certain embodiments, the electromagnetic field comprises a magnetic field, while in other embodiments, the electromagnetic field comprises a radio-frequency (RF) field. As another example, treatment can comprise administering the DA cells to the patient to a target area of the brain concurrently with applying an efficacious amount of ultrasonic energy to the brain. Such a system can include systems for ultrasonic treatment, e.g., as described in U.S. Pat. No. 5,054,470 issued to Fry et al., which is incorporated in its entirety by reference herein.

Assessing Efficacy of an Antilipemic Agent and/or a CSF-1R Antagonist and Cell Therapy

Depending on the disease or injury treated by administering an antilipemic agent and/or a CSF-1R antagonist to a patient in combination with cell therapy, various endpoints are used to assess the efficacy of the therapies. For example, neurologic function scales can be used to quantify or otherwise characterize the efficacy of various embodiments described herein. Neurologic function scales generally use a number of levels or points, each point corresponding to an aspect of the patient's condition. The number of points for a patient can be used to quantify the patient's condition, and improvements in the patient's condition can be expressed by changes of the number of points. One example neurologic function scale used as a clinical tool for diagnosis and determining severity of Parkinson's disease is the Unified Parkinson's Disease Rating Scale (UPDRS) which comprises various sections evaluated by interview and clinical observation. In certain embodiments, two or more of the neurologic function scales can be used in combination with one another, and can provide longer-term measurements of efficacy (e.g., at three months).

In certain embodiments described herein, a patient exhibiting symptoms of Parkinson's disease is treated by administering an antilipemic agent and/or a CSF-1R antagonist to the patient and administering DA neuronal cells to the patient's brain which is expected to produce at least a 2% average difference between the treated group and a placebo group on at least one neurologic function scale (e.g., UPDRS) analyzed in dichotomized or any other fashion. Certain other embodiments produce at least a 4% average difference, at least a 6% average difference, or at least a 10% average difference between treated and placebo groups on at least one neurologic function scale analyzed in dichotomized or any other fashion. In certain embodiments, the treatment by administering an antilipemic agent and/or a CSF-1R antagonist to the patient and administering DA neuronal cells to the patient's brain produces a change in the patient's condition. In certain such embodiments, the change in the patient's condition corresponds to a change in the number of points indicative of the patient's condition. In certain such embodiments, the treatment produces a change of one point, a change of two points, a change of three points, or a change of more than three points on a neurologic function scale.

Aspects and embodiments of the methods disclosed herein can be further understood by in the following numbered paragraphs:

Paragraph 1. A method for enhancing the suitability of cells for use in neural cell therapy comprising:
delivering an antilipemic agent and/or a CSF-1R antagonist to a mammal; and
administering cells to the mammal.

Paragraph 2. The method of any preceding paragraph, wherein at cells are DA neuronal cells.

Paragraph 3. The method of any preceding paragraph, wherein the cells are derived from the group of stem cell sources comprising adult stem cells, embryonic stem cells, placenta-derived stem cells, bone marrow-derived stem cells, mesenchymal stem cells, adipose stem cells, and induced pluripotent stem cells.

Paragraph 4. The method of any preceding paragraph, wherein the cells are for use in cell therapy to treat a neurological disease or injury.

Paragraph 5. The method of paragraph 4, wherein the neurological disease or injury is selected from the group consisting of Parkinson's disease, Alzheimer's disease, Huntington's disease, dopaminergic impairment, depression, stroke, head trauma, neurodegeneration, and dementia.

Paragraph 6. The method of any preceding paragraph, delivering an antilipemic agent and/or a CSF-1R antagonist to a mammal occurs before, during and after the administering step.

Paragraph 7. The method of any preceding paragraph, wherein the cells are treated with an antilipemic agent and/or a CSF-1R antagonist prior to the administering step.

Paragraph 8. The method of paragraph 3, wherein the stem cells are treated with an antilipemic agent and/or a CSF-1R antagonist prior to differentiating the stem cells.

Paragraph 9. The method of any preceding paragraph, wherein the cells are administered to impaired tissue.

Paragraph 10. The method of any preceding paragraph, wherein the cells are administered to healthy tissue.

Paragraph 11. The method of any preceding paragraph, wherein the cells are stem, precursor, or progenitor cells.

Paragraph 12. The method of any preceding paragraph, wherein the cells are neuron, brain, heart, liver, bone or muscle progenitor cells.

Paragraph 13. The method of any preceding paragraph, delivering an antilipemic agent and/or a CSF-1R antagonist to a mammal occurs before the administering step.

Paragraph 14. The method of any preceding paragraph, delivering an antilipemic agent and/or a CSF-1R antagonist to a mammal occurs during the administering step.

Paragraph 15. The method of any preceding paragraph, delivering an antilipemic agent and/or a CSF-1R antagonist to a mammal occurs after the administering step.

Paragraph 16. The method of any preceding paragraph, wherein the cells comprise stem, precursor, and progenitor cells.

Paragraph 17. The method of any preceding paragraph, wherein the cells comprise stem, and progenitor cells.

Paragraph 18. The method of any preceding paragraph, wherein the cells comprise stem, and DA neuronal cells.

Paragraph 19. The method of any preceding paragraph, wherein the cells are treated with an antilipemic agent and/or a CSF-1R antagonist after the administering step.

Paragraph 20. The method of any preceding paragraph, wherein the cells are treated with an antilipemic agent for a first time period and a CSF-1R antagonist for a second time period wherein the first time period is shorter than the second time period.

Paragraph 21. The method of any preceding paragraph, wherein the cells are treated with an antilipemic agent for a first time period and a CSF-1R antagonist for a second time period wherein the first time period is longer than the second time period.

Paragraph 22. The method of any preceding paragraph, wherein the cells are not treated with clorofibrate, benzafibrate and/or fenofibric acid.

### Method of Differentiating Cells

In some embodiments, treatment of a patient suffering from Parkinson's disease and other primary and secondary Parkinsonian disorders comprises implantation of progenitor cells into the brain of the patient. As discussed, herein, the patient may be pre-treated with an antilipemic agent and/or a CSF-1R antagonist, or may be treated post-implantation, or treated during transplantation, or combinations thereof. Following implantation, the progenitor cells differentiate to form one or more brain cell types. In some embodiments, the cells functionally replace the damaged brain cells of the patient, thereby restoring brain function (partially or fully). In some embodiments, progenitor cells are inoculated and grown in a cell culture in vitro, while being exposed to an antilipemic agent and/or a CSF-1R antagonist before, during after, or combinations thereof when differentiating from stem cells.

Because, in some embodiments, the DA neuronal cells and an antilipemic agent and/or a CSF-1R antagonist is applied directly to the cell culture in vitro and does not travel through intervening body tissue, the concentration of the antilipemic agent and/or a CSF-1R antagonist selected to be delivered to the cell is generally less than what is used for patient treatment. In some embodiments, the treated cells are implanted following treatment. In other embodiments, at least some treated cells remain in culture to maintain the cell line for later use. In some embodiments, the concentration of the antilipemic agent for the in vitro culture is from about 0.2 μM to about 4 μM, from about 0.2 μM to about 8 μM, from about 0.2 μM to about 16 μM about, from about 0.2 μM to about 50 μM, from about 0.2 μM to about 100 μM or any range derivable therein. In some embodiments, the concentration of the CSF-1R antagonist agent for the in vitro culture is from about 0.2 μM to about 4 μM, from about 0.2 μM to about 8 μM, from about 0.2 μM to about 16 μM about, from about 0.2 μM to about 50 μM, from about 0.2 μM to about 100 μM or any range derivable therein. In some embodiments, the concentration of fenofibrate for the in vitro culture is from about 0.2 μM to about 4 μM, from about 0.2 μM to about 8 μM, from about 0.2 μM to about 16 μM about, from about 0.2 μM to about 50 μM, from about 0.2 μM to about 100 μM or any range derivable therein. In some embodiments, the concentration of pexidartinib agent for the in vitro culture is from about 0.2 μM to about 4 μM, from about 0.2 μM to about 8 μM, from about 0.2 μM to about 16 μM about, from about 0.2 μM to about 50 μM, from about 0.2 μM to about 100 μM or any range derivable therein. In some embodiments, the concentration of pexidartinib for the in vitro culture is less than the concentration of fenofibrate. In some embodiments, the concentration of fenofibrate for the in vitro culture is less than the concentration of pexidartinib. In some embodiments, the concentration of fenofibrate and pexidartinib is the same. In some embodiments, the exposure of cells (stem, precursor or progenitor) to pexidartinib is for a longer time period than for fenofibrate. In some embodiments, the exposure of cells (stem, precursor or progenitor) to fenofibrate is for a longer time period than for pexidartinib. In some embodiments, the exposure of cells (stem, precursor or progenitor) to fenofibrate is for the same amount of time as for pexidartinib.

After in vitro treatment of cells using an antilipemic agent and/or a CSF-1R antagonist, the cells are transplanted or implanted to a recipient site in a patient. In some embodiments, the treatment prior to transplantation or implantation includes culturing cells sufficient for implantation. The recipient site may be a site of injury, illness, or defect, or it may be a region of relatively healthy tissue. In some embodiments, the recipient site and/or the region surrounding such site is treated with DA neuronal cells and an antilipemic agent and/or a CSF-1R antagonist according to the methods described supra, before and/or after implantation to enhance the rate at which the implanted cells are integrated with surrounding tissue at the recipient site.

Disclosed is a method for improving the efficiency of one or more cell populations by contacting the cells with an antilipemic agent and/or CSF-1R antagonist.

Disclosed is a method for improving the efficiency of one or more DA neuronal cell populations for implantation by contacting the DA neuronal cells with an antilipemic agent and/or CSF-1R antagonist. In some embodiments, the DA neuronal cells are derived from one of a variety of stem cell sources consisting of adult stem cells, embryonic stem cells, placenta-derived stem cells, bone marrow-derived stem cells, mesenchymal stem cells, adipose stem cells, and induced pluripotent stem cells. In some embodiments, the DA neuronal cells are derived from neural stem cells. In other embodiments, the DA neuronal cells differentiate in vivo after administration to a cell therapy subject. In some embodiments, in vitro differentiation is not complete (e.g., the cells are not terminally differentiated), but are lineage committed.

In some embodiments, the administered cells are autologous with respect to the recipient. In other embodiments, the administered cells are allogeneic with respect to the recipient. In some embodiments, the administered DA neuronal cells are autologous with respect to the recipient. In other embodiments, the administered DA neuronal cells are allogeneic with respect to the recipient. In one embodiment, mesenchymal stem cells are used in allogeneic transplants due to the ability of the cells to modulate the immune response in the target tissue. In one embodiment, the DA neuronal cells have altered T-cell or antigen presenting cell function, thereby reducing immunologic rejection of transplanted cells. In one embodiment, the DA neuronal cells additionally reduce fibrosis in the target tissue.

In some embodiments, the DA neuronal cells are for use in cell therapy to treat a neurological disease or injury. For example, in some embodiments, the tissue with impaired function is neural tissue having impaired function due to degenerative neural disease. In some embodiments, the DA neuronal cells are administered to a subject for the treatment of Parkinson's disease or other primary and secondary Parkinsonian disorders. In some embodiments, other degenerative diseases, such as dopaminergic impairment, Alzheimer's, amyotrophic lateral sclerosis, Huntington's disease, and/or dementia are treated. In some embodiments, impaired neural function is a result of injury to the neurons.

In one embodiment, the DA neuronal cells are used to treat the damage due to stroke. In one embodiment, cerebral ischemia (including focal cerebral ischemia), traumatic brain injury, and/or physical trauma such as crush or compression injury in the CNS, including a crush or compression injury of the brain, spinal cord, nerves or retina, is treated.

Modified or unmodified pluripotent cell can be differentiated to a midbrain DA neuron as is known in the art. In some embodiments, the midbrain DA neuron cell population differentiated from a modified pluripotent cell or unmodified pluripotent cell comprises three cell populations: A9 dopamine neurons, astrocytes and vascular leptomeningeal cells (VLMC). In some embodiments, the pluripotent cells are differentiated using a mono-SMAD, or dual-SMAD method disclosed herein. In some embodiments, the pluripotent cells are differentiated using a mono-SMAD, dual-SMAD or other approach. In some embodiments, after the unmodified pluripotent cell are differentiated to unmodified midbrain DA neurons, the unmodified midbrain DA neurons are incubated with an antilipemic agent and/or CSF-1R antagonist. In some embodiments, after the modified pluripotent cell are differentiated to modified midbrain DA neurons, the modified midbrain DA neurons are incubated with an antilipemic agent and/or CSF-1R antagonist.

Treating Stem Cells with an Antilipemic Agent and/or CSF-1R Antagonist

In one embodiment, the method further comprises the step of applying the antilipemic agent and/or a CSF-1R antagonist to the stem cells prior to differentiation.

In some embodiments, there is provided a method for treating damage or illness in the central nervous system in a mammal or human, comprising delivering an effective amount of an antilipemic agent and/or CSF-1R antagonist to an in vitro culture comprising stem cells, differentiating the treated stem cells to DA neuronal cells and implanting the DA neuronal cells into the central nervous system of a mammal or human.

Treating Differentiating Cells with an Antilipemic Agent and/or CSF-1R Antagonist

In some embodiments, there is provided a method for treating damage or illness in the central nervous system in a mammal or human, comprising delivering an effective amount of an antilipemic agent and/or CSF-1R antagonist to an in vitro culture comprising stem cells, differentiating the treated stem cells to DA neuronal cells while providing an effective amount of an antilipemic agent and/or CSF-1R antagonist to the differentiating cells and implanting the DA neuronal cells into the central nervous system of a mammal or human.

Treating Progenitor Cells with an Antilipemic Agent and/or CSF-1R Antagonist

In some embodiments, there is provided a method for treating damage or illness in the central nervous system in a mammal or human, comprising delivering an effective amount of an antilipemic agent and/or CSF-1R antagonist to an in vitro culture comprising progenitor cells, and implanting the treated cells into the central nervous system of a mammal or human.

Method of Treating a Patient with Progenitor Cells Contacted with an Antilipemic Agent and/or CSF-1R Antagonist

In some embodiments, a method of treating the central nervous system of a patient is provided. The method comprises identifying a patient exhibiting symptoms of damage to the central nervous system. The method further comprises contacting an in vitro culture comprising cells with an antilipemic agent and/or CSF-1R antagonist. The method further comprises implanting the treated cells into the central nervous system of the patient. In some embodiments, the cells are stem cells. In some embodiments, the cells are progenitor cells. In some embodiments, the cells are DA neuronal cells.

In some embodiments, the antilipemic agent and/or CSF-1R antagonist is delivered to the DA neuronal cells in vitro, while in some embodiments, the antilipemic agent and/or CSF-1R antagonist is delivered to the DA neuronal cells in vivo (e.g., post administration of the cells to the subject).

In some embodiments, the antilipemic agent and/or CSF-1R antagonist is delivered to the patient orally and the DA neuronal cells in vitro. In some embodiments, the antilipemic agent and/or CSF-1R antagonist is delivered to the patient orally and the DA neuronal cells in vitro and in vivo.

### Method of Treatment

Provided is a method of treating a brain disease or disorder in a subject in need thereof, the method comprising administering to the subject a therapeutically effective amount of the isolated population of cells comprising DA neural cells. Provided is a method of treating a brain disease or disorder in a subject in need thereof, the method comprising administering to the subject a therapeutically effective amount of the isolated population of cells comprising DA neural cells in combination with an antilipemic agent and/or a CSF-1R antagonist.

Provided is a method of treating a brain disease or disorder in a subject in need thereof, the method comprising administering to the subject a therapeutically effective amount of the isolated population of cells comprising DA neural cells in combination with an antilipemic agent or a CSF-1R antagonist. Provided is a method of treating a brain disease or disorder in a subject in need thereof, the method comprising administering to the subject a therapeutically effective amount of the isolated population of cells comprising DA neural cells in combination with treating the subject with an antilipemic agent. Provided is a method of treating a brain disease or disorder in a subject in need thereof, the method comprising administering to the subject a therapeutically effective amount of the isolated population of cells comprising DA neural cells in combination with treating the subject with fenofibrate. Provided is a method of treating a brain disease or disorder in a subject in need thereof, the method comprising administering to the subject a therapeutically effective amount of the isolated population of cells comprising DA neural cells in combination with treating the subject with fenofibrate. Provided is a method of treating a brain disease or disorder in a subject in need thereof, the method comprising administering to the subject a therapeutically effective amount of the isolated population of cells comprising DA neural cells in combination with treating the subject with a CSF-1R antagonist. Provided is a method of treating a brain disease or disorder in a subject in need thereof, the method comprising administering to the subject a therapeutically effective amount of the isolated population of cells comprising DA neural cells in combination with treating the subject with pexidartinib.

Provided is a method of treating a brain disease or disorder in a subject in need thereof, the method comprising administering to the subject a therapeutically effective amount of the isolated population of cells comprising DA neural cells in combination with treating the subject with antilipemic agent and a CSF-1R antagonist. Provided is a method of treating a brain disease or disorder in a subject in need thereof, the method comprising administering to the subject a therapeutically effective amount of the isolated population of cells comprising DA neural cells in combination with treating the subject with fenofibrate and a CSF-1R antagonist. Provided is a method of treating a brain disease or disorder in a subject in need thereof, the method comprising administering to the subject a therapeutically effective amount of the isolated population of cells comprising DA neural cells in combination with treating the subject with fenofibrate and a CSF-1R antagonist. Provided is a method of treating a brain disease or disorder in a subject in need thereof, the method comprising administering to the subject a therapeutically effective amount of the isolated population of cells comprising DA neural cells in combination with treating the subject with fenofibrate and pexidartinib. Provided is a method of treating a brain disease or disorder in a subject in need thereof, the method comprising administering to the subject a therapeutically effective amount of the isolated population of cells comprising DA neural cells in combination with treating the subject with fenofibrate and pexidartinib.

In one embodiment, provided is a method of engrafting cells in vivo for therapeutic treatment, comprising, a) providing: i) a population of midbrain dopamine (DA) neurons; ii) an antilipemic agent or a CSF-1R antagonist and iii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons; ii) and antilipemic agent and a CSF-1R antagonist and iii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons; ii) and fenofibrate and pexidartinib and iii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, said neurological symptoms are selected from the group consisting of tremor, bradykinesia (extreme slowness of movement), flexed posture, postural instability, and rigidity. In one embodiment, said subject shows reduction of said neurological symptom. In one embodiment, said population of midbrain dopamine (DA) neurons are derived from a cell population selected from the group comprising primates and humans. In one embodiment, said human cells are cells from a patient with a symptom of Parkinson's disease (PD).

Treating with Modified DA Neural Cells

Provided is a method of treating a brain disease or disorder in a subject in need thereof, the method comprising administering to the subject a therapeutically effective amount of the isolated population of cells comprising genetically modified DA neural cells. Provided is a method of treating a brain disease or disorder in a subject in need thereof, the method comprising administering to the subject a therapeutically effective amount of the isolated population of cells comprising genetically modified DA neural cells in combination with an antilipemic agent and a CSF-1R antagonist.

Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with an antilipemic agent or a CSF-1R antagonist. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with an antilipemic agent. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with fenofibrate. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with fenofibrate. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with a CSF-1R antagonist. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with pexidartinib.

Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with antilipemic agent and a CSF-1R antagonist. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with fenofibrate and a CSF-1R antagonist. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with fenofibrate and a CSF-1R antagonist. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with fenofibrate and pexidartinib. Provided is a method of treating a brain disease or disorder in a subject in need thereof, the method comprising administering to the subject a therapeutically effective amount of the isolated population of cells comprising DA neural cells which are miR-155-3p biased or miR-155-5p biased in combination with fenofibrate and pexidartinib.

In some embodiments, the miR-155-3p biased or miR-155-5p biased isolated population of cells are A9 dopamine neurons. In some embodiments, the miR-155-3p biased or miR-155-5p biased isolated population of cells are astrocytes. In some embodiments, the miR-155-3p biased or miR-155-5p biased isolated population of cells are VLMC.

In one embodiment, provided is a method of engrafting cells in vivo for therapeutic treatment, comprising, a) providing: i) a population of midbrain dopamine (DA) neurons wherein miR-155-5p is expressed more than miR-155-3p; ii) antilipemic agent or a CSF-1R antagonist, and ii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons wherein miR-155-5p is expressed more than miR-155-3p; ii) antilipemic agent and a CSF-1R antagonist, and ii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons wherein miR-155-5p is expressed more than miR-155-3p; ii) fenofibrate and pexidartinib, and ii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function.

In one embodiment, provided is a method of engrafting cells in vivo for therapeutic treatment, comprising, a) providing: i) a population of midbrain dopamine (DA) neurons wherein miR-155-3p is expressed more than miR-155-5p; ii) antilipemic agent or a CSF-1R antagonist, and ii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons wherein miR-155-3p is expressed more than miR-155-5p; ii) antilipemic agent and a CSF-1R antagonist, and ii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function. In one embodiment, provided is a method of engrafting cells in vivo for therapeutic treatment, comprising, a) providing: i) a population of midbrain dopamine (DA) neurons wherein miR-155-3p is expressed more than miR-155-5p; ii) fenofibrate and pexidartinib, and ii) a subject, wherein said subject shows at least one neurological symptom; b) treating a subject with the antilipemic agent or CSF-1R antagonist; and c) transplanting said midbrain dopamine (DA) neurons into said subject under conditions for allowing in vivo engraftment and for providing dopamine (DA) neuronal function.

In one aspect disclosed is an in vitro cell population having a unique molecular profile in that the microRNA (miR or miRNA) and/or the miRNA profile of the cell population comprises more miR-155-3p compared to miR-155-5p or more modified miR-155-3p compared to un-modified miR-155-3p or more miR-155-5p compared to miR-155-3p or more modified miR-155-5p compared to un-modified miR-155-5p. In one aspect the in vitro cell compositions comprise, or alternatively consist essentially of, or yet further consist of, one or more, two or more, three or more, four or more, five or more, six or more, seven or more, eight or more, nine or more, ten or more, eleven or more, twelve or more, thirteen or more, fourteen or more, fifteen or more, sixteen or more, twenty or more, thirty or more, forty or more, fifty or more, sixty or more, seventy or more, eighty or more, ninety or more, ninety-five or more or ninety-nine or more times more miR-155-3p than miR-155-5p. In one aspect the in vitro cell compositions comprise, or alternatively consist essentially of, or yet further consist of, one or more, two or more, three or more, four or more, five or more, six or more, seven or more, eight or more, nine or more, ten or more, eleven or more, twelve or more, thirteen or more, fourteen or more, fifteen or more, sixteen or more, twenty or more, thirty or more, forty or more, fifty or more, sixty or more, seventy or more, eighty or more, ninety or more, ninety-five or more or ninety-nine or more times more miR-155-5p than miR-155-3p.

In further aspect, the cell populations of the above-noted compositions are identified by the microRNA (miR) profile by the lack of up-regulation of miR-155-5p compared to miR-155-3p. In further aspect, the cell populations of the above-noted compositions are identified by the microRNA (miR) profile by the lack of up-regulation of miR-155-3p compared to miR-155-5p.

The combination therapy is useful to perform methods for one or more of: a) inhibiting the progression of, b) preventing or c) treating, Parkinson's disease or an associated disorder in a subject in need thereof. The methods comprise, or alternatively consist essentially of, or yet further consist of, administering to the subject an effective amount of an antilipemic agent or a CSF-1R antagonist as described above along with a therapeutically effective amount of DA neuronal cells.

The therapy and patient's health can be monitored by determining the level of inflammatory response, during and after the therapy.

Also provided herein are isolated or purified cell populations isolated from a body fluid (e.g., urine, saliva, lymphatic fluid, breast milk, blood, serum and/or plasma) of a non-diseased subject or differentiated from an iPSC. In one aspect, the cell populations have a unique molecular profile in that the microRNA (miR) profile in the cell populations comprise the up-regulation of miR-155-3p or the up-regulation of miR-155-3p compared to miR-155-5p, or the up-regulation of miR-155-3p compared to wildtype.

Also provided herein are isolated or purified cell populations isolated from a body fluid (e.g., urine, saliva, lymphatic fluid, breast milk, blood, serum and/or plasma) of a non-diseased subject or differentiated from an iPSC. In one aspect, the cell populations have a unique molecular profile in that the microRNA (miR) profile in that the cell populations comprise the up-regulation of miR-155-5p or the up-regulation of miR-155-5p compared to miR-155-3p, or the up-regulation of miR-155-5p compared to wildtype.

### Compositions

### Unmodified DA Neural Cell Populations

Provided herein is a purified or isolated population of cells and a therapeutically effective amount of an antilipemic agent or CSF-1R antagonist. Provided herein is a purified or isolated population of cells and a therapeutically effective amount of an antilipemic agent. Provided herein is a purified or isolated population of cells and a therapeutically effective amount of fenofibrate. Provided herein is a purified or isolated population of cells and a therapeutically effective amount of fenofibrate. Provided herein is a purified or isolated population of cells and a therapeutically effective amount of a CSF-1R antagonist. Provided herein is a purified or isolated population of cells and a therapeutically effective amount of pexidartinib. In some embodiments, the purified or isolated population of cells comprise, consist essentially of, or consisting of DA neuronal cells. In some embodiments, the purified or isolated population of cells are differentiated from pluripotent cells.

Provided herein is a purified or isolated population of cells and a therapeutically effective amount of antilipemic agent and a CSF-1R antagonist. Provided herein is a purified or isolated population of cells and a therapeutically effective amount of fenofibrate and a CSF-1R antagonist. Provided herein is a purified or isolated population of cells and a therapeutically effective amount of fenofibrate and a CSF-1R antagonist. Provided herein is a purified or isolated population of cells and a therapeutically effective amount of fenofibrate and pexidartinib. Provided herein is a purified or isolated population of cells and a therapeutically effective amount of fenofibrate and pexidartinib. In some embodiments, the purified or isolated population of cells comprise, consist essentially of, or consisting of DA neuronal cells. In some embodiments, the purified or isolated population of cells are differentiated from pluripotent cells.

Paragraph 1. A composition for improving cell therapy in a subject, wherein the composition consists of a therapeutically effective amount of an antilipemic agent and DA neuronal cells.

Paragraph 2. A composition for improving cell therapy in a subject, wherein the composition consists of a therapeutically effective amount of fenofibrate and DA neuronal cells.

Paragraph 3. A composition for improving cell therapy in a subject, wherein the composition consists of a therapeutically effective amount of fenofibrate and DA neuronal cells.

Paragraph 4. A composition for improving cell therapy in a subject, wherein the comprises is a therapeutically effective amount of an antilipemic agent and DA neuronal cells.

Paragraph 5. A composition for improving cell therapy in a subject, wherein the comprises is a therapeutically effective amount of fenofibrate and DA neuronal cells.

Paragraph 6. A composition for improving cell therapy in a subject, wherein the comprises is a therapeutically effective amount of fenofibrate and DA neuronal cells.

Paragraph 7. The composition of any preceding paragraph wherein the composition does not comprise any of the formula or compounds in WO2014145051A1, US Patent Publication number 20170081326A1 and U.S. Pat. No. 10,717,735.

Paragraph 8. The composition of any preceding paragraph wherein the composition further comprises at least one pharmaceutically acceptable carrier.

Paragraph 9. The composition of any preceding paragraph wherein the composition further comprises at least one additional therapeutic agent.

Paragraph 10. The composition of any preceding paragraph wherein the composition further comprises a CSF-1R antagonist.

Paragraph 11. A kit comprising the composition of any preceding paragraph.

Paragraph 12. The composition of any preceding paragraph comprising a pharmaceutically acceptable salt of paragraphs 1-10.

Paragraph 13. The composition of any preceding paragraph wherein the DA neuronal cells are genetically modified.

Paragraph 14. The composition of any preceding paragraph wherein the DA neuronal cells are miR-155-3p biased or miR-155-5p biased.

Paragraph 15. A composition for improving cell therapy in a subject, wherein the composition consists of a therapeutically effective amount of fenofibrate and DA neuronal cells and not clorofibrate, benzafibrate and/or fenofibric acid.

### Modified DA Neural Cell Populations

Provided herein is a purified or isolated population of genetically modified cells and a therapeutically effective amount of an antilipemic agent or CSF-1R antagonist. Provided herein is a purified or isolated population of genetically modified cells and a therapeutically effective amount of an antilipemic agent. Provided herein is a purified or isolated population of genetically modified cells and a therapeutically effective amount of fenofibrate. Provided herein is a purified or isolated population of genetically modified cells and a therapeutically effective amount of fenofibrate. Provided herein is a purified or isolated population of genetically modified cells and a therapeutically effective amount of a CSF-1R antagonist. Provided herein is a purified or isolated population of genetically modified cells and a therapeutically effective amount of pexidartinib. In some embodiments, the purified or isolated population of genetically modified cells comprise, consist essentially of, or consisting of DA neuronal cells. In some embodiments, the purified or isolated population of genetically modified cells are differentiated from pluripotent cells.

Provided herein is a purified or isolated population of genetically modified cells and a therapeutically effective amount of an antilipemic agent and a CSF-1R antagonist. Provided herein is a purified or isolated population of genetically modified cells and a therapeutically effective amount of fenofibrate and a CSF-1R antagonist. Provided herein is a purified or isolated population of genetically modified cells and a therapeutically effective amount of fenofibrate and a CSF-1R antagonist. Provided herein is a purified or isolated population of genetically modified cells and a therapeutically effective amount of fenofibrate and pexidartinib. Provided herein is a purified or isolated population of genetically modified cells and a therapeutically effective amount of fenofibrate and pexidartinib. In some embodiments, the purified or isolated population of genetically modified cells comprise, consist essentially of, or consisting of DA neuronal cells. In some embodiments, the purified or isolated population of genetically modified cells are differentiated from pluripotent cells. In some embodiments, the DA neurons having modified pre-miR-155. In some embodiments, the DA neurons having a modified pre-miR-155 having SEQ ID No: 6. In some embodiments, the DA neurons having a modified pre-miR-155 having SEQ ID No: 7. In some embodiments, the DA neurons having a modified pre-miR-155 stem loop having SEQ ID No: 8. In some embodiments, the DA neurons having a modified pre-miR-155 having SEQ ID No: 6 and SEQ ID No: 7. In some embodiments, the DA neurons having SEQ ID No: 6, SEQ ID No: 7, and a pre-miR-155 stem loop having SEQ ID No: 8. In some embodiments, the DA neurons having a modified pre-miR-155 having SEQ ID No: 6 and a pre-miR-155 stem loop having SEQ ID No: 8. In some embodiments, the DA neurons having a modified pre-miR-155 having SEQ ID No: 7 and a pre-miR-155 stem loop having SEQ ID No: 8. SEQ ID Nos: 6-8 can be used to generate miR-155-3p biased or miR-155-5p biased DA neurons. In some embodiments, the DA neurons having a modified pre-miR-155 having SEQ ID No: 7 and a pre-miR-155 stem loop having SEQ ID No: 8. SEQ ID Nos: 6-8 can be used to generate miR-155-3p biased and miR-155-5p un-biased DA neurons. In some embodiments, the DA neurons having a modified pre-miR-155, which favors miR-155-3p strand selection. In some embodiments, the DA neurons having a modified pre-miR-155, which favors miR-155-5p strand selection. In some embodiments, the DA neurons having a modified pre-miR-155, which decreases miR-155-3p strand selection. In some embodiments, the DA neurons having a modified pre-miR-155, which decreases miR-155-5p strand selection. In some embodiments, the DA neurons having a modified pre-miR-155, which favors miR-155-3p strand selection and decreases miR-155-5p strand selection.

In certain aspects, disclosed is a pre-miRNA-155 sequence, wherein the pre-miRNA-155 incorporated into a DA neuronal cell has an anti-inflammatory effect on human subjects. In certain aspects, disclosed is a pre-miRNA-155 sequence, wherein the pre-miRNA-155 incorporated into a DA neuronal cell has a pro-inflammatory effect on human subjects.

These compositions are useful for the treatment of disease, such as neurologic disease and associated disorders.

In some embodiments, described herein, the cell population is differentiated using a mono-SMAD, or dual-SMAD method.

Sequence IDs

SEQ ID No 1: >human hsa-miR-155-5p MIMAT0000646
UUAAUGCUAAUCGUGAUAGGGGUU
SEQ ID No 2: >mouse miR-155-5p MIMAT0000646
UUAAUGCUAAUUGUGAUAGGGGUU
SEQ ID No 3: >human hsa-miR-155-3p MIMAT0004658
CUCCUACAUAUUAGCAUUAACA
SEQ ID No 4: >mouse -miR-155-3p MIMAT0004658
CUCCUACCUGUUAGCAUUAACA
SEQ ID No 5: >hsa-miR-155 stem loop MI0000681
CUGUUAAUGCUAAUCGUGAUAGGGGUUUUUGCCUCCAACUGACUCCUACA
UAUUAGCAUUAACAG
SEQ ID No 6: >modified hsa-miR-155-5p
GGAAUGCUAAUCGUGAUAGGGGUU
SEQ ID No 7: >modified hsa-miR-155-3p
UUCCUACAUAUUAGCAUUAACA
SEQ ID No 8: >modified hsa-miR-155 stem loop
CUGUUAAUGCUAAUCGUGAUAGGGAUUUUUGCCUCCAACUGAUUCCUACA
UAUUAGCAUUAACAG
SEQ ID No 9: >miR-155-3p isomiR
3′-ACC**UACGAUUAUACAUCCUC
SEQ ID No 10: >miR-155-3p isomiR
3′-*CAAUUACGAUUAUACAUCCU*
SEQ ID No 11: >miR-155-3p isomiR
3′-ACAAUUACGAUUAUACAUCCU*
SEQ ID No 12: >miR-155-3p isomiR
3′-***ACUACGAUUAUACAUCCUC
SEQ ID No 13: >miR-155-3p isomiR
3′-**AAUUACGAUUAUACAUCCU*
SEQ ID No 14: >miR-155-3p isomiR
3′-***AUUACGAUUAUACAUCCUC
SEQ ID No 15: >miR-155-3p isomiR
3′-******ACGAUUAUACAUCCUCA
SEQ ID No 16: >miR-155-3p isomiR
3′-******ACGAUUAUACAUCCUCAG
SEQ ID No 17: >miR-155-3p isomiR
3′-*CAA*UACGAUUAUACAUCCUC
SEQ ID No 18: >miR-155-3p isomiR
3′-***AUUACGAUUAUACAUCCUCA
SEQ ID No 19: >miR-155-3p isomiR
3′-***CUUACGAUUAUACAUCCUC
SEQ ID No 20: >miR-155-3p isomiR
3′-ACAAUUACGAUUAUACAU
SEQ ID No 21: >miR-155-3p isomiR
3′-****UUACGAUUAUACAUCCUC
All * in the sequence listings represent deletions.

### DA Neural Cell Populations

In some embodiments, the DA neural cell population described herein comprises three distinct cell populations: (1) A9 dopamine neurons (2) Astrocytes and (3) vascular leptomeningeal cells (VLMC).

Disclosed is a neuronal cell population comprising, consisting essentially of, or consisting of A9 dopamine neurons, Astrocytes or VLMC and an antilipemic agent or CSF-1R antagonist. A neuronal cell population comprising, consisting essentially of, or consisting of A9 dopamine neurons, Astrocytes or VLMC and an antilipemic agent. A neuronal cell population comprising, consisting essentially of, or consisting of A9 dopamine neurons, Astrocytes or VLMC and fenofibrate. A neuronal cell population comprising, consisting essentially of, or consisting of A9 dopamine neurons, Astrocytes or VLMC and fenofibrate. A neuronal cell population comprising, consisting essentially of, or consisting of A9 dopamine neurons, Astrocytes or VLMC and a CSF-1R antagonist. A neuronal cell population comprising, consisting essentially of, or consisting of A9 dopamine neurons, Astrocytes or VLMC and pexidartinib.

A neuronal cell population comprising, consisting essentially of, or consisting of A9 dopamine neurons, Astrocytes or VLMC and an antilipemic agent and a CSF-1R antagonist. A neuronal cell population comprising, consisting essentially of, or consisting of A9 dopamine neurons, Astrocytes or VLMC and fenofibrate and a CSF-1R antagonist. A neuronal cell population comprising, consisting essentially of, or consisting of A9 dopamine neurons, Astrocytes or VLMC and fenofibrate and a CSF-1R antagonist. A neuronal cell population comprising, consisting essentially of, or consisting of A9 dopamine neurons, Astrocytes or VLMC and fenofibrate and pexidartinib. A neuronal cell population comprising, consisting essentially of, or consisting of A9 dopamine neurons, Astrocytes or VLMC and fenofibrate and pexidartinib.

### Pharmaceutical Compositions

This disclosure also provides A pharmaceutical composition comprising, or alternatively consisting essentially of, or yet further consisting of, a pharmaceutically acceptable carrier and an effective amount of an antilipemic agent or CSF-1R antagonist. A pharmaceutical composition comprising, or alternatively consisting essentially of, or yet further consisting of, a pharmaceutically acceptable carrier and an effective amount of an antilipemic agent. pharmaceutical compositions comprising, or alternatively consisting essentially of, or yet further consisting of, a pharmaceutically acceptable carrier and an effective amount of fenofibrate. A pharmaceutical composition comprising, or alternatively consisting essentially of, or yet further consisting of, a pharmaceutically acceptable carrier and an effective amount of fenofibrate. A pharmaceutical composition comprising, or alternatively consisting essentially of, or yet further consisting of, a pharmaceutically acceptable carrier and an effective amount of a CSF-1R antagonist. pharmaceutical compositions comprising, or alternatively consisting essentially of, or yet further consisting of, a pharmaceutically acceptable carrier and an effective amount of pexidartinib. In some embodiments, the cells differentiated from pluripotent cells are modified DA neural cells. In some embodiments, the cells differentiated from pluripotent cells are un-modified DA neural cells.

A pharmaceutical composition comprising, or alternatively consisting essentially of, or yet further consisting of, a pharmaceutically acceptable carrier and an effective amount of an antilipemic agent and a CSF-1R antagonist. A pharmaceutical composition comprising, or alternatively consisting essentially of, or yet further consisting of, a pharmaceutically acceptable carrier and an effective amount of fenofibrate and a CSF-1R antagonist. A pharmaceutical composition comprising, or alternatively consisting essentially of, or yet further consisting of, a pharmaceutically acceptable carrier and an effective amount of fenofibrate and a CSF-1R antagonist. A pharmaceutical composition comprising, or alternatively consisting essentially of, or yet further consisting of, a pharmaceutically acceptable carrier and an effective amount of fenofibrate and pexidartinib. A pharmaceutical composition comprising, or alternatively consisting essentially of, or yet further consisting of, a pharmaceutically acceptable carrier and an effective amount of fenofibrate and pexidartinib. In some embodiments, the cells differentiated from pluripotent cells are modified DA neural cells. In some embodiments, the cells differentiated from pluripotent cells are un-modified DA neural cells.

The compositions can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. Non-limiting examples of carriers include phosphate buffered saline (PBS), saline or a biocompatible matrix material. The compositions can contain a protease inhibitor, glycerol and/or dimethyl sulfoxide (DMSO).

The pharmaceutical compositions can be conveniently presented in dosage unit form and can be prepared by any of the methods well known in the art of pharmacy. In the pharmaceutical composition the active object compound is included in an amount sufficient to produce the desired therapeutic effect. For example, pharmaceutical compositions of the disclosure (an antilipemic agent or a CSF-1R antagonist) may take a form suitable for virtually any mode of administration, including, for example, topical, ocular, oral, buccal, systemic, nasal, injection, transdermal, rectal, and vaginal, or a form suitable for administration by inhalation or insufflation.

Systemic formulations include those designed for administration by injection (e.g., subcutaneous, intravenous, intramuscular, intrathecal, or intraperitoneal injection) as well as those designed for transdermal, transmucosal, oral, or pulmonary administration.

The compositions will generally be used in an amount effective to achieve the intended result, for example, in an amount effective to treat or prevent the particular condition being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also includes halting or slowing the progression of the disease, regardless of whether improvement is realized.

The amount of compound administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the disclosure will also depend on the age, weight, general health, and severity of the condition of the individual being treated. Dosage may also need to be tailored to the sex of the individual and/or the lung capacity of the individual, where administered by inhalation. Dosage, and frequency of administration of the compositions will also depend on whether the compositions are formulated for treatment of acute episodes of a condition or for the prophylactic treatment of a disorder. A skilled practitioner will be able to determine the optimal dose for a particular individual.

For prophylactic administration, the compound can be administered to a patient at risk of developing one of the previously described conditions. For example, if it is unknown whether a patient is allergic to a particular drug, the compound can be administered prior to administration of the drug to avoid or ameliorate an allergic response to the drug. Alternatively, prophylactic administration can be applied to avoid the onset of symptoms in a patient diagnosed with the underlying disorder.

Effective dosages can be estimated initially from in vitro assays. For example, an initial dosage for use in animals can be formulated to achieve a therapeutic concentration and/or dosage of the miR-155-3p biased cell composition or miR-155-5p biased cell composition, as measured in an in vitro assay. Calculating dosages to achieve such effective dosages for other animal models or human patients is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-446, latest edition, Pergamagon Press, and the references cited therein.

Initial dosages can also be estimated from in vivo data, such as animal models. Animal models useful for testing the efficacy of compounds to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 1000 mg/kg/day, but can be higher or lower, depending upon, among other factors, the activity of the composition, its bioavailability, the mode of administration, and various factors discussed above. Dosage amount and interval can be adjusted individually to provide local and/or systemic concentration of the cell populations that are sufficient to maintain therapeutic or prophylactic effect. For example, the compositions can be administered once per week, several times per week (e.g., every other day), once per day, or multiple times per day, depending upon, among other things, the mode of administration, the specific indication being treated, and the judgment of the prescribing physician. Skilled artisans will be able to optimize effective local dosages without undue experimentation.

Preferably, the compound(s) will provide therapeutic or prophylactic benefit without causing substantial toxicity. Toxicity of the compounds can be determined using standard pharmaceutical procedures. The dose ratio between toxic and therapeutic (or prophylactic) effect is the therapeutic index. Compositions that exhibit high therapeutic indices are preferred.

Aspects and embodiments of the methods disclosed herein can be further understood by in the following numbered paragraphs:

Paragraph 1: A method of enhancing cell engraftment in a mammal comprising treating the mammal with an antilipemic agent.

Paragraph 2: A method of enhancing cell engraftment in a mammal comprising treating the mammal with fenofibrate.

Paragraph 3: A method of enhancing cell engraftment in a mammal comprising treating the mammal with fenofibrate.

Paragraph 4: A method of enhancing cell engraftment in a mammal comprising treating the mammal with a CSF-1R antagonist.

Paragraph 5: A method of enhancing cell engraftment in a mammal comprising treating the mammal with pexidartinib.

Paragraph 6: A method of enhancing cell engraftment in a mammal comprising treating the mammal with an antilipemic agent and a CSF-1R antagonist.

Paragraph 7: A method of enhancing cell engraftment in a mammal comprising treating the mammal with fenofibrate and a CSF-1R antagonist.

Paragraph 8: A method of enhancing cell engraftment in a mammal comprising treating the mammal with fenofibrate and a CSF-1R antagonist.

Paragraph 9: A method of enhancing cell engraftment in a mammal comprising treating the mammal with fenofibrate and pexidartinib.

Paragraph 10: A method of enhancing cell engraftment in a mammal comprising treating the mammal with fenofibrate and pexidartinib.

Paragraph 11: The method of any preceding paragraph further comprising administering cells for engraftment to the patient.

Paragraph 12: The method of any preceding paragraph further comprising administering DA neuronal cells for engraftment to the patient.

Paragraph 13: The method of paragraph 12, wherein the DA neuronal cells are contacted with an antilipemic agent and/or a CSF-1R antagonist prior to engraftment.

Paragraph 14: The method of any preceding paragraph further comprising administering DA neuronal cells and pluripotent cells for engraftment to the patient.

Paragraph 15: The method of any preceding paragraph further comprising administering cells for engraftment to the patient wherein the cells are administered to healthy tissue.

Paragraph 16: The method of any paragraphs 1-15 further comprising administering cells for engraftment to the patient wherein the cells are administered to un-healthy tissue.

Paragraph 17: The method of any preceding paragraph further comprising administering cells for engraftment to the patient wherein the cells are administered to the mammal's brain.

Paragraph 18: A method of enhancing cell engraftment in a mammal comprising treating the mammal with fenofibrate and not clorofibrate, benzafibrate and/or fenofibric acid.

### Pharmaceutical Compositions+Un-Modified Cells

This disclosure also provides pharmaceutical compositions comprising, or consisting essentially of, or yet further consisting of, purified or isolated DA neuronal cell populations and an antilipemic agent and/or CSF-1R antagonist. In one aspect, the pharmaceutical composition comprises, or alternatively consists essentially of, or yet further consists of, a pharmaceutically acceptable carrier and an effective amount of a DA neuronal cell population and an antilipemic agent and/or CSF-1R antagonist.

### Pharmaceutical Compositions+Modified Cells

This disclosure also provides pharmaceutical compositions comprising, or consisting essentially of, or yet further consisting of, purified or isolated miR-155-3p biased DA cell populations or miR-155-5p biased DA cell populations and an antilipemic agent and/or CSF-1R antagonist. In one aspect, the pharmaceutical composition comprises, or alternatively consists essentially of, or yet further consists of, a pharmaceutically acceptable carrier and an effective amount of these miR-155-3p biased DA cell populations or miR-155-5p biased DA cell populations and an antilipemic agent and/or CSF-1R antagonist.

Non-limiting examples of carriers include phosphate buffered saline (PBS), saline or a biocompatible matrix material such as a collagen matrix. The compositions can contain a protease inhibitor, glycerol and/or dimethyl sulfoxide (DMSO). The pharmaceutically acceptable carrier comprises one or more of a biocompatible matrix or a liquid carrier. The pharmaceutical compositions of this disclosure can be formulated for freeze-drying or lyophilization using methods known in the art.

The pharmaceutical compositions are intended for in vitro and in vivo use. The compositions can comprise a concentration of miR-155-3p biased DA cell populations or miR-155-5p biased DA cell populations from about 1 mg/ml to about 10 mg/ml, or alternatively from about 1 to about 8 mg/ml, or alternatively from 2 to about 8 mg/ml, or alternatively from 2 to about 5 mg/ml, or about 2 to 4 mg/ml, or alternatively from 3 mg/ml to 20 mg/ml. When administered to the subject, an effective amount of the miR-155-3p biased DA cell populations or miR-155-5p biased DA cell populations are administered to the subject, to cause at least about 5%, or alternatively at least about 10%, or alternatively at least about 20%, or alternatively at least about 30%, or alternatively at least about 40%, or alternatively at least about 50%, or alternatively at least about 60%, or alternatively at least about 70%, or alternatively at least about 80%, or alternatively at least about 85%, or alternatively at least about 90%, or alternatively at least about 95%, or alternatively at least about 99% effectiveness in the methods provided herein as compared to a control that does not receive the composition. Comparative effectiveness can be determined by suitable in vitro or in vivo methods as known in the art and briefly exemplified herein.

In one aspect, the compositions are pharmaceutical formulations for use in the therapeutic methods of this disclosure and for the treatment of the appropriate or relevant disease. While the examples are noted for the treatment of PD, the principles can be applied to other disease conditions, including neurologic diseases.

In a further aspect, the disclosure provides a pharmaceutical composition comprising, or alternatively consisting essentially of, or yet further consisting of, the isolated or purified miR-155-3p biased DA cell populations or miR-155-5p biased DA cell populations in a concentration such that composition comprises at least 75%, or alternatively at least 80%, or alternatively at least 85%, or alternatively at least 90%, or alternatively at least 95% or alternatively at least 97%, or alternatively at least 98%, or alternatively, at least 99% of cells in the total composition and an antilipemic agent and/or CSF-1R antagonist.

Cell populations may be selected such that more than about 50% (alternatively more than about 60%, more than about 70%, more than about 80%, more than about 90% or even more than about 95%) of the cells express at least one, at least two, at least three, at least four, at least five of the markers for DA neurons or at least one, at least two, at least three, at least four, at least five of the markers for DA neural cells. DA neural cell markers include but are not limited to: FOXA2, LMX1A, NURR1, TH, OTX2, Tuj1, TTF3, PITX3, ASCL, EBF-1, EBF-3, TTR, DAT, Kir3.2/GIRK2, CD 142, DCSM1, CD63, and CD99

Isolated cell populations obtained according to the methods describe herein are typically non-homogeneous, although homogeneous cell populations are also contemplated.

According to a particular embodiment, the cell populations are genetically modified to express an exogenous miRNA or a polynucleotide agent capable of down-regulating the miRNA. In some embodiments, the modified miRNA comprises SEQ ID Nos. 6, 7 or 8. In some embodiments, the modified miRNA comprises a modification of miR-155-3p or miR-155-5p in a pluripotent cell population.

Cell populations may be selected such that more than about 50% (alternatively more than about 60%, more than about 70%, more than about 80%, more than about 90% or even more than about 95%) of the cells have a modified miRNA such as miR-155-3p or miR-155-5p.

Isolation of particular subpopulations of cells may be affected using techniques known in the art including fluorescent activated cell sorting and/or magnetic separation of cells.

The cell populations may comprise DA neurons or DA neuronal cell phenotypes including a cell size, a cell shape, an organelle size and an organelle number. These structural phenotypes may be analyzed using microscopic techniques (e.g. scanning electron microscopy). Antibodies or dyes may be used to highlight distinguishing features in order to aid in the analysis.

In some embodiments, the present disclosure provides a composition comprising, consisting of, or consisting essentially of at least one antilipemic agent and/or at least one CSF-1R antagonist, and one or more other therapeutic agents. In some embodiments, the one or more other therapeutic agents are selected from an alkylating agent, including, but not limiting to, adozelesin, altretamine, bendamustine, bizelesin, busulfan, carboplatin, carboquone, carmofur, carmustine, chlorambucil, cisplatin, cyclophosphamide, dacarbazine, estramustine, etoglucid, fotemustine, hepsulfam, ifosfamide, improsulfan, irofulven, lomustine, mannosulfan, mechlorethamine, melphalan, mitobronitol, nedaplatin, nimustine, oxaliplatin, piposulfan, prednimustine, procarbazine, ranimustine, satraplatin, semustine, streptozocin, temozolomide, thiotepa, treosulfan, triaziquone, triethylenemelamine, triplatin tetranitrate, trofosphamide, and uramustine; an antibiotic, including, but not limiting to, aclarubicin, amrubicin, bleomycin, dactinomycin, daunorubicin, doxorubicin, elsamitrucin, epirubicin, idarubicin, menogaril, mitomycin, neocarzinostatin, pentostatin, pirarubicin, plicamycin, valrubicin, and zorubicin; an antimetabolite, including, but not limiting to, aminopterin, azacitidine, azathioprine, capecitabine, cladribine, clofarabine, cytarabine, decitabine, floxuridine, fludarabine, 5-fluorouracil, gemcitabine, hydroxyurea, mercaptopurine, methotrexate, nelarabine, pemetrexed, raltitrexed, tegafur-uracil, thioguanine, trimethoprim, trimetrexate, and vidarabine; an immunotherapy, an antibody therapy, including, but not limiting to, alemtuzumab, bevacizumab, cetuximab, galiximab, gemtuzumab, panitumumab, pertuzumab, rituximab, brentuximab, tositumomab, trastuzumab, 90 Y ibritumomab tiuxetan, ipilimumab, tremelimumab and anti-CTLA-4 antibodies; a hormone or hormone antagonist, including, but not limiting to, anastrozole, androgens, buserelin, diethylstilbestrol, exemestane, flutamide, fulvestrant, goserelin, idoxifene, letrozole, leuprolide, magestrol, raloxifene, tamoxifen, and toremifene; a taxane, including, but not limiting to, DJ-927, docetaxel, TPI 287, larotaxel, ortataxel, paclitaxel, DHA-paclitaxel, and tesetaxel; a retinoid, including, but not limiting to, alitretinoin, bexarotene, fenretinide, isotretinoin, and tretinoin; an alkaloid, including, but not limiting to, demecolcine, homoharringtonine, vinblastine, vincristine, vindesine, vinflunine, and vinorelbine; an antiangiogenic agent, including, but not limiting to, AE-941 (GW786034, Neovastat), ABT-510, 2-methoxyestradiol, lenalidomide, and thalidomide; a topoisomerase inhibitor, including, but not limiting to, amsacrine, belotecan, edotecarin, etoposide, etoposide phosphate, exatecan, irinotecan (also active metabolite SN-38 (7-ethyl-10-hydroxy-camptothecin)), lucanthone, mitoxantrone, pixantrone, rubitecan, teniposide, topotecan, and 9-aminocamptothecin; a kinase inhibitor, including, but not liming to, axitinib (AG 013736), dasatinib (BMS 354825), erlotinib, gefitinib, flavopiridol, imatinib mesylate, lapatinib, motesanib diphosphate (AMG 706), nilotinib (AMN107), seliciclib, sorafenib, sunitinib malate, AEE-788, BMS-599626, UCN-01 (7-hydroxystaurosporine), vemurafenib, dabrafenib, selumetinib, LGX818, BGB-283, and vatalanib; a targeted signal transduction inhibitor including, but not limiting to bortezomib, geldanamycin, and rapamycin; a biological response modifier, including, but not limiting to, imiquimod, interferon-α, and interleukin-2; and other chemotherapeutics, including, but not limiting to 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, mTOR inhibitors (e.g. sirolimus, temsirolimus, everolimus, deforolimus), PI3K inhibitors (e.g. BEZ235, GDC-0941, XL147, XL765, •BMK120), Cdk4 inhibitors (e.g. PD-332991), Akt inhibitors, Hsp90 inhibitors (e.g. geldanamycin, radicicol, tanespimycin), farnesyltransferase inhibitors (e.g. tipifarnib), and Aromatase inhibitors (anastrozole letrozole exemestane). In one embodiment, the method of treating a cancer involves administering to the subject an effective amount of a composition including any one or more compound(s) of Formulae (I) or (II); or a pharmaceutically acceptable salt, a solvate, a tautomer, an isomer, or a deuterated analog of Formulae (I) or (II); or any of the compounds in Table I, in combination with a chemotherapeutic agent selected from capecitabine, 5-fluorouracil, carboplatin, dacarbazine, gefitinib, oxaliplatin, paclitaxel, SN-38, temozolomide, vinblastine, bevacizumab, cetuximab, interferon-α, interleukin-2, or erlotinib. In another embodiment, the chemotherapeutic agent is a Mek inhibitor. Exemplary Mek inhibitors include, but are not limited to, AS703026, AZD6244 (Selumetinib), AZD8330, BIX 02188, CI-1040 (PD184352), GSK1120212 (JTP-74057), PD0325901, PD318088, PD98059, RDEA119(BAY 869766), TAK-733 and U0126-EtOH. In another embodiment, the chemotherapeutic agent is a tyrosine kinase inhibitor. Exemplary tyrosine kinase inhibitors include, but are not limited to, AEE788, AG-1478 (Tyrphostin AG-1478), AG-490, Apatinib (YN968D1), AV-412, AV-951(Tivozanib), Axitinib, AZD8931, BIBF1120 (Vargatef), BIBW2992 (Afatinib), BMS794833, BMS-599626, Brivanib (BMS-540215), Brivanib alaninate(BMS-582664), Cediranib (AZD2171), Chrysophanic acid (Chrysophanol), Crenolanib (CP-868569), CUDC-101, CYC116, Dovitinib Dilactic acid (TKI258 Dilactic acid), E7080, Erlotinib Hydrochloride (Tarceva, CP-358774, OSI-774, NSC-718781), Foretinib (GSK1363089, XL880), Gefitinib (ZD-1839 or Iressa), Imatinib (Gleevec), Imatinib Mesylate, Ki8751, KRN 633, Lapatinib (Tykerb), Linifanib (ABT-869), Masitinib (Masivet, AB1010), MGCD-265, Motesanib (AMG-706), MP-470, Mubritinib (TAK 165), Neratinib (HKI-272), NVP-BHG712, OSI-420 (Desmethyl Erlotinib,CP-473420), OSI-930, Pazopanib HCl, PD-153035 HCl, PD173074, Pelitinib (EKB-569), PF299804, Ponatinib (AP24534), PP121, RAF265 (CHIR-265), Raf265 derivative, Regorafenib (BAY 73-4506), Sorafenib Tosylate (Nexavar), Sunitinib Malate (Sutent), Telatinib (BAY 57-9352), TSU-68 (SU6668), Vandetanib (Zactima), Vatalanib dihydrochloride (PTK787), WZ3146, WZ4002, WZ8040, quizartinib, Cabozantinib, XL647, EGFR siRNA, FLT4 siRNA, KDR siRNA, Antidiabetic agents such as metformin, and DPP4 inhibitors (sitagliptin, vildagliptin, saxagliptin, dutogliptin, gemigliptin, alogliptin). In another embodiment, the agent is an EGFR inhibitor. Exemplary EGFR inhibitors include, but are not limited to, AEE-788, AP-26113, BIBW-2992 (Tovok), CI-1033, GW-572016, Iressa, LY2874455, RO-5323441, Tarceva (Erlotinib, OSI-774), CUDC-101 and WZ4002. In another embodiment, the therapeutic agent for combination is a c-Fms and/or c-Kit inhibitor as described in US Patent Application Publication Nos. 2009/0076046 and 2011/0112127, which are incorporated herein by reference in their entirety and for all purposes. In one embodiment, the method of treating a cancer involves administering to the subject an effective amount of a composition including any one or more compound(s) as described herein in combination with a chemotherapeutic agent selected from capecitabine, 5-fluorouracil, carboplatin, dacarbazine, gefitinib, oxaliplatin, paclitaxel, SN-38, temozolomide, vinblastine, bevacizumab, cetuximab, interferon-α, interleukin-2, or erlotinib. In some embodiments, the present disclosure provides a composition comprising, consisting of, or consisting essentially of an antilipemic agent and/or a CSF-1R antagonist, one or more other therapeutic agents and DA neuronal cells.

Disclosed is a DA neuronal cell population comprising, consisting essentially of, or consisting of an antilipemic agent and/or CSF-1R antagonist and wherein the DA neuronal cell population is a genetically modified cell population. Disclosed is a DA neuronal cell population comprising, consisting essentially of, or consisting of an antilipemic agent and/or CSF-1R antagonist and wherein the level of miR-155-3p (5′CUCCUACAUAUUAGCAUUAACA3′) (SEQ ID NO: 3) or a variant thereof is increased or decreased compared to wildtype. Disclosed is a DA neuronal cell population comprising, consisting essentially of, or consisting of an antilipemic agent and/or CSF-1R antagonist and wherein the level of miR-155-5p (5′GGAAUGCUAAUCGUGAUAGGGGUU3′) (SEQ ID NO: 6) or a variant thereof is increased or decreased compared to wildtype. A DA neuronal cell population comprising, consisting essentially of, or consisting of an antilipemic agent and/or CSF-1R antagonist and wherein the level of miR-155-5p (5′UUAAUGCUAAUCGUGAUAGGGGUU3′) (SEQ ID NO: 1) or a variant thereof is increased or decreased compared to miR-155-3p (5′UUCCUACAUAUUAGCAUUAACA3′) (SEQ ID NO: 7). A DA neuronal cell population comprising, consisting essentially of, or consisting of an antilipemic agent and/or CSF-1R antagonist and wherein the level of miR-155-3p (5′UUCCUACAUAUUAGCAUUAACA3′) (SEQ ID NO: 7) or a variant thereof is increased or decreased compared to miR-155-5p (5′UUAAUGCUAAUCGUGAUAGGGGUU3′) (SEQ ID NO: 1).

Disclosed is a composition comprising, consisting essentially of, or consisting of an antilipemic agent and/or CSF-1R antagonist and an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-5p enhanced midbrain dopamine (DA) neurons. Disclosed is a composition comprising, consisting essentially of, or consisting of an antilipemic agent and/or CSF-1R antagonist and an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-5p or miR-155-3p modified midbrain dopamine (DA) neurons. Disclosed is a composition comprising, consisting essentially of, or consisting of an antilipemic agent and/or CSF-1R antagonist and an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-3p biased midbrain dopamine (DA) neurons. Disclosed is a composition comprising, consisting essentially of, or consisting of an antilipemic agent and/or CSF-1R antagonist and an in vitro cell population wherein the majority of cells in said cell population are tyrosine hydroxylase (TH)+, forkhead box protein A2 (FOXA2)+, LIM homeobox transcription factor 1+, alpha (LMX1A)+ floor plate miR-155-5p biased midbrain dopamine (DA) neurons.

### Isolation of Pluripotent Cells

The embryonic stem cells of some embodiments can be obtained using well-known cell-culture methods. For example, human embryonic stem cells can be isolated from human blastocysts. Human blastocysts are typically obtained from human in vivo pre-implantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., U.S. Pat. No. 5,843,780; Science 282: 1145, 1998; Curr. Top. Dev. Biol. 38: 133, 1998; Proc. Natl. Acad. Sci. USA 92: 7844, 1995; Bongso et al., Hum Reprod 4: 706, 30 1989]; and Gardner et al., Fertil. Steril. 69: 84, 1998].

It will be appreciated that commercially available stem cells can also be used. Human ES cells can be purchased from the NIH human embryonic stem cells registry (www.escr.nih.gov). Non-limiting examples of commercially available embryonic stem cell lines are BGO1, BG02, BG03, BG04, CY12, CY30, CY92, CY10, TE03 and TE32.

In addition, ES cells can be obtained from other species as well, including mouse (Mills and Bradley, 2001), golden hamster Doetschman et al., 1988, Dev Biol. 127: 224-7], rat Iannaccone et al., 1994, Dev Biol. 163: 288-92] rabbit Giles et al. 1993, Mol Reprod Dev. 36: 130-8; Graves & Moreadith, 1993, Mol Reprod Dev. 1993, 36: 424-33], several domestic animal species Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) Thomson et al., 1995, Proc Natl Acad Sci USA. 92: 7844-8; Thomson et al., 1996, Biol Reprod. 55: 254-9].

Induced pluripotent stem cells (iPS) (embryonic-like stem cells) can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872. Other embryonic-like stem cells can be generated by nuclear transfer to oocytes, fusion with embryonic stem cells or nuclear transfer into zygotes if the recipient cells are arrested in mitosis.

The pluripotent cells (hES or iPSC) used herein may be of autologous, syngeneic or allogeneic related (matched siblings or haploidentical family members) or unrelated fully mismatched source.

### Culturing Pluripotent Cells

Culturing of pluripotent cells can be performed in any media that supports pluripotent cells which are known in the art. Methods for preparing and culturing pluripotent stem cells such as ES cells can be found in standard textbooks and reviews in cell biology, tissue culture, and embryology, including teratocarcinomas and embryonic stem cells: Guide to Techniques in Mouse Development (1993); Embryonic Stem Cell Differentiation in vitro (1993); Properties and uses of Embryonic Stem Cells: Prospects for Application to Human Biology and Gene Therapy (1998), all incorporated herein by reference. Standard methods used in tissue culture generally are described in Animal Cell Culture (1987); Gene Transfer Vectors for Mammalian Cells (1987); and Current Protocols in Molecular Biology and Short Protocols in Molecular Biology (1987 & 1995).

As mentioned, the cultured pluripotent cells can be modified. The cultured modified pluripotent cells have at least one modified miRNAs such as pre-mir155-5p or pre-mir155-3p. The cultured modified pluripotent cells have at least one modified miRNAs such as pre-mir155-5p or pre-mir155-3p in order to induce differentiation towards miR-155-3p biased or miR-155-5p biased DA neurons. The cultured modified pluripotent cells have at least one modified miRNAs such as pre-mir155-5p or pre-mir155-3p in order to induce differentiation towards miR-155-3p biased and/or miR-155-5p un-biased DA neurons.

### Differentiation Methods

Another aspect of the present disclosure relates to a culture of modified or unmodified midbrain dopaminergic (DA) neurons generated by a mono-SMAD or Dual-SMAD method described above from pluripotent cells and an antilipemic agent and/or a CSF-1R antagonist. The culture may further be comprised in a container means. The neurons may be comprised in a pharmaceutical preparation such as, e.g., a pharmaceutical preparation formulated for injection into a host which has been treated with an antilipemic agent and/or a CSF-1R antagonist.

During or following the differentiation step the pluripotent cells may be monitored for their differentiation state. Cell differentiation can be determined upon examination of cell or tissue-specific markers which are known to be indicative of differentiation.

Below is a list of markers that may be used to confirm differentiation into DA neurons: FOXA2 or LMX1. Additional markers include TH, rthodenticle homeobox 2 (OTX2), nuclear receptor related 1 protein (NURR1), Neuron-specific class III beta-tubulin (Tuj1), TTF3, paired-like homeodomain 3 (PITX3), achaete-scute complex (ASCL), early B-cell factor 1 (EBF-1), early B-cell factor 3 (EBF-3), transthyretin (TTR), synapsin, dopamine transporter (DAT), G-protein coupled, inwardly rectifying potassium channel (Kir3.2/GIRK2), CD142, DCSM1, CD63, and CD99.

Tissue/cell specific markers can be detected using immunological techniques well known in the art Thomson J A et al., (1998). Science 282: 1145-7. Examples include, but are not limited to, flow cytometry for membrane-bound markers, immunohistochemistry for extracellular and intracellular markers and enzymatic immunoassay, for secreted molecular markers.

It will be appreciated that the cells obtained according to the methods described herein may be enriched for a particular cell type—e.g. progenitor cell type or mature cell type. Thus for example, the time of differentiation may be selected to obtain an earlier progenitor type or a later mature cell type.

Disclosed are methods of ex vivo differentiating pluripotent cells towards neural progenitor cells and DA neurons using microRNAs.

Numerous methods are known in the art for differentiating pluripotent cells to DA neuronal cells including genetic modification and/or culturing in a medium which promotes differentiation towards that fate. The medium typically comprises at least one activator of Sonic hedgehog (SHH) signaling, and at least one activator of wingless (Wnt) signaling. Typically, the differentiation is affected in serum free medium, or serum replacements.

The present disclosure relates to the field of stem cell biology, in particular the lineage specific differentiation of pluripotent or multipotent stem cells, which can include, but is not limited to, human embryonic stem cells (hESC) in addition to nonembryonic induced pluripotent stem cells (iPSC), somatic stem cells, stem cells from patients with a disease, or any other cell capable of lineage specific differentiation. Specifically described are methods to direct the lineage specific differentiation of hESC and/or iPSC into floor plate midbrain progenitor cells and then further into large populations of midbrain fate FOXA2+LMX1A+TH+ dopamine (DA) neurons using novel culture conditions. The midbrain fate FOXA2+LMX1A+TH+ dopamine (DA) neurons made using the methods disclosed herein are further contemplated for various uses including, but not limited to, use in in vitro drug discovery assays, neurology research, and as a therapeutic to reverse disease of, or damage to, a lack of dopamine neurons in a patient. Further, compositions and methods are provided for differentiating midbrain fate FOXA2+LMXTA+TH+ dopamine (DA) neurons from human pluripotent stem cells for use in disease modeling, in particular Parkinson's disease.

As discussed above, differentiation of pluripotent stem cells to DA Neurons is known to those of skill in the art. See U.S. Pat. No. 10,280,398 and 10,590,383. Table 1 below is a single representative example of differentiation of pluripotent stem cells to DA Neurons. Table 1 below is a reproduction of Table 6, condition 9 from U.S. Pat. No. 10,590,383. The cell population at day 17 (D17) results in FoxA2+/Lmx1+DA progenitor cells. When the iPSC cells are modified as described herein, the cell population at day 17 (D17) results in FoxA2+/Lmx1+ modified DA progenitor cells.

TABLE 1
1. Condition 9 (200 nM LDN)

| | | | | |
|---|---|---|---|---|
| Component | Vendor | Cat# | Stock | Final Conc. |

E8 Plating Medium (Day −2)

| Essential 8 Basal Medium | Life Technologies | A14666SA | lx | | 98% | |
|---|---|---|---|---|---|---|
| Essential 8 Supplement | Life Technologies | A146665A | 50x | | 2% | |
| H1152 | CDI | H024 | 100 | μM | 1 | μM |

E8 Medium (Days −1 and 0)

| | | | | |
|---|---|---|---|---|
| Essential 8 Basal Medium | Life Technologies | A146665A | lx | 98% |
| Essential 8 Supplement | Life Technologies | A146665A | 50x | 2% |

DI DA Induction Medium (Day 1)

| DMEM/F12 | Life Technologies | 11330-032 | lx | | 98% | |
|---|---|---|---|---|---|---|
| B-27 Supplement (+VitA) | Life Technologies | 17504-044 | 2x | | 2% | |
| LDN-193189 | Stemgent | 04-0074 | 10 | mM | 200 | nM |
| Purmorphamine | Cayman | 10009634 | 10 | mM | 2 | μM |
| C25II SHH | R&D Systems | 464-SH | 100 | μg/mL | 100 | ng/mL |

D 2 DA Induction Medium (Day 2)

| DMEM/F12 | Life Technologies | 11330-032 | lx | | 98% | |
|---|---|---|---|---|---|---|
| B-27 Supplement (+VitA) | Life Technologies | 17504-044 | 2x | | 2% | |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |

D 3-4 DA Induction Medium (Days 3 and 4)

| DMEM/F12 | Life Technologies | 11330-032 | lx | | 98% | |
|---|---|---|---|---|---|---|
| B-27 Supplement (−VitA) | Life Technologies | 12587-010 | 2x | | 2% | |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |
| PD0325901 | Stemgent | 04-0006 | 10 | mM | 1.0 | μM |

DA Quench Medium 1 (Days 5 and 17)

| DMEM/F12 | Life Technologies | 11330-032 | lx | 98% | |
|---|---|---|---|---|---|
| B-27 Supplement (−VitA) | Life Technologies | 12587-010 | 2x | 2% | |
| Blebbistatin | Sigma | B0560 | 10,000x | 2.5 | μM |

DS DA Aggregate Formation Medium (Day 5)

| DMEM/F12 | Life Technologies | 11330-032 | lx | | 98% | |
|---|---|---|---|---|---|---|
| B-27 Supplement (−VitA) | Life Technologies | 12587-010 | 2x | | 2% | |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |
| PD0325901 | Stemgent | 04-0006 | 10 | mM | 1.0 | μM |

| | | | | | |
|---|---|---|---|---|---|
| Blebbistatin | Sigma | B0560 | 2,500x | 10 | μM |

D 6-7 DA Induction Medium (Day 6-7)

| DMEM/F12 | Life Technologies | 11330-032 | lx | | 98% | |
|---|---|---|---|---|---|---|
| B-27 Supplement (−VitA) | Life Technologies | 12587-010 | 2x | | 2% | |
| LDN-193189 | Stemgent | 04-0074 | 10 | mM | 200 | nM |
| Purmorphamine | Cayman | 10009634 | 10 | mM | 2 | μM |
| C2511 SHH | R&D Systems | 464-SH | 100 | μg/mL | 100 | ng/mL |
| CHIR99021 | Stemgent | 04-0004 | 20 | mM | 1.75 | μM |

D 8-10 DA Aggregate Maintenance Medium (Days 8-10)

| DMEM/F12 | Life Technologies | 11330-032 | lx | | 98% | |
|---|---|---|---|---|---|---|
| B-27 Supplement (−VitA) | Life Technologies | 12587-010 | 2x | | 2% | |
| LDN-193189 | Stemgent | 04-0074 | 10 | mM | 200 | nM |
| CHIR99021 | Stemgent | 04-0004 | 20 | mM | 1.75 | μM |

D 11-16 DA Aggregate Maintenance Medium (Days 11-16)

| DMEM/F12 | Life Technologies | 11330-032 | lx | | 98% | |
|---|---|---|---|---|---|---|
| B-27 Supplement (−VitA) | Life Technologies | 12587-010 | 2x | | 2% | |
| LDN-193189 | Stemgent | 04-0074 | 10 | mM | 200 | nM |
| CHIR99021 | Stemgent | 04-0004 | 20 | mM | 3.0 | μM |
| FGF8 | R&D Systems | 423-F8 | 50 | μg/mL | 100 | ng/mL |

D 17 DA Aggregate Plating Medium (Days 17)

| DMEM/F12 | Life Technologies | 11330-032 | lx | | 98% | |
|---|---|---|---|---|---|---|
| B-27 Supplement (−VitA) | Life Technologies | 12587-010 | 2x | | 2% | |
| LDN-193189 | Stemgent | 04-0074 | 10 | mM | 200 | nM |
| CHIR99021 | Stemgent | 04-0004 | 20 | mM | 3.0 | μM |
| FGF8 | R&D Systems | 423-F8 | 50 | μg/mL | 100 | ng/mL |

| | | | | | |
|---|---|---|---|---|---|
| Blebbistatin | Sigma | B0560 | 10,000x | 2.5 | μM |

DA Maturation Medium (Days 18+)

| Neurobasal | Life Technologies | 21103-049 | lx | | 98% | |
|---|---|---|---|---|---|---|
| Glutamax | Life Technologies | 35050-061 | 200 | mM | 2 | mM |
| B-27 Supplement (−VitA) | Life Technologies | 12587-010 | 2x | | 2% | |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/mL |
| dbcAMP | Sigma | D0627 | 100 | mM | 500 | μM |
| DAPT | Sigma | D5942 | 20 | mM | 5 | μM |
| Neurobasal | Life Technologies | 21103-049 | lx | | 98% | |
| Glutamax | Life Technologies | 35050-061 | 200 | mM | 2 | mM |
| B-27 Supplement (−VitA) | Life Technologies | 12587-010 | 2x | | 2% | |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/mL |
| dbcAMP | Sigma | D0627 | 100 | mM | 500 | μM |
| DAPT | Sigma | D5942 | 20 | mM | 5 | μM |
| Mitomycin C | Sigma | M4287 | 1 | mg/mL | 100 | ng/mL |

DA Quench Medium 2 (Days 18+)

| Neurobasal | Life Technologies | 21103-049 | lx | | 98% | |
|---|---|---|---|---|---|---|
| Glutamax | Life Technologies | 35050-061 | 200 | mM | 2 | mM |

| B-27 Supplement (−VitA) | Life Technologies | 12587-010 | 2x | 2% | |
|---|---|---|---|---|---|
| Blebbistatin | Sigma | B0560 | 10,000x | 2.5 | μM |

DA Maturation Plating Medium (Days 18+)

| Neurobasal | Life Technologies | 21103-049 | lx | | 98% | |
|---|---|---|---|---|---|---|
| Glutamax | Life Technologies | 35050-061 | 200 | mM | 2 | mM |
| B-27 Supplement (−VitA) | Life Technologies | 12587-010 | 2x | | 2% | |
| Ascorbic Acid | Sigma | A4403 | 200 | mM | 200 | μM |
| Rec Hu BDNF | R&D Systems | 248-BD | 20 | μg/mL | 20 | ng/mL |
| Rec Hu GDNF | R&D Systems | 212-GD | 20 | μg/mL | 20 | ng/mL |
| Rec Hu TGFB3 | R&D Systems | 243-B3 | 10 | μg/mL | 1 | ng/mL |
| dbcAMP | Sigma | D0627 | 100 | mM | 500 | μM |
| DAPT | Sigma | D5942 | 20 | mM | 5 | μM |

| | | | | | |
|---|---|---|---|---|---|
| Blebbistatin | Sigma | B0560 | 10,000x | 2.5 | μM |

In some embodiments, the DA neurons produced from differentiating iPSCs comprise three distinct cell populations: A9 dopamine neurons, astrocytes and vascular leptomeningeal cells (VLMC). In some embodiments, the A9 dopamine neurons are miR-155-3p-biased. In some embodiments, the A9 dopamine neurons are miR-155-5p-biased. In some embodiments, the A9 dopamine neurons are miR-155-5p-un-biased. In some embodiments, the astrocytes are miR-155-3p-biased. In some embodiments, the astrocytes are miR-155-5p-biased. In some embodiments, the astrocytes are miR-155-5p-un-biased. In some embodiments, the VLMCs are miR-155-3p-biased. In some embodiments, the VLMCs are miR-155-5p-biased. In some embodiments, the VLMCs are miR-155-5p-un-biased.

In some embodiments, the A9 dopamine neurons are miR-155-3p-biased and are isolated from the astrocytes and vascular leptomeningeal cells. In some embodiments, the A9 dopamine neurons are miR-155-5p-biased and are isolated from the astrocytes and vascular leptomeningeal cells. In some embodiments, the A9 dopamine neurons are miR-155-5p-unbiased and are isolated from the astrocytes and vascular leptomeningeal cells. In some embodiments, the astrocytes are miR-155-3p-biased and are isolated from the A9 dopamine neurons and vascular leptomeningeal cells. In some embodiments, the astrocytes are miR-155-5p-biased and are isolated from the A9 dopamine neurons and vascular leptomeningeal cells. In some embodiments, the astrocytes are miR-155-5p-unbiased and are isolated from the A9 dopamine neurons and vascular leptomeningeal cells. In some embodiments, the VLMCs are miR-155-3p-biased and are isolated from the A9 dopamine neurons and astrocytes. In some embodiments, the VLMCs are miR-155-5p-biased and are isolated from the A9 dopamine neurons and astrocytes. In some embodiments, the VLMCs are miR-155-5p-unbiased and are isolated from the A9 dopamine neurons and astrocytes.

In some embodiments, the DA neurons produced from differentiating iPSCs are further differentiated by adding an antilipemic agent or CSF-1R antagonist. The DA neurons produced from differentiating iPSCs are further differentiated by adding an antilipemic agent. The DA neurons produced from differentiating iPSCs are further differentiated by adding fenofibrate. The DA neurons produced from differentiating iPSCs are further differentiated by adding fenofibrate. The DA neurons produced from differentiating iPSCs are further differentiated by adding a CSF-1R antagonist. The DA neurons produced from differentiating iPSCs are further differentiated by adding pexidartinib.

In some embodiments, The DA neurons produced from differentiating iPSCs are further differentiated by adding an antilipemic agent and a CSF-TR antagonist. The DA neurons produced from differentiating iPSCs are further differentiated by adding fenofibrate and a CSF-TR antagonist. The DA neurons produced from differentiating iPSCs are further differentiated by adding fenofibrate and a CSF-1R antagonist. The DA neurons produced from differentiating iPSCs are further differentiated by adding fenofibrate and pexidartinib. The DA neurons produced from differentiating iPSCs are further differentiated by adding fenofibrate and pexidartinib.

In some embodiments, the DA neurons produced from differentiating iPSCs are incubated with an antilipemic agent or CSF-TR antagonist. The DA neurons produced from differentiating iPSCs are incubated with an antilipemic agent. The DA neurons produced from differentiating iPSCs are incubated with fenofibrate. The DA neurons produced from differentiating iPSCs are incubated with fenofibrate. The DA neurons produced from differentiating iPSCs are incubated with a CSF-TR antagonist. The DA neurons produced from differentiating iPSCs are incubated with pexidartinib.

In some embodiments, The DA neurons produced from differentiating iPSCs are incubated with an antilipemic agent and a CSF-TR antagonist. The DA neurons produced from differentiating iPSCs are incubated with fenofibrate and a CSF-1R antagonist. The DA neurons produced from differentiating iPSCs are incubated with fenofibrate and a CSF-1R antagonist. The DA neurons produced from differentiating iPSCs are incubated with fenofibrate and pexidartinib. The DA neurons produced from differentiating iPSCs are incubated with fenofibrate and pexidartinib.

Further enrichment of a particular cell type may be affected using cell sorting techniques such as FACS and magnetic sorting.

In addition, cell differentiation can be also followed by specific reporters that are tagged with GFP or RFP and exhibit increased fluorescence upon differentiation.

### Isolation of Particular Subpopulations of Cells

Isolation of particular subpopulations of cells may be affected using techniques known in the art including fluorescent activated cell sorting and/or magnetic separation of cells.

### Method of Treatment

Disclosed is a method to treat Parkinson's Disease or other Parkinsonian disorders in a subject in need thereof comprising administering to a subject in need thereof a therapeutic amount of DA neurons and an antilipemic agent or CSF-1R antagonist. Disclosed is a method to treat Parkinson's Disease in a subject in need thereof comprising administering to a subject in need thereof a therapeutic amount of DA neurons and an antilipemic agent. Disclosed is a method to treat Parkinson's Disease in a subject in need thereof comprising administering to a subject in need thereof a therapeutic amount of DA neurons and fenofibrate. Disclosed is a method to treat Parkinson's Disease in a subject in need thereof comprising administering to a subject in need thereof a therapeutic amount of DA neurons and fenofibrate. Disclosed is a method to treat Parkinson's Disease in a subject in need thereof comprising administering to a subject in need thereof a therapeutic amount of DA neurons and a CSF-1R antagonist. Disclosed is a method to treat Parkinson's Disease in a subject in need thereof comprising administering to a subject in need thereof a therapeutic amount of DA neurons and pexidartinib.

Disclosed is a method to treat Parkinson's Disease in a subject in need thereof comprising administering to a subject in need thereof a therapeutic amount of DA neurons and an antilipemic agent and a CSF-1R antagonist. Disclosed is a method to treat Parkinson's Disease in a subject in need thereof comprising administering to a subject in need thereof a therapeutic amount of DA neurons and fenofibrate and a CSF-1R antagonist. Disclosed is a method to treat Parkinson's Disease in a subject in need thereof comprising administering to a subject in need thereof a therapeutic amount of DA neurons and fenofibrate and a CSF-1R antagonist. Disclosed is a method to treat Parkinson's Disease in a subject in need thereof comprising administering to a subject in need thereof a therapeutic amount of DA neurons and fenofibrate and pexidartinib. Disclosed is a method to treat Parkinson's Disease in a subject in need thereof comprising administering to a subject in need thereof a therapeutic amount of DA neurons and fenofibrate and pexidartinib.

Disclosed is a method to rescue or increase survival of dopamine neurons in a subject in need thereof comprising administering to a subject in need thereof a therapeutic amount of DA neurons and an antilipemic agent or CSF-TR antagonist. Disclosed is a method to rescue or increase survival of dopamine neurons in a subject in need thereof comprising administering to a subject in need thereof a therapeutic amount of DA neurons and an antilipemic agent. Disclosed is a method to rescue or increase survival of dopamine neurons in a subject in need thereof comprising administering to a subject in need thereof a therapeutic amount of DA neurons and fenofibrate. Disclosed is a method to rescue or increase survival of dopamine neurons in a subject in need thereof comprising administering to a subject in need thereof a therapeutic amount of DA neurons and fenofibrate. Disclosed is a method to rescue or increase survival of dopamine neurons in a subject in need thereof comprising administering to a subject in need thereof a therapeutic amount of DA neurons and a CSF-1R antagonist. Disclosed is a method to rescue or increase survival of dopamine neurons in a subject in need thereof comprising administering to a subject in need thereof a therapeutic amount of DA neurons and pexidartinib.

Disclosed is a method to rescue or increase survival of dopamine neurons in a subject in need thereof comprising administering to a subject in need thereof a therapeutic amount of DA neurons and an antilipemic agent and a CSF-TR antagonist. Disclosed is a method to rescue or increase survival of dopamine neurons in a subject in need thereof comprising administering to a subject in need thereof a therapeutic amount of DA neurons and fenofibrate and a CSF-TR antagonist. Disclosed is a method to rescue or increase survival of dopamine neurons in a subject in need thereof comprising administering to a subject in need thereof a therapeutic amount of DA neurons and fenofibrate and a CSF-1R antagonist. Disclosed is a method to rescue or increase survival of dopamine neurons in a subject in need thereof comprising administering to a subject in need thereof a therapeutic amount of DA neurons and fenofibrate and pexidartinib. Disclosed is a method to rescue or increase survival of dopamine neurons in a subject in need thereof comprising administering to a subject in need thereof a therapeutic amount of DA neurons and fenofibrate and pexidartinib.

Modified Treatment with Modified DA Neurons
Therapeutic Amount of miR-155-3p Biased DA Neurons and an Antilipemic Agent and/or CSF-1R Antagonist.

Disclosed is a method to treat Parkinson's Disease in a subject in need thereof comprising administering to a subject in need thereof a therapeutic amount of miR-155-3p biased DA neurons and an antilipemic agent and/or CSF-1R antagonist. Disclosed is a method to treat Parkinson's Disease in a subject in need thereof comprising administering to a subject in need thereof a therapeutic amount of miR-155-3p biased DA neurons and an antilipemic agent. Disclosed is a method to treat Parkinson's Disease in a subject in need thereof comprising administering to a subject in need thereof a therapeutic amount of miR-155-3p biased DA neurons and fenofibrate. Disclosed is a method to treat Parkinson's Disease in a subject in need thereof comprising administering to a subject in need thereof a therapeutic amount of miR-155-3p biased DA neurons and fenofibrate. Disclosed is a method to treat Parkinson's Disease in a subject in need thereof comprising administering to a subject in need thereof a therapeutic amount of miR-155-3p biased DA neurons and a CSF-1R antagonist. Disclosed is a method to treat Parkinson's Disease in a subject in need thereof comprising administering to a subject in need thereof a therapeutic amount of miR-155-3p biased DA neurons and pexidartinib.

Disclosed is a method to treat Parkinson's Disease in a subject in need thereof comprising administering to a subject in need thereof a therapeutic amount of miR-155-3p biased DA neurons and an antilipemic agent and a CSF-TR antagonist. Disclosed is a method to treat Parkinson's Disease in a subject in need thereof comprising administering to a subject in need thereof a therapeutic amount of miR-155-3p biased DA neurons and fenofibrate and a CSF-TR antagonist. Disclosed is a method to treat Parkinson's Disease in a subject in need thereof comprising administering to a subject in need thereof a therapeutic amount of miR-155-3p biased DA neurons and fenofibrate and a CSF-1R antagonist. Disclosed is a method to treat Parkinson's Disease in a subject in need thereof comprising administering to a subject in need thereof a therapeutic amount of miR-155-3p biased DA neurons and fenofibrate and pexidartinib. Disclosed is a method to treat Parkinson's Disease in a subject in need thereof comprising administering to a subject in need thereof a therapeutic amount of miR-155-3p biased DA neurons and fenofibrate and pexidartinib.

Therapeutic Amount of miR-155-5p Biased DA Neurons and an Antilipemic Agent or CSF-1R Antagonist.

Disclosed is a method to treat Parkinson's Disease in a subject in need thereof comprising administering to a subject in need thereof a therapeutic amount of miR-155-5p biased DA neurons and an antilipemic agent or CSF-1R antagonist. Disclosed is a method to treat Parkinson's Disease in a subject in need thereof comprising administering to a subject in need thereof a therapeutic amount of miR-155-5p biased DA neurons and an antilipemic agent. Disclosed is a method to treat Parkinson's Disease in a subject in need thereof comprising administering to a subject in need thereof a therapeutic amount of miR-155-5p biased DA neurons and fenofibrate. Disclosed is a method to treat Parkinson's Disease in a subject in need thereof comprising administering to a subject in need thereof a therapeutic amount of miR-155-5p biased DA neurons and fenofibrate. Disclosed is a method to treat Parkinson's Disease in a subject in need thereof comprising administering to a subject in need thereof a therapeutic amount of miR-155-3p biased DA neurons and a CSF-TR antagonist. Disclosed is a method to treat Parkinson's Disease in a subject in need thereof comprising administering to a subject in need thereof a therapeutic amount of miR-155-5p biased DA neurons and pexidartinib.

Disclosed is a method to treat Parkinson's Disease in a subject in need thereof comprising administering to a subject in need thereof a therapeutic amount of miR-155-5p biased DA neurons and an antilipemic agent and a CSF-1R antagonist. Disclosed is a method to treat Parkinson's Disease in a subject in need thereof comprising administering to a subject in need thereof a therapeutic amount of miR-155-5p biased DA neurons and fenofibrate and a CSF-TR antagonist. Disclosed is a method to treat Parkinson's Disease in a subject in need thereof comprising administering to a subject in need thereof a therapeutic amount of miR-155-5p biased DA neurons and fenofibrate and a CSF-1R antagonist. Disclosed is a method to treat Parkinson's Disease in a subject in need thereof comprising administering to a subject in need thereof a therapeutic amount of miR-155-5p biased DA neurons and fenofibrate and pexidartinib. Disclosed is a method to treat Parkinson's Disease in a subject in need thereof comprising administering to a subject in need thereof a therapeutic amount of mi-155-5p biased DA neurons and fenofibrate and pexidartinib.

Therapeutic Amount of DA Neurons with a Modified MIR-155 and an Antilipemic Agent or CSF-1R Antagonist.

Disclosed is a method to rescue or increase survival of dopamine neurons in a subject in need thereof comprising administering to a subject in need thereof a therapeutic amount of DA neurons with a modified MIR-155 and an antilipemic agent or CSF-TR antagonist. Disclosed is a method to rescue or increase survival of dopamine neurons in a subject in need thereof comprising administering to a subject in need thereof a therapeutic amount of DA neurons with a modified MIR-155 and an antilipemic agent. Disclosed is a method to rescue or increase survival of dopamine neurons in a subject in need thereof comprising administering to a subject in need thereof a therapeutic amount of DA neurons with a modified MIR-155 and fenofibrate. Disclosed is a method to rescue or increase survival of dopamine neurons in a subject in need thereof comprising administering to a subject in need thereof a therapeutic amount of DA neurons with a modified MIR-155 and fenofibrate. Disclosed is a method to rescue or increase survival of dopamine neurons in a subject in need thereof comprising administering to a subject in need thereof a therapeutic amount of DA neurons with a modified MIR-155 and a CSF-TR antagonist. Disclosed is a method to rescue or increase survival of dopamine neurons in a subject in need thereof comprising administering to a subject in need thereof a therapeutic amount of DA neurons with a modified MIR-155 and pexidartinib.

Disclosed is a method to rescue or increase survival of dopamine neurons in a subject in need thereof comprising administering to a subject in need thereof a therapeutic amount of DA neurons with a modified MIR-155 and an antilipemic agent and a CSF-TR antagonist. Disclosed is a method to rescue or increase survival of dopamine neurons in a subject in need thereof comprising administering to a subject in need thereof a therapeutic amount of DA neurons with a modified MIR-155 and fenofibrate and a CSF-1R antagonist. Disclosed is a method to rescue or increase survival of dopamine neurons in a subject in need thereof comprising administering to a subject in need thereof a therapeutic amount of DA neurons with a modified MIR-155 and fenofibrate and a CSF-TR antagonist. Disclosed is a method to rescue or increase survival of dopamine neurons in a subject in need thereof comprising administering to a subject in need thereof a therapeutic amount of DA neurons with a modified MIR-155 and fenofibrate and pexidartinib. Disclosed is a method to rescue or increase survival of dopamine neurons in a subject in need thereof comprising administering to a subject in need thereof a therapeutic amount of DA neurons with a modified MIR-155 and fenofibrate and pexidartinib.

The compositions are useful for the preparation of a medicament and/or to perform methods for one or more of a) inhibiting the progression of, b) preventing or c) treating, a disease, e.g., a neurologic disease or an associated disorder.

In one aspect, the compositions are useful for the preparation of a medicament and/or to perform methods for one or more of: a) inhibiting the progression of, b) preventing or c) treating, Parkinson's disease or an associated disorder in a subject in need thereof. The methods comprise, or alternatively consist essentially of, or yet further consist of, administering to the subject an effective amount of the pharmaceutical composition described above including miR-155-3p biased DA neural cells or miR-155-5p biased DA neural cells.

As noted, the cells are administered in an effective amount. For example, an effective amount comprises from about 1 to about 1,000 mg/kg, or alternatively from about 1 to about 500 mg/kg, or alternatively from about 5 to about 500 mg/kg, or alternatively from about 10 to about 100 mg/kg, or alternatively from about 5 mg/kg to about 100 mg/kg, or alternatively from about 10 mg/kg to about 80 mg/kg, or alternatively from about 10 mg/kg to about 50 mg/kg, or alternatively from about 15 mg/kg to about 50 mg/kg, or alternatively more than 5 mg/kg, or alternatively more than about 10 mg/kg, or alternatively more than about 15 mg/kg, or alternatively more than about 20 mg/kg, or alternatively more than 25 mg/kg, or alternatively more than 30 mg/kg, each as measured per kg of body weight of the subject. The effective amount is in one aspect, per dose, or as a daily dose, or alternatively the total over the course of treatment.

### Transplantation

The disclosed administered cells can be administered to the treated individual using a variety of transplantation approaches, the nature of which depends on the site of implantation.

The disclosed administered cells can be injected systemically into the circulation, administered intrathecally or grafted into the central nervous system, the spinal cord or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the brain parenchyma at the time of transplantation.

Intraparenchymal transplantation can be performed using two approaches: (i) injection of cells into the host brain parenchyma or (ii) preparing a cavity by surgical means to expose the host brain parenchyma and then depositing the graft into the cavity.

Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain may be made by drilling a hole and piercing the dura to permit the needle of a micro syringe to be inserted. The micro syringe is preferably mounted in a stereotaxic frame and three-dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The disclosed administered cells may also be transplanted to a healthy region of the tissue. The administered cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the disclosed cell suspension is drawn up into the syringe and administered to anesthetized transplantation recipients. Multiple injections may be made using this procedure. For transplanting, the disclosed cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions.

Multiple grafts may consist of a mixture of cell types, and/or a mixture of transgenes inserted into the cells. Preferably from approximately 104 to approximately 109 cells are introduced per graft. Cells can be administered concomitantly to different locations such as combined administration intrathecally and intravenously to maximize the chance of targeting into affected areas.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20, 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated. Demyelinated MS lesions are distributed across multiple locations throughout the CNS, such that effective treatment of MS may rely more on the migratory ability of the cells to the appropriate target sites.

Intranasal administration of the disclosed administered cells is also contemplated.

The use of pluripotent cells or differentiated pluripotent cells may be also indicated for treatment of traumatic lesions of the nervous system including spinal cord injury and also for treatment of stroke caused by bleeding or thrombosis or embolism because of the need to induce neurogenesis and provide survival factors to minimize insult to damaged neurons.

Since non-autologous cells may induce an immune reaction when administered to the body several approaches have been developed to reduce the likelihood of rejection of non-autologous cells. Furthermore, since diseases such as multiple sclerosis are inflammatory based diseases, the problem of immune reaction is exacerbated. These include either administration of cells to privileged sites, or alternatively, suppressing the recipient's immune system, providing anti-inflammatory treatment which may be indicated to control autoimmune disorders to start with and/or encapsulating the non-autologous/semi-autologous cells in immunoisolating, semipermeable membranes before transplantation.

As mentioned herein above, the present inventor also proposes use of cord and placenta-derived pluripotent cells that express very low levels of MHCII molecules and therefore limit immune response.

The following experiments may be performed to confirm the potential use of newborn's pluripotent cells isolated from the cord I placenta for treatment of neurological disorders:

1. Differentiated pluripotent cells (to various neural cells or neural progenitor cells) may serve as stimulators in one-way mixed lymphocyte culture with allogeneic T-cells and proliferative responses in comparison with T cells responding against allogeneic lymphocytes isolated from the same donor may be evaluated by 3H Thymidine uptake to document hyporesponsiveness.

2. Differentiated pluripotent cells may be added/co-cultured to one-way mixed lymphocyte cultures and to cell cultures with T cell mitogens (phytohemmaglutinin and concanavalin A) to confirm the immunosuppressive effects on proliferative responses mediated by T cells.

3. Cord and placenta cells cultured from Brown Norway rats (unmodified and differentiated), may be enriched for pluripotent cells and these cells may be infused into Lewis rats with induced experimental autoimmune encephalomyelitis (EAE). Alternatively, cord and placenta cells cultured from BALB/c mice, (BALB/cxC57BL/6)F1 or xenogeneic cells from Brown Norway rats (unmodified and differentiated), may be enriched for pluripotent cells and these cells may be infused into C57BL/6 or SJL/j recipients with induced experimental autoimmune encephalomyelitis (EAE). The clinical effects against paralysis may be investigated to evaluate the therapeutic effects of xenogeneic, fully MHC mismatched or haploidentically mismatched pluripotent cells. Such experiments may provide the basis for treatment of patients with a genetic disorder or genetically proned disorder with family member's haploidentical pluripotent cells.

4. Pluripotent cells cultured from cord and placenta may be transfused with pre-miR labeled with GFP or RFP, which will allow the inventors to follow the migration and persistence of these cells in the brain of C57BL/6 recipients with induced EAE. The clinical effects of labeled MHC mismatched differentiated pluripotent cells may be evaluated by monitoring signs of disease, paralysis and histopathology. The migration and localization of such cells may be also monitored by using fluorescent cells from genetically transduced GFP “green” or Red2 “red” donors.

As mentioned, the present disclosure also contemplates encapsulation techniques to minimize an immune response.

### Encapsulation Techniques

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha phenoxycinnamylidene-acetylated poly(allylamine). Biotechnol Bioeng. 2000, 70: 479-83. Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J. Microencapsul. 2000, 17: 245-51.

For example, microcapsules are prepared by complexing modified collagen with a per-polymer shell of 2-hydroxyethyl methylacrylate (HEMA), methacrylic acid (MAA) and methyl methacrylate (MMA), resulting in a capsule thickness of 2-5 um. Such microcapsules can be further encapsulated with additional 2-5 um per-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Technol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 um (Canaple L. et al., Improving cell encapsulation through size control. J Biomater Sci Polym Ed. 2002; 13:783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

### Immunosuppressive Agents

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE™), etanercept, TNF alpha blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium, salicylate, salsalate, sodium salicylate, diclofenac, etodolac, fenoprofen, flurbiprofen, indomethacin. ketoprofen, ketorolac, meclofenamate, naproxen, nabumetone, phenylbutazone, piroxicam, sulindac, tolmetin, acetaminophen, ibuprofen, Cox-2 inhibitors and tramadol.

In any of the methods described herein, the cells can be administered either per se or, preferably as a part of a pharmaceutical composition that further comprises a pharmaceutically acceptable carrier.

Techniques for formulation and administration of drugs may be found in “Remington's Pharmaceutical Sciences,” Mack Publishing Co., Easton, Pa., latest edition, which is incorporated herein by reference.

### Administration

Suitable routes of administration include direct administration into the circulation (intravenously or intra-arterial), into the spinal fluid or into the tissue or organ of interest. Thus, for example the cells may be administered directly into the brain.

For any preparation used in the methods disclosed herein, the therapeutically effective amount or dose can be estimated initially from in vitro and cell culture assays. Preferably, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. For example, animal models of demyelinating diseases include shiverer (shi/shi, MBP deleted) mouse, MD rats (PLP deficiency), Jimpy mouse (PLP mutation), dog shaking pup (PLP mutation), twitcher mouse (galactosylceramidase defect, as in human Krabbe disease), trembler mouse (PMP-22 deficiency). Virus induced demyelination model comprise use if Theiler's virus and mouse hepatitis virus. Autoimmune EAE is a possible model for multiple sclerosis.

The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1). For example, a multiple sclerosis patient can be monitored symptomatically for improved motor functions indicating positive response to treatment.

For injection, the active ingredients of the pharmaceutical composition may be formulated in aqueous solutions, preferably in physiologically compatible buffers such as Hank's solution, Ringer's solution, or physiological salt buffer.

Dosage amount and interval may be adjusted individually to levels of the active ingredient which are sufficient to effectively treat the brain disease/disorder. Dosages necessary to achieve the desired effect will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition. For example, a treated multiple sclerosis patient will be administered with an amount of cells which is sufficient to alleviate the symptoms of the disease, based on the monitoring indications.

The administered cells may be co-administered with therapeutic agents useful in treating neurodegenerative disorders, such as gangliosides; antibiotics, neurotransmitters, neurohormones, toxins, neurite promoting molecules; and antimetabolites and precursors of neurotransmitter molecules such as L-DOPA.

### Negative Limitations

The Composition or Combination Therapy does not Include any Compositions as Described in US Patent Publication Number 20170081326A1

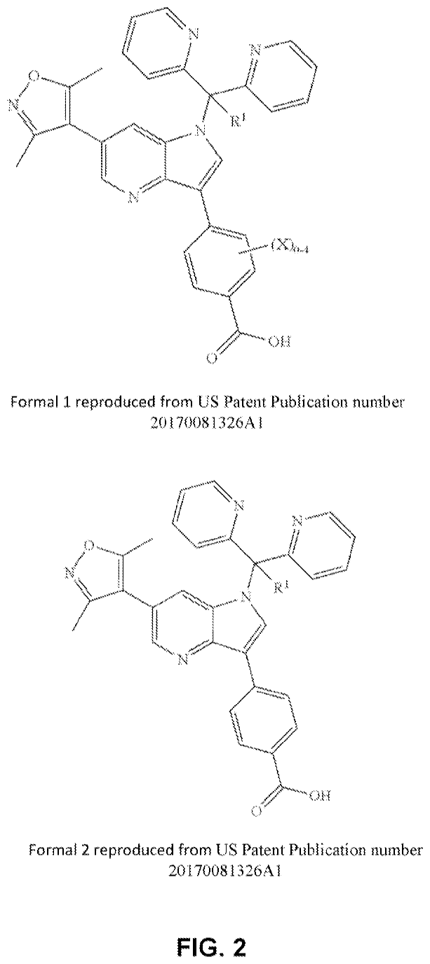

It is contemplated that for each of the compounds, and combination therapies described above the composition or combination therapy does not include Formulae (I) or (II) (reproduced in FIG. 2) or a pharmaceutically acceptable salt, a solvate, a tautomer, an isomer, or a deuterated analog of Formulae (I) or (II) as described in US Patent Publication number 20170081326A1 wherein for formula I, R1 is cyano, halo, or (C1-C3) alkyl optionally substituted with 1 to 3 substituents independently selected from the group consisting of halo, methyl, ethyl, methoxy and ethoxy; and X, when present, is halo and wherein for formula II, R¹ is cyano, halo, or (C₁-C₃)alkyl optionally substituted with 1 to 3 substituents independently selected from the group consisting of halo, methyl, ethyl, methoxy and ethoxy. Specifically, US Patent Publication number 20170081326A1 describes formula I and II as novel compounds because the compounds in the disclosure have a di(pyridin-2-yl)methylene moiety that requires a R¹ substituent as defined in the disclosure resulting in desirable PK properties. It is not obvious to remove/exclude formula I and/or formula II from the compositions and combination therapies described therein because a skilled artisan would consider PK properties desirable.

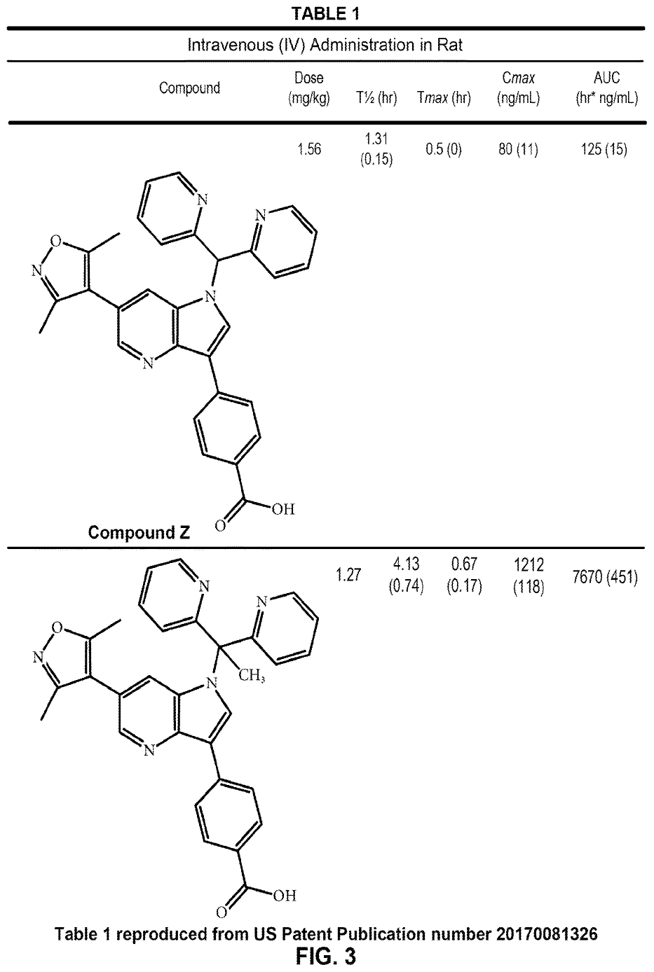

It is contemplated that for each of the compounds, and combination therapies described above the composition or combination therapy does not include any of the compounds in Table I (reproduced in FIG. 3) of US Patent Publication number 20170081326A1.

It is contemplated that for each of the compounds, and combination therapies described above the composition or combination therapy does not include any compositions as described in US Patent Publication number 20170081326A1.

The Composition or Combination Therapy does not Include any Compositions as Described in WO2014145051A1

It is contemplated that for each of the compounds, and combination therapies described above the composition or combination therapy does not include any heterocyclic compounds of formula (I), as described in WO2014145051A1. It is contemplated that for each of the compounds, and combination therapies described above the composition or combination therapy does not include any heterocyclic compounds of formula (I), (Γ) (II), (III), (IV), (V), (Va) or (Vb), or any of the formulas and subformulas as described in WO2014145051A1, or a compound as recited in any of the claims and described in WO2014145051A1, or a pharmaceutically acceptable salt, solvate, tautomer or isomers thereof, or a pharmaceutical composition as described in WO2014145051A1.

It is contemplated that for each of the compounds, and combination therapies described above the composition or combination therapy does not include any pharmaceutical composition as described in WO2014145051A1 and US Patent Publication number 20170081326A1.

The Composition or Combination Therapy does not Include any Compositions as Described in U.S. Pat. No. 10,717,735

It is contemplated that for each of the compounds, and combination therapies described above the composition or combination therapy does not include any pharmaceutical composition as described in U.S. Pat. No. 10,717,735. It is contemplated that for each of the compounds, and combination therapies described above the composition or combination therapy does not include the free acid amorphous form of Compound I or a pharmaceutically acceptable salt, a solvate, a tautomer, an isomer, or a deuterated analog of Compound I as described in U.S. Pat. No. 10,717,735

It is contemplated that for each of the compounds, and combination therapies described above the composition or combination therapy does not include any pharmaceutical composition as described in WO2014145051A1, US Patent Publication number 20170081326A1 and U.S. Pat. No. 10,717,735.

### Kits

A kit is provided which facilitates administering DA neuronal cells.

Kits may further comprise suitable packaging and/or instructions for use of the compositions and/or administration of the administered cells. Kits may also comprise a means for the delivery of the at least one composition, and syringe for injection.

Additionally, the kits can contain the composition and reagents to prepare a modified or un-modified DA neural cell composition for administration. The kit may contain a device for administration or for dispensing the compositions, including, but not limited to, syringe, pipette, or transdermal patch.

The kits may include other therapeutic compounds for use in conjunction with the compounds described herein and as such, the methods as disclosed herein can contain other appropriate therapeutic compounds or agents. These compounds can be provided in a separate form or mixed with the modified or un-modified DA neural cell compositions of the present disclosure. These compounds can be provided in a separate form or mixed with the un-modified DA neural cell compositions of the present disclosure. The kits will include appropriate instructions for preparation and administration of the modified or un-modified DA neural cell composition, side effects of the compositions, and any other relevant information. The instructions can be in any suitable format, including, but not limited to, printed matter, videotape, computer readable disk, or optical disc.

Kits may also be provided that contain sufficient dosages of the compounds or composition to provide effective treatment for an individual for an extended period, such as a week, 2 weeks, 3, weeks, 4 weeks, 6 weeks, or 8 weeks or more.

A kit comprising compounds necessary to modify pre-miR-155-5p or pre-miR-155-3p DA neurons and an antilipemic agent and/or CSF-TR antagonist. A kit comprising compounds necessary to differentiate pluripotent cells to miR-155-5p or miR-155-3p DA neurons and an antilipemic agent and/or CSF-TR antagonist. A kit comprising compounds necessary to differentiate pluripotent cells to miR-155-5p biased or miR-155-3p biased DA A9 dopamine neurons, astrocytes and vascular leptomeningeal cells (VLMC) and an antilipemic agent and/or CSF-TR antagonist.

A kit also is provided for one or more of a) inhibiting the progression of, b) preventing or c) treating, Parkinson's disease or an associated disorder in a subject in need thereof, comprising an effective amount of the isolated or purified cell populations and/or the pharmaceutical composition as described above and/or reagents and/or instructions for use.

The following examples are intended to illustrate, and not limit, the disclosed herein. For example, while the examples are noted to be for the isolation, purification and use of exosome compositions for the treatment of a fibrotic or liver disease or an associated disorder, the methods and compositions can be modified for the treatment of other neurologic diseases as noted herein.

### PROPHETIC EXAMPLES

### Example 1

Survival of Neural Cells is Supported by an Antilipemic Agent and/or a CSF-1R Antagonist

DA neuronal cells exposed to an antilipemic agent and/or a CSF-TR antagonist. It is expected that an antilipemic agent and/or a CSF-TR antagonist promotes neuron survival and inhibits the appearance and differentiation of monocytes derivatives (macrophages/microglia) in vitro and in vivo, including after systemic administration.

### Example 2

An Antilipemic Agent and/or a CSF-1R Antagonist is Expected to Protect Neural Cells Subjected to Stress In Vitro

Various concentrations of an antilipemic agent (fenofibrate) and/or a CSF-TR antagonist (pexidartinib) will be tested in a stress test consisting of medium change followed by serum deprivation. It is expected that fenofibrate and/or pexidartinib will rescue DA neural cells when used at concentrations of 0.1 and 0.01 μM (10-100 picomolar). DA neural cell survival will be measured by either counting surviving attached DA neural cells or by colorimetric determination after applying an electrocoupling reagent that responds to chemical reactions in normal cellular respiration. The number of neurons protected in the cultures is estimated to be between 3 and 10 fold, depending on the length of serum deprivation and the starting concentration of cells.

### Example 3

Fenofibrate and Pexidartinib Protect Cerebral Cortex Neurons after Cortical Stab Wounds in Rats

Stab wounds will be administered to the rostral cortical area 3 of the exposed cerebral cortex (with dura intact) of rats, using a dissecting knife (blade=1×2 mm). The wound typically produces a significant local inflammatory response, disruption of the functional layers of the cortex, and marked atrophy and degeneration of neurons. The principal immune cells involved in the inflammatory response are macrophages and microglia. Subcutaneous injection of fenofibrate and pexidartinib (0.4 mg/Kg bilaterally in the skin of the shoulder) as well as DA neural cells, 20 min after placing the wound, is expected to result in a significant anatomical sparing of the perilesion parenchyma, as well as a more restricted inflammatory response.

### Example 4

### Activation of Microglia Cells is Inhibited by Fenofibrate and Pexidartinib and DA Neural Cells

Microglia cells will be purified from neonatal rats according to established procedures and allowed to develop for an additional 72-96 hrs in vitro, after 10 which the cells are found to be 90-98% ED-1+. ED-1 is a marker specific for rat microglia. Contaminating cells are expected to be GFAF+ (suggesting they are astrocytes) or unreactive. TNFα immunoreactivity is expected to be at moderate to low levels in these cultures. If, however, the cells are activated with 100 nM retinoic acid on days 1 and 2 in vitro and examined on day 3 or 4, the ED-1 positive microglia cells display rounded morphology with small or blunt processes suggesting that the cells are transformed into amoeboid microglia. (sometimes referred to as brain macrophages, Milligan, et al 1991a;b). TNFα immunoreactivity is more intense in these cultures. These same morphological changes have been described in several studies of microglial activation in vitro (e.g., Siao and Tsirka, 2002; Bothatschek, 2001). When the microglia are treated with 1 nM fenofibrate and pexidartinib and DA neuronal cells during the period of activation by RA (30 min after the RA treatment), the cells in the treated cultures are expected to be on average smaller with distinct processes suggesting the transformation to the activated amoeboid morphology is inhibited. Likewise, TNFα immunostaining in the cells is expected to be reduced.

### Example 5

### Fenofibrate, Pexidartinib and DA Neural Cells Protects Neural Cells and Inhibits Inflammation

Animals, Surgery, and histology: lesion studies will be conducted on 15 long evans hooded rats weighing 225-275 g. Twelve of these rats will be deeply anesthetized 10 with ketamine/xylazine and placed in a sterotaxic holder. A 4×2 mm (rostrocaudal×mediolateral) skull opening will be made on the right side starting just behind the coronal suture and centered at a mediolateral position of +2.5 mm relative to bregma. A dissecting knife will be penetrated through the dura and cortex in the center of this skull opening to a depth of 1 mm. The skull defect will be filled with bone wax, the skin sutured closed, and the animal placed on a heating pad. Twenty minutes later, 0.4 cc of solution containing 100 μg of 20 peptide (^{~}0.4 mg/kg, 6 rats) or DMEM vehicle (6 rats) will be injected under the skin of the shoulder near the midline. The rats will be perfused 4 days later and their brains processed for histology and immunohistochemistry as in previous studies. Three rats will be sacrificed without surgery or treatment. Alternate coronal sections of these brains were stained with cresyl violet acetate and immunostained with the TUJ1 antibody to neuronal specific tubulin, isotype Ill (Covance Research Products) or monocyte marker ED-1 Serotec. Secondary antibodies were FITC or Rhodamine conjugated (Jackson Immunolabs). The density of ED-1+ ameboid microglia in the perilesion parencyma wound will be calculated after experimentally blinded counting of 4 fields (dorsal and ventral margins of the wound) in 2 sections through the lesion.

In rats injected with Fenofibrate and/or Pexidartinib and DA neural cells both the disruption of the cerebral cortex and accumulation of inflammatory cells in the parenchyma is expected to be inhibited.

1. A method for promoting mitochondrial homeostasis in engrafted dopaminergic progenitors comprising administering a composition comprising an effective amount of at least one antilipemic agent to a human in need thereof.

2. The method according to claim 1, wherein said at least one antilipemic agent is fenofibrate.

3. The method according to claim 1, wherein said at least one antilipemic agent is administered in a sub-therapeutic dose.

4. The method according to claim 1, wherein said at least one antilipemic agent is administered in a therapeutic dose.

5. The method according to claim 1, wherein said effective amount is between 20 and 200 mg/day.

6. The method according to claim 1, wherein said administering is by at least one mode selected from parenteral, subcutaneous, intramuscular, intravenous, intrarticular, intracapsular, intracartilaginous, intracavitary, intraosteal, intrapelvic, intraperitoneal, intraspinal, intrasynovial, oral, buccal, sublingual, intranasal, and transdermal.

7. The method according to claim 1, further comprising administering at least one CSF-1R antagonist.

8. The method according to claim 1, further comprising administering pexidartinib.

9. The method according to claim 1, further comprising administering at least one cell population selected from the group comprising stem cells, precursor cells, progenitor cells, DA neuronal cells, neurons, brain cells, heart cells, liver cells, muscle cells or combinations thereof.

10. The method according to claim 1, wherein the composition does not comprise Formula (I) or Formula (II) or a pharmaceutically acceptable salt, a solvate, a tautomer, an isomer, or a deuterated analog of Formulae (I) or (II).

11. The method according to claim 1, wherein said at least one antilipemic agent is administered before, during or after engraftment of the dopaminergic progenitors.

12. The method according to claim 11, wherein said at least one antilipemic agent is administered at least 10 days prior to engraftment of the dopaminergic progenitors.

13. The method according to claim 1, wherein the dopaminergic progenitors are contacted with an antilipemic agent prior to engraftment.

14. The method according to claim 1, wherein the dopaminergic progenitors are genetically modified.

15. The method according to claim 1, wherein the dopaminergic progenitors are genetically modified to be miR-155-3p biased or miR-155-5p biased.

16. A method for reducing symptoms of an inflammatory condition in an animal, comprising:
administering a composition comprising an effective amount of at least one antilipemic agent to the animal, wherein said at least one antilipemic agent enhances engraftment of progenitor cells.

17. The method of claim 16, further comprising administering at least one other therapeutic agent.

18. The method of claim 16, wherein the at least one other therapeutic agent is i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from the group consisting of azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an antibody therapy agent selected from alemtuzumab, bevacizumab, cetuximab, galiximab, gemtuzumab, nivolumab, panitumumab, pembrolizumab, pertuzumab, rituximab, tositumomab, trastuzumab, and 90 Y ibritumomab tiuxetan; v) a hormone or hormone antagonist selected from the group consisting of anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI 287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38 (7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, AEE-788, AG-013736, AMG 706, AMN107, BMS-354825, BMS-599626, UCN-01 (7-hydroxystaurosporine), vemurafenib, dabrafenib, trametinib, cobimetinib selumetinib and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; and xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, a mTOR inhibitor, a PI3K inhibitor, a Cdk4 inhibitor, an Akt inhibitor, a Hsp90 inhibitor, a farnesyltransferase inhibitor or an aromatase inhibitor (anastrozole letrozole exemestane); xvi) a Mek inhibitor; xvii) a tyrosine kinase inhibitor; xviii) a c-Kit mutant inhibitor, xix) an EGFR inhibitor, or xx) an epigenetic modulator.

19. The method of claim 16, wherein the inflammatory condition is selected from the group comprising inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholecystitis, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, Type I diabetes, Type 2 diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleracierma, mycosis fungoides, acute inflammatory responses (such as acute respiratory distress syndrome and ischemia/reperfusion injury), and Graves' disease.

20. A method for reducing symptoms of Parkinson's disease or other primary and secondary Parkinsonian disorders in an animal, comprising:
administering DA neuronal cells to the animal; and
administering a composition comprising an effective amount of at least one antilipemic agent to the animal, wherein said at least one antilipemic agent enhances engraftment of the DA neuronal cells.
